SEQUENCE LISTING (64 sequences)

SEQ ID NO: 1 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 2 - DNA, 570 bp, Homo sapiens
cacctgcaccccgcccgggcatagcaccatgcctgcttgtcgcctaggcccgctagccgccgccctcctcctcagcctgctgctgttcggcttcaccctagtctcaggcacaggagcagagaagactggcgtgtgccccgagctccaggctgaccagaactgcacgcaagagtgcgtctcggacagcgaatgcgccgacaacctcaagtgctgcagcgcgggctgtgccaccttctgctctctgcccaatgataaggagggttcctgcccccaggtgaacattaactttccccagctcggcctctgtcgggaccagtgccaggtggacagccagtgtcctggccagatgaaatgctgccgcaatggctgtgggaaggtgtcctgtgtcactcccaatttctgagctccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagcaaaaa

SEQ ID NO: 3 - protein, 79 aa, Homo sapiens
MPACRLGPLAAALLLSLLLFGFTLVSGTGAEKTGVCPELQADQNCTQECVSDSECADNLKCCSAGCATFCSLPNGQLAE

SEQ ID NO: 4 - DNA, 694 bp, Homo sapiens
cacctgcaccccgcccgggcatagcaccatgcctgcttgtcgcctaggcccgctagccgccgccctcctcctcagcctgctgctgttcggcttcaccctagtctcaggcacaggagcagagaagactggcgtgtgccccgagctccaggctgaccagaactgcacgcaagagtgcgtctcggacagcgaatgcgccgacaacctcaagtgctgcagcgcgggctgtgccaccttctgctctctgcccaatggccaactggctgagtgattcgaagaaagtgaggaatcctccctggacactgtatcgcccttcgtcgtctttcagtcaatctcttccactctaaggattgagtgagcgcgagctggggactctctcaaagataaggagggttcctgcccccaggtgaacattaactttccccagctcggcctctgtcgggaccagtgccaggtggacagccagtgtcctggccagatgaaatgctgccgcaatggctgtgggaaggtgtcctgtgtcactcccaatttctgaggtccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagc

SEQ ID NO: 5 - protein, 76 aa, Homo sapiens
MPACRLGPLAAALLLSLLLFGFTLVSDKEGSCPQVNINFPQLGLCRDQCQVDSQCPGQMKCCRNGCGKVSCVTPNF

SEQ ID NO: 6 - DNA, 421 bp, Homo sapiens
cacctgcaccccgcccgggcatagcaccatgcctgcttgtcgcctaggcccgctagccgccgccctcctcctcagcctgctgctgttcggcttcaccctagtctcagataaggagggttcctgcccccaggtgaacattaactttccccagctcggcctctgtcgggaccagtgccaggtggacagccagtgtcctggccagatgaaatgctgccgcaatggctgtgggaaggtgtcctgtgtcactcccaatttctgaggtccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagc

SEQ ID NO: 7 - protein, 102 aa, Homo sapiens
MPACRLGPLAAALLLSLLLFGFTLVSGTGAEKTGVCPELQADQNCTQECVSDSECADNLKCCSAGCATFCSLPNALFHWHLKTRRLWEISGPRPRRPTWDSS

SEQ ID NO: 8 - DNA, 358 bp, Homo sapiens
cacctgcaccccgcccgggcatagcaccatgcctgcttgtcgcctaggcccgctagccgccgccctcctcctcagcctgctgctgttcggcttcaccctagtctcaggcacaggagcagagaagactggcgtgtgccccgagctccaggctgaccagaactgcacgcaagagtgcgtctcggacagcgaatgcgccgacaacctcaagtgctgcagcgcgggctgtgccaccttctgctctctgcccaatgcactgttccactggcacctaaagacacggaggctctgggagatttctggccctaggccacgaaggcccacttgggactcaagctgaggtcctgtgattccatttggg

SEQ ID NO: 9 - protein, 73 aa, Homo sapiens
MLQVQVNLPVSPLPTYPYSFFYPDKEGSCPQVNINFPQLGLCRDQCQVDSQCPGQMKCCRNGCGKVSCVTPNF

SEQ ID NO: 10 - DNA, 411 bp, Homo sapiens
agcccagtgaggggcagtgggggggccatgctgcaggtacaagttaatctccctgtatcgcctctgcccacttacccttactcctttttctacccagataaggagggttcctgcccccaggtgaacattaactttccccagctcggcctctgtcgggaccagtgccaggtggacagccagtgtcctggccagatgaaatgctgccgcaatggctgtgggaaggtgtcctgtgtcactcccaatttctgaggtccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagc

SEQ ID NO: 11 - protein, 244 aa, Homo sapiens
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 12 - DNA, 1527 bp, Homo sapiens
ggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaa

SEQ ID NO: 13 - protein, 244 aa, Homo sapiens; identical to SEQ ID NO: 11

SEQ ID NO: 14 - DNA, 1527 bp, Homo sapiens
aggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatg

SEQ ID NO: 15 - protein, 137 aa, Homo sapiens
MLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 16 - DNA, 1495 bp, Homo sapiens
aggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaa

SEQ ID NO: 17 - protein, 40 aa, Homo sapiens
MKKLMVVLSLIAAGIFRSSWGSITFGKGRVPRSRVLLSGL

SEQ ID NO: 18 - DNA, 1386 bp, Homo sapiens
aggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcaggagcggccatgaagaagctgatggtggtgctgagtctgattgctgcaggaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaa

SEQ ID NO: 19 - protein, 276 aa, Homo sapiens
MRAPHLHLSAASGARALAKLLPLLMAQLWAAEAALLPQNDTRLDPEAYGSPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVLGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 20 - DNA, 1580 bp, Homo sapiens
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 21 - protein, 276 aa, Homo sapiens; identical to SEQ ID NO: 19

SEQ ID NO: 22 - DNA, 1443 bp, Homo sapiens
accagcggcagaccacaggcagggcagaggcacgtctgggtcccctccctccttcctatcggcgactcccaggatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 23 - protein, 180 aa, Homo sapiens
MLCLLLTLGVALVCGVPAMDIPQTKQDLELPKLAGTWHSMAMATNNISLMATLKAPLRVHITSLLPTPEDNLEIVLHRWENNSCVEKKVLGEKTENPKKFKINYTVANEATLLDTDYDNFLFLCLQDTTTPIQSMMCQYLARVLVEDDEIMQGFIRAFRPLPRHLWYLLDLKQMEEPCRF

SEQ ID NO: 24 - DNA, 857 bp, Homo sapiens
catccctctggctccagagctcagagccacccacagccgcagccatgctgtgcctcctgctcaccctgggcgtggccctggtctgtggtgtcccggccatggacatcccccagaccaagcaggacctggagctcccaaagttggcagggacctggcactccatggccatggcgaccaacaacatctccctcatggcgacactgaaggcccctctgagggtccacatcacctcactgttgcccacccccgaggacaacctggagatcgttctgcacagatgggagaacaacagctgtgttgagaagaaggtccttggagagaagactgagaatccaaagaagttcaagatcaactatacggtggcgaacgaggccacgctgctcgatactgactacgacaatttcctgtttctctgcctacaggacaccaccacccccatccagagcatgatgtgccagtacctggccagagtcctggtggaggacgatgagatcatgcagggattcatcagggctttcaggcccctgcccaggcacctatggtacttgctggacttgaaacagatggaagagccgtgccgtttctaggtgagctcctgcctggtcctgcctcctggctcacctccgcctccaggaagaccagactcccacccttccacacctccagagcagtgggacttcctcctgccctttcaaagaataaccacagctcagaagacgatgacgtggtcatctgtgtcgccatccccttcctgctgcacacctgcaccacggccatggggaggctgctccctgggggcagagtctctggcagaggttattaataaacccttggagcatgaaaaaaaaaaaaaaaaa

SEQ ID NO: 25 - protein, 180 aa, Homo sapiens; identical to SEQ ID NO: 23

SEQ ID NO: 26 - DNA, 828 bp, Homo sapiens
catccctctggctccagagctcagagccacccacagccgcagccatgctgtgcctcctgctcaccctgggcgtggccctggtctgtggtgtcccggccatggacatcccccagaccaagcaggacctggagctcccaaagttggcagggacctggcactccatggccatggcgaccaacaacatctccctcatggcgacactgaaggcccctctgagggtccacatcacctcactgttgcccacccccgaggacaacctggagatcgttctgcacagatgggagaacaacagctgtgttgagaagaaggtccttggagagaagactgagaatccaaagaagttcaagatcaactatacggtggcgaacgaggccacgctgctcgatactgactacgacaatttcctgtttctctgcctacaggacaccaccacccccatccagagcatgatgtgccagtacctggccagagtcctggtggaggacgatgagatcatgcagggattcatcagggctttcaggcccctgcccaggcacctatggtacttgctggacttgaaacagatggaagagccgtgccgtttctagctcacctccgcctccaggaagaccagactcccacccttccacacctccagagcagtgggacttcctcctgccctttcaaagaataaccacagctcagaagacgatgacgtggtcatctgtgtcgccatccccttcctgctgcacacctgcaccacggccatggggaggctgctccctgggggcagagtctctggcagaggttattaataaacccttggagcatgaaaaaaaaaaaaaaaaa

SEQ ID NO: 27 - protein, 402 aa, Homo sapiens [sequence omitted]

SEQ ID NO: 28 - DNA, 2876 bp, Homo sapiens
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 29 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 30 - DNA, 1209 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 31 - protein, 264 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 32 - DNA, 1182 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 33 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 34 - DNA, 1155 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 35 - protein, 96 aa, Homo sapiens
MTPGTQSPFFLLLLLTVLTVVTGSGHASSTPGGEKETSATQRSSVPSSTEKNAIPAPTTTKSCRETFLKCFCRFINKGVFWASPILSSGQDLWWYN

SEQ ID NO: 36 - DNA, 1136 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 37 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 38 - DNA, 1607 bp, Homo sapiens
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 39 - protein, 418 aa, Homo sapiens; identical to SEQ ID NO: 37

SEQ ID NO: 40 - DNA, 1588 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 41 - protein, 418 aa, Homo sapiens; identical to SEQ ID NO: 37

SEQ ID NO: 42 - DNA, 1902 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcgcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 43 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 44 - DNA, 1548 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggccaatgggagagctcttgttattattaatattgttgccgctgttgtgttgttgttattaattaatattcatattatttattttatacttacataaagattttgtaccagtgg

SEQ ID NO: 45 - protein, 281 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHERSLWGSWLPCKSTTALRPPCCEEAQATHV

SEQ ID NO: 46 - DNA, 1437 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaacgctcactctggggaagctggttgccatgtaaaagtactactgccctgagaccaccatgctgtgaggaagcccaagctactcatgtataaatgccatgtggagatagagccccagatgtttcagccatctcagcccaggcaccagacaagtgggtgaagaagccaccttggacatgtagccccagcagatgtgatatagagaagaaacaggaaacttggctatattagtttcctagggctgcctgtgataaattattacaaactttataaactaacacattgtgtgcctatatcaaaacatcatggaaggacaggcacagtggctcatgcctgtagtcctagcactttgggagggtgagaaaggaagatctcttgagctcaggagttcaagatcagcctgggcaacacagtgagacctcatctccactaaaaataaaaaaaaattggctgg

SEQ ID NO: 47 - protein, 290 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEVLELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 48 - DNA, 1360 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagtcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggccaatgggagagctcttgttattattaatattgttgccgctgttgtgttgttgttatta

SEQ ID NO: 49 - protein, 243 aa, Homo sapiens
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 50 - DNA, 1236 bp, Homo sapiens
ctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaaaaaaaaaaaaaaaaa

SEQ ID NO: 51 - protein, 366 aa, Homo sapiens
MVLHLLLFLLLTPQGGHSCQGLELARELVLAKVRALFLDALGPPAVTREGGDPGVRRLPRRHALGGFTHRGSEPEEEEDVSQAILFPATDASCEDKSAARGLAQEAEEGLFRYMFRPSQHTRSRQVTSAQLWFHTGLDRQGTAASNSSEPLLGLLALSPGGPVAVPMSLGHAPPHWAVLHLATSALSLLTHPVLVLLLRCPLCTCSARPEATPFLVAHTRTRPPSGGERARRSTPLMSWPWSPSALRLLQRPPEEPAAHANCHRVALNISFQELGWERWIVYPPSFIFHYCHGGCGLHIPPNLSLPVPGAPPTPAQPYSLLPGAQPCCAALPGTMRPLHVRTTSDGGYSFKYETVPNLLTQHCACI

SEQ ID NO: 52 - DNA, 1424 bp, Homo sapiens
gaaggactggggaagactggatgagaagggtagaagagggtgggtgtgggatggggaggggagagtggaaaggccctgggcagaccctggcagaaggggcacggggcagggtgtgagttccccactagcagggccaggtgagctatggtgctgcacctactgctcttcttgctgctgaccccacagggtgggcacagctgccaggggctggagctggcccgggaacttgttctggccaaggtgagggccctgttcttggatgccttggggccccccgcggtgaccagggaaggtggggaccctggagtcaggcggctgccccgaagacatgccctggggggcttcacacacaggggctctgagcccgaggaagaggaggatgtctcccaagccatccttttcccagccacagatgccagctgtgaggacaagtcagctgccagagggctggcccaggaggctgaggagggcctcttcagatacatgttccggccatcccagcatacacgcagccgccaggtgacttcagcccagctgtggttccacaccgggctggacaggcagggcacagcagcctccaatagctctgagcccctgctaggcctgctggcactgtcaccgggaggacccgtggctgtgcccatgtctttgggccatgctccccctcactgggccgtgctgcacctggccacctctgctctctctctgctgacccaccccgtcctggtgctgctgctgcgctgtcccctctgtacctgctcagcccggcctgaggccacgcccttcctggtggcccacactcggaccagaccacccagtggaggggagagagcccgacgctcaactcccctgatgtcctggccttggtctccctctgctctgcgcctgctgcagaggcctccggaggaaccggctgcccatgccaactgccacagagtagcactgaacatctccttccaggagctgggctgggaacggtggatcgtgtaccctcccagtttcatcttccactactgtcatggtggttgtgggctgcacatcccaccaaacctgtcccttccagtccctggggctccccctaccccagcccagccctactccttgctgccaggggcccagccctgctgtgctgctctcccagggaccatgaggcccctacatgtccgcaccacctcggatggaggttactctttcaagtatgagacagtgcccaaccttctcacgcagcactgtgcttgtatctaagggtggggggtcttccttcttaatcccatggctggtggccacgcccccaccatcatcagctgggaggaaaggcagagttgggaaatagatggctcccactcctccctcctttcacttctctgcctatgggctaccctccccaccccacttctatctcaataaagaacacagtgcatatg

SEQ ID NO: 53 - protein, 407 aa, Homo sapiens
MDGLPGRALGAACLLLLAAGWLGPEAWGSPTPPPTPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 54 - DNA, 3516 bp, Homo sapiens
ggatcctggagacaactttgccgtgtgacgcgccgggaggactgcagggcccgcggccgagggctcggcgccgcctgtgagcgggcccgcgcggccggctctcccgggcaccaagcttgctccgcgccactgcccgccggcccgcggcgaggacgacctgcccgtctccgccgccggcggcccttcctggcgcgaggcagtgagggcgaggcgctcaggtgcgagcgcggggccccgccgcagcgcccgccgcagcgccgcgccaagccgcgcccggctccgctccggggggctccagcgccttcgcttccgtctcagccaagttgcgtggacccgctctttcgccaccttccccagccgccggccgaaccgccgctcccactgacgctgctttcgcttcacccgaaccggggctgcggggcccccgacgcggaaaggatggggagaaggctgcagatgccgaggcgccccgagacgcccgtgcggcagtgacccgcgacctccgccccgcccggcgcgcccctcgggcccccggggccctcggcgccccttccctgccgcgcgggaacccccgaggcccggccggccccctccccctgcgagccggcggcagccctcccggcgggcgggcgggcggaggcccgggcgggcgcgggcgcgggcgggggcggggcggggcggcgcgcccggagcccggagcccggccctgcgctcggctcgactcggctcgcctcgcggcgggcgccctcgtcgccagcggcgcaccatggacgggctgcccggtcgggcgctgggggccgcctgccttctgctgctggcggccggctggctggggcctgaggcctggggctcacccacgcccccgccgacgcctgccgcgccgccgccacccccgccacccggagccccgggtggctcgcaggacacctgtacgtcgtgcggcggcttccggcggccagaggagctcggccgagtggacggcgacttcctggaggcggtgaagcggcacatcttgagccgcctgcagatgcggggccggcccaacatcacgcacgccgtgcctaaggccgccatggtcacggccctgcgcaagctgcacgcgggcaaggtgcgcgaggacggccgcgtggagatcccgcacctcgacggccacgccagcccgggcgccgacggccaggagcgcgtttccgaaatcatcagcttcgccgagacagatggcctcgcctcctcccgggtccgcctatacttcttcatctccaacgaaggcaaccagaacctgtttgtggtccaggccagcctgtggctttacctgaaactcctgccctacgtcctggagaagggcagccggcggaaggtgcgggtcaaagtgtacttccaggagcagggccacggtgacaggtggaacatggtggagaagagggtggacctcaagcgcagcggctggcataccttcccactcacggaggccatccaggccttgtttgagcggggcgagcggcgactcaacctagacgtgcagtgtgacagctgccaggagctggccgtggtgccggtgttcgtggacccaggcgaagagtcgcaccgaccctttgtggtggtgcaggctcggctgggcgacagcaggcaccgcattcgcaagcgaggcctggagtgcgatggccggaccaacctctgttgcaggcaacagttcttcattgacttccgcctcatcggctggaacgactggatcatagcacccaccggctactacggcaactactgtgagggcagctgcccagcctacctggcaggggtccccggctctgcctcctccttccacacggctgtggtgaaccagtaccgcatgcggggtctgaaccccggcacggtgaactcctgctgcattcccaccaagctgagcaccatgtccatgctgtacttcgatgatgagtacaacatcgtcaagcgggacgtgcccaacatgattgtggaggagtgcggctgcgcctgacagtgcaaggcaggggcacggtggtggggcacggagggcagtcccgggtgggcttcttccagccccccgcgggaacggggtacacggtgggctgagtacagtcattctgttgggctgtggagatagtgccagggtgcggcctgagatatttttctacagcttcatagagcaaccagtcaaaaccagagcgagaaccctcaactgacatgaaatactttaaaatgcacacgtagccacgcacagccagacgcatcctgccacccacacagcagcctccaggataccagcaaatggatgcggtgacaaatggcagcttagctacaaatgcctgtcagtcggagagaatggggtgagcagccaccattccaccagctggcccggccacgtctcgaagttgcgccttcccgagcacacataaaagcacaaagacagagacgcagagagagagagagagccacggagaggaaaagcagatgcaggggtggggagcgcagctcggcggaggctgcgtgtgccccgtggcttttaccaggcctgctctgcctggctcgatgtctgcttcttcccagcctgggatccttcgtgcttcaaggcctggggagcctgtccttccatgcccttgtcgagggaaagagacccagaaaggacacaacccgtcagagacctgggagcaggggcaatgaccgtttgactgtttgtggcttgggcctctgacatgacttatgtgtgtgtgtgtttttggggtggggagggagggagagaagagggggctaaatttgatgctttaactgatctccaacagttgacaggtcatccttgccagttgtataactgaaaaaggacttttctaccaggtatgaccttttaagtgaaaatctgaattgttctaaatggaaagaaaaaaagttgcaatctgtgcccttcattggggacattcctctaggactggtttggggacgggtgggaatgacccctaggcaaggggatgagaccgcaggaggaaatggcggggaggtggcattcttgaactgctgaggatggggggtgtcccctcagcggaggccaagggaggggagcagcctagttggtcttggagagatggggaaggctttcagctgatttgcagaagttgcccatgtgggcccaaccatcagggctggccgtggacgtggcccctgcccactcacctgcccgcctgcccgcccgcccgcatagcacttgcagacctgcctgaacgcacatgacatagcacttgccgatctgcgtgtgcccagaagtggcccttggccgagcgccgaactcgctcgccctctagatgtccaagtgccacgtgaactatgcaatttaaagggttgacccacactagacgaaactggactcgtacgactctttttatattttttatacttgaaatgaaatcctttgcttcttttttaagcgaatgattgcttttaatgtttgcactgatttagttgcatgattagtcagaaactgccatttgaaaaaaaagttatttttatagcagc

SEQ ID NO: 55 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 56 - DNA, 2175 bp, Homo sapiens
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaa

SEQ ID NO: 57 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 58 - DNA, 43816 bp, Homo sapiens
aagcgttgcacaattcccccaacctccatacatacggcagctcttctagacacaggttttcccaggtcaaatgcggggaccccagccatatctcccaccctgagaaattttggagtttcagggagctcagaagctctgcagaggccaccctctctgaggggattcttcttagacctccatccagaggcaaatgttgacctgtccatgctgaaaccctcaggccttcctgggtcatcttctcccacccgctccttgatgacagggagcaggagcactaaagccacaccagaaatggattcaggactgacaggagccaccttgtcacctaagacatctacaggtgcaatcgtggtgacagaacatactctgccctttacttccccagataagaccttggccagtcctacatcttcggttgtgggaagaaccacccagtctttgggggtgatgtcctctgctctccctgagtcaacctctagaggaatgacacactccgagcaaagaaccagcccatcgctgagtccccaggtcaatggaactccctctaggaactaccctgctacaagcatggtttcaggattgagttccccaaggaccaggaccagttccacagaaggaaattttaccaaagaagcatctacatacacactcactgtagagaccacaagtggcccagtcactgagaagtacacagtccccactgagacctcaacaactgaaggtgacagcacagagaccccctgggacacaagatatattcctgtaaaaatcacatctccaatgaaaacatttgcagattcaactgcatccaaggaaaatgccccagtgtctatgactccagctgagaccacagttactgactcacatactccaggaaggacaaacccatcatttgggacactttattcttccttccttgacctatcacctaaagggaccccaaattccagaggtgaaacaagcctggaactgattctatcaaccactggatatcccttctcctctcctgaacctggctctgcaggacacagcagaataagtaccagtgcgcctttgtcatcatctgcttcagttctcgataataaaatatcagagaccagcatattctcaggccagagtctcacctcccctctgtctcctggggtgcccgaggccagagccagcacaatgcccaactcagctatccctttttccatgacactaagcaatgcagaaacaagtgccgaaagggtcagaagcacaatttcctctctggggactccatcaatatccacaaagcagacagcagagactatccttaccttccatgccttcgctgagaccatggatatacccagcacccacatagccaagactttggcttcagaatggttgggaagtccaggtacccttggtggcaccagcacttcagcgctgacaaccacatctccatctaccactttagtctcagaggagaccaacacccatcactccacgagtggaaaggaaacagaaggaactttgaatacatctatgactccacttgagacctctgctcctggagaagagtccgaaatgactgccaccttggtccccactctaggttttacaactcttgacagcaagatcagaagtccatctcaggtctcttcatcccacccaacaagagagctcagaaccacaggcagcacctctgggaggcagagttccagcacagctgcccacgggagctctgacatcctgagggcaaccacttccagcacctcaaaagcatcatcatggaccagtgaaagcacagctcagcaatttagtgaaccccagcacacacagtgggtggagacaagtcctagcatgaaaacagagagacccccagcatcaaccagtgtggcagcccctatcaccacttctgttccctcagtggtctctggcttcaccaccctgaagaccagctccacaaaagggatttggcttgaagaaacatctgcagacacactcatcggagaatccacagctggcccaaccacccatcagtttgctgttcccactgggatttcaatgacaggaggcagcagcaccaggggaagccagggcacaacccacctactcaccagagccacagcatcatctgagacatccgcagatttgactctggccacgaacggtgtcccagtctccgtgtctccagcagtgagcaagacggctgctggctcaagtcctccaggagggacaaagccatcatatacaatggtttcttctgtcatccctgagacatcatctctacagtcctcagctttcagggaaggaaccagcctgggactgactccattaaacactagacatcccttctcttcccctgaaccagactctgcaggacacaccaagataagcaccagcattcctctgttgtcatctgcttcagttcttgaggataaagtgtcagcgaccagcacattctcacaccacaaagccacctcatctattaccacagggactcctgaaatctcaacaaagacaaagcccagctcagccgttctttcctccatgaccctaagcaatgcagcaacaagtcctgaaagagtcagaaatgcaacttcccctctgactcatccatctccatcaggggaagagacagcagggagtgtcctcactctcagcacctctgctgagactacagactcacctaacatccacccaactgggacactgacttcagaatcgtcagagagtcctagcactctcagcctcccaagtgtctctggagtcaaaaccacattttcttcatctactccttccactcatctatttactagtggagaagaaacagaggaaacttcgaatccatctgtgtctcaacctgagacttctgtttccagagtaaggaccaccttggccagcacctctgtccctaccccagtattccccaccatggacacctggcctacacgttcagctcagttctcttcatcccacctagtgagtgagctcagagctacgagcagtacctcagttacaaactcaactggttcagctcttcctaaaatatctcacctcactgggacggcaacaatgtcacagaccaatagagacacgtttaatgactctgctgcaccccaaagcacaacttggccagagactagtcccagattcaagacagggttaccttcagcaacaaccactgtttcaacctctgccacttctctctctgctactgtaatggtctctaaattcacttctccagcaactagttccatggaagcaacttctatcagggaaccatcaacaaccatcctcacaacagagaccacgaatggcccaggctctatggctgtggcttctaccaacatcccaattggaaagggctacattactgaaggaagattggacacaagccatctgcccattggaaccacagcttcctctgagacatctatggattttaccatggccaaagaaagtgtctcaatgtcagtatctccatctcagtccatggatgctgctggctcaagcactccaggaaggacaagccaattcgttgacacattttctgatgatgtctatcatttaacatccagagaaattacaatacctagagatggaacaagctcagctctgactccacaaatgactgcaactcaccctccatctcctgatcctggctctgctagaagcacctggcttggcatcttgtcctcatctccttcttctcctactcccaaagtcacaatgagctccacattttcaactcagagagtcaccacaagcatgataatggacacagttgaaactagtcggtggaacatgcccaacttaccttccacgacttccttgacaccaagtaatattccaacaagtggtgccataggaaaaagcaccctggttcccttggacactccatctccagccacatcattggaggcatcagaagggggacttccaaccctcagcacctaccctgaatcaacaaacacacccagcatccacctcggagcacacgctagttcagaaagtccaagcaccatcaaacttaccatggcttcagtagtaaaacctggctcttacacacctctcaccttcccctcaatagagacccacattcatgtatcaacagccagaatggcttactcttctgggtcttcacctgagatgacagctcctggagagactaacactggtagtacctgggaccccaccacctacatcaccactacggatcctaaggatacaagttcagctcaggtctctacaccccactcagtgaggacactcagaaccacagaaaaccatccaaagacagagtccgccaccccagctgcttactctggaagtcctaaaatctcaagttcacccaatctcaccagtccggccacaaaagcatggaccatcacagacacaactgaacactccactcaattacattacacaaaattggcagaaaaatcatctggatttgagacacagtcagctccaggacctgtctctgtagtaatccctacctcccctaccattggaagcagcacattggaactaacttctgatgtcccaggggaacccctggtccttgctcccagtgagcagaccacaatcactctccccatggcaacatggctgagtaccagtttgacagaggaaatggcttcaacagaccttgatatttcaagtccaagttcacccatgagtacatttgctatttttccacctatgtccacaccttctcatgaactttcaaagtcagaggcagataccagtgccattagaaatacagattcaacaacgttggatcagcacctaggaatcaggagtttgggcagaactggggacttaacaactgttcctatcaccccactgacaaccacgtggaccagtgtgattgaacactcaacacaagcacaggacaccctttctgcaacgatgagtcctactcacgtgacacagtcactcaaagatcaaacatctataccagcctcagcatccccttcccatcttactgaagtctaccctgagctcgggacacaagggagaagctcctctgaggcaaccactttttggaaaccatctacagacacactgtccagagagattgagactggcccaacaaacattcaatccactccacccatggacaacacaacaacagggagcagtagtagtggagtcaccctgggcatagcccaccttcccataggaacatcctccccagctgagacatccacaaacatggcactggaaagaagaagttctacagccactgtctctatggctgggacaatgggactccttgttactagtgctccaggaagaagcatcagccagtcattaggaagagtttcctctgtcctttctgagtcaactactgaaggagtcacagattctagtaagggaagcagcccaaggctgaacacacagggaaatacagctctctcctcctctcttgaacccagctatgctgaaggaagccagatgagcacaagcatccctctaacctcatctcctacaactcctgatgtggaattcatagggggcagcacattttggaccaaggaggtcaccacagttatgacctcagacatctccaagtcttcagcaaggacagagtccagctcagctacccttatgtccacagctttgggaagcactgaaaatacaggaaaagaaaaactcagaactgcctctatggatcttccatctccaactccatcaatggaggtgacaccatggatttctctcactctcagtaatgcccccaataccacagattcacttgacctcagccatggggtgcacaccagctctgcagggactttggccactgacaggtcattgaatactggtgtcactagagcctccagattggaaaacggctctgatacctcttctaagtccctgtctatgggaaacagcactcacacttccatgacttacacagagaagagtgaagtgtcttcttcaatccatccccgacctgagacctcagctcctggagcagagaccactttgacttccactcctggaaacagggccataagcttaacattgcctttttcatccattccagtggaagaagtcatttctacaggcataacctcaggaccagacatcaactcagcacccatgacacattctcccatcaccccaccaacaattgtatggaccagtacaggcacaattgaacagtccactcaaccactacatgcagtttcttcagaaaaagtttctgtgcagacacagtcaactccatatgtcaactctgtggcagtgtctgcttcccctacccatgagaattcagtctcttctggaagcagcacatcctctccatattcctcagcctcacttgaatccttggattccacaatcagtaggaggaatgcaatcacttcctggctatgggacctcactacatctctccccactacaacttggccaagtactagtttatctgaggcactgtcctcaggccattctggggtttcaaacccaagttcaactacgactgaatttccactcttttcagctgcatccacatctgctgctaagcaaagaaatccagaaacagagacccatggtccccagaatacagccgcgagtactttgaacactgatgcatcctcggtcacaggtctttctgagactcctgtgggggcaagtatcagctctgaagtccctcttccaatggccataacttctagatcagatgtttctggccttacatctgagagtactgctaacccgagtttaggcacagcctcttcagcagggaccaaattaactaggacaatatccctgcccacttcagagtctttggtttcctttagaatgaacaaggatccatggacagtgtcaatccctttggggtcccatccaactactaatacagaaacaagcatcccagtaaacagcgcaggtccacctggcttgtccacagtagcatcagatgtaattgacacaccttcagatggggctgagagtattcccactgtctccttttccccctcccctgatactgaagtgacaactatctcacatttcccagaaaagacaactcattcatttagaaccatttcatctctcactcatgagttgacttcaagagtgacacctattcctggggattggatgagttcagctatgtctacaaagcccacaggagccagtccctccattacactgggagagagaaggacaatcacctctgctgctccaaccacttcccccatagttctcactgctagtttcacagagaccagcacagtttcactggataatgaaactacagtaaaaacctcagatatccttgacgcacggaaaacaaatgagctcccctcagatagcagttcttcttctgatctgatcaacacctccatagcttcttcaactatggatgtcactaaaacagcctccatcagtcccactagcatctcaggaatgacagcaagttcctccccatctctcttctcttcagatagaccccaggttcccacatctacaacagagacaaatacagccacctctccatctgtttccagtaacacctattctcttgatgggggctccaatgtgggtggcactccatccactttaccaccctttacaatcacccaccctgtcgagacaagctcggccctattagcctggtctagaccagtaagaactttcagcaccatggtcagcactgacactgcctccggagaaaatcctacctctagcaattctgtggtgacttctgttccagcaccaggtacatggaccagtgtaggcagtactactgacttacctgccatgggctttctcaagacaagtcctgcaggagaggcacactcacttctagcatcaactattgaaccagccactgccttcactccccatctctcagcagcagtggtcactggatccagtgctacatcagaagccagtcttctcactacgagtgaaagcaaagccattcattcttcaccacagaccccaactacacccacctctggagcaaactgggaaacttcagctactcctgagagccttttggtagtcactgagacttcagacacaacacttacctcaaagattttggtcacagataccatcttgttttcaactgtgtccacgccaccttctaaatttccaagtacggggactctgtctggagcttccttccctactttactcccggacactccagccatccctctcactgccactgagccaacaagttcattagctacatcctttgattccaccccactggtgactatagcttcggatagtcttggcacagtcccagagactaccctgaccatgtcagagacctcaaatggtgatgcactggttcttaagacagtaagtaacccagataggagcatccctggaatcactatccaaggagtaacagaaagtccactccatccttcttccacttccccctctaagattgttgctccacggaatacaacctatgaaggttcgatcacagtggcactttctactttgcctgcgggaactactggttcccttgtattcagtcagagttctgaaaactcagagacaacggctttggtagactcatcagctgggcttgagagggcatctgtgatgccactaaccacaggaagccagggtatggctagctctggaggaatcagaagtgggtccactcactcaactggaaccaaaacattttcttctctccctctgaccatgaacccaggtgaggttacagccatgtctgaaatcaccacgaacagactgacagctactcaatcaacagcacccaaagggatacctgtgaagcccaccagtgctgagtcaggcctcctaacacctgtctctgcctcctcaagcccatcaaaggcctttgcctcactgactacagctcccccaacttgggggatcccacagtctaccttgacatttgagttttctgaggtcccaagtttggatactaagtccgcttctttaccaactcctggacagtccctgaacaccattccagactcagatgcaagcacagcatcttcctcactgtccaagtctccagaaaaaaacccaagggcaaggatgatgacttccacaaaggccataagtgcaagctcatttcaatcaacaggttttactgaaacccctgagggatctgcctccccttctatggcagggcatgaacccagagtccccacttcaggaacaggggaccctagatatgcctcagagagcatgtcttatccagacccaagcaaggcatcatcagctatgacatcgacctctcttgcatcaaaactcacaactctcttcagcacaggtcaagcagcaaggtctggttctagttcctctcccataagcctatccactgagaaagaaacaagcttcctttcccccactgcatccacctccagaaagacttcactatttcttgggccttccatggcaaggcagcccaacatattggtgcatcttcagacttcagctctgacactttctccaacatccactctaaatatgtcccaggaggagcctcctgagttaacctcaagccagaccattgcagaagaagagggaacaacagctgaaacacagacgttaaccttcacaccatctgagaccccaacatccttgttacctgtctcttctcccacagaacccacagccagaagaaagagttctccagaaacatgggcaagctctatttcagttcctgccaagacctccttggttgaaacaactgatggaacgctagtgaccaccataaagatgtcaagccaggcagcacaaggaaattccacgtggcctgccccagcagaggagacggggagcagtccagcaggcacatccccaggaagcccagaaatgtctaccactctcaaaatcatgagctccaaggaacccagcatcagcccagagatcaggtccactgtgagaaattctccttggaagactccagaaacaactgttcccatggagaccacagtggaaccagtcacccttcagtccacagccctaggaagtggcagcaccagcatctctcacctgcccacaggaaccacatcaccaaccaagtcaccaacagaaaatatgttggctacagaaagggtctccctctccccatccccacctgaggcttggaccaacctttattctggaactccaggagggaccaggcagtcactggccacaatgtcctctgtctccctagagtcaccaactgctagaagcatcacagggactggtcagcaaagcagtccagaactggtttcaaagacaactggaatggaattctctatgtggcatggctctactggagggaccacaggggacacacatgtctctctgagcacatcttccaatatccttgaagaccctgtaaccagcccaaactctgtgagctcattgacagataaatccaaacataaaaccgagacatgggtaagcaccacagccattccctccactgtcctgaataataagataatggcagctgaacaacagacaagtcgatctgtggatgaggcttattcatcaactagttcttggtcagatcagacatctgggagtgacatcacccttggtgcatctcctgatgtcacaaacacattatacatcacctccacagcacaaaccacctcactagtgtctctgccctctggagaccaaggcattacaagcctcaccaatccctcaggaggaaaaacaagctctgcgtcatctgtcacatctccttcaatagggcttgagactctgagggccaatgtaagtgcagtgaaaagtgacattgcccctactgctgggcatctatctcagacttcatctcctgcggaagtgagcatcctggacgtaaccacagctcctactccaggtatctccaccaccatcaccaccatgggaaccaactcaatctcaactaccacacccaacccagaagtgggtatgagtaccatggacagcaccccggccacagagaggcgcacaacttctacagaacacccttccacctggtcttccacagctgcatcagattcctggactgtcacagacatgacttcaaacttgaaagttgcaagatctcctggaacaatttccacaatgcatacaacttcattcttagcctcaagcactgaattagactccatgtctactccccatggccgtataactgtcattggaaccagcctggtcactccatcctctgatgcttcagctgtaaagacagagaccagtacaagtgaaagaacattgagtccttcagacacaactgcatctactcccatctcaactttttctcgtgtccagaggatgagcatctcagttcctgacattttaagtacaagttggactcccagtagtacagaagcagaagatgtgcctgtttcaatggtttctacagatcatgctagtacaaagactgacccaaatacgcccctgtccacttttctgtttgattctctgtccactcttgactgggacactgggagatctctgtcatcagccacagccactacctcagctcctcagggggccacaactccccaggaactcactttggaaaccatgatcagcccagctacctcacagttgcccttctctatagggcacattacaagtgcagtcacaccagctgcaatggcaaggagctctggagttactttttcaagaccagatcccacaagcaaaaaggcagagcagacttccactcagcttcccaccaccacttctgcacatccagggcaggtgcccagatcagcagcaacaactctggatgtgatcccacacacagcaaaaactccagatgcaacttttcagagacaagggcagacagctcttacaacagaggcaagagctacatctgactcctggaatgagaaagaaaaatcaaccccaagtgcaccttggatcactgagatgatgaattctgtctcagaagataccatcaaggaggttaccagctcctccagtgtattaaggaccctgaatacgctggacataaacttggaatctgggacgacttcatccccaagttggaaaagcagcccatatgagagaattgccccttctgagtccaccacagacaaagaggcaattcacccttctacaaacacagtagagaccacaggctgggtcacaagttccgaacatgcttctcattccactatcccagcccactcagcgtcatccaaactcacatctccagtggttacaacctccaccagggaacaagcaatagtttctatgtcaacaaccacatggccagagtctacaagggctagaacagagcctaattccttcttgactattgaactgagggacgtcagcccttacatggacaccagctcaaccacacaaacaagtattatctcttccccaggttccactgcgatcaccaaggggcctagaacagaaattacctcctctaagagaatatccagctcattccttgcccagtctatgaggtcgtcagacagcccctcagaagccatcaccaggctgtctaactttcctgccatgacagaatctggaggaatgatccttgctatgcaaacaagtccacctggcgctacatcactaagtgcacctactttggatacatcagccacagcctcctggacagggactccactggctacgactcagagatttacatactcagagaagaccactctctttagcaaaggtcctgaggatacatcacagccaagccctccctctgtggaagaaaccagctcttcctcttccctggtacctatccatgctacaacctcgccttccaatattttgttgacatcacaagggcacagtccctcctctactccacctgtgacctcagttttcttgtctgagacctctggcctggggaagaccacagacatgtcgaggataagcttggaacctggcacaagtttacctcccaatttgagcagtacagcaggtgaggcgttatccacttatgaagcctccagagatacaaaggcaattcatcattctgcagacacagcagtgacgaatatggaggcaaccagttctgaatattctcctatcccaggccatacaaagccatccaaagccacatctccattggttacctcccacatcatgggggacatcacttcttccacatcagtatttggctcctccgagaccacagagattgagacagtgtcctctgtgaaccagggacttcaggagagaagcacatcccaggtggccagctctgctacagagacaagcactgtcattacccatgtgtctagtggtgatgctactactcatgtcaccaagacacaagccactttctctagcggaacatccatctcaagccctcatcagtttataacttctaccaacacatttacagatgtgagcaccaacccctccacctctctgataatgacagaatcttcaggagtgaccatcaccacccaaacaggtcctactggagctgcaacacagggtccatatctcttggacacatcaaccatgccttacttgacagagactccattagctgtgactccagattttatgcaatcagagaagaccactctcataagcaaaggtcccaaggatgtgtcctggacaagccctccctctgtggcagaaaccagctatccctcttccctgacacctttcttggtcacaaccatacctcctgccacttccacgttacaagggcaacatacatcctctcctgtttctgcgacttcagttcttacctctggactggtgaagaccacagatatgttgaacacaagcatggaacctgtgaccaattcacctcaaaatttgaacaatccatcaaatgagatactggccactttggcagccaccacagatatagagactattcatccttccataaacaaagcagtgaccaatatggggactgccagttcagcacatgtactgcattccactctcccagtcagctcagaaccatctacagccacatctccaatggttcctgcctccagcatgggggacgctcttgcttctatatcaatacctggttctgagaccacagacattgagggagagccaacatcctccctgactgctggacgaaaagagaacagcaccctccaggagatgaactcaactacagagtcaaacatcatcctctccaatgtgtctgtgggggctattactgaagccacaaaaatggaagtcccctcttttgatgcaacattcataccaactcctgctcagtcaacaaagttcccagatattttctcagtagccagcagtagactttcaaactctcctcccatgacaatatctacccacatgaccaccacccagacagggtcttctggagctacatcaaagattccacttgccttagacacatcaaccttggaaacctcagcagggactccatcagtggtgactgaggggtttgcccactcaaaaataaccactgcaatgaacaatgatgtcaaggacgtgtcacagacaaaccctccctttcaggatgaagccagctctccctcttctcaagcacctgtccttgtcacaaccttaccttcttctgttgctttcacaccgcaatggcacagtacctcctctcctgtttctatgtcctcagttcttacttcttcactggtaaagaccgcaggcaaggtggatacaagcttagaaacagtgaccagttcacctcaaagtatgagcaacactttggatgacatatcggtcacttcagcagccaccacagatatagagacaacgcatccttccataaacacagtagttaccaatgtggggaccaccggttcagcatttgaatcacattctactgtctcagcttacccagagccatctaaagtcacatctccaaatgttaccacctccaccatggaagacaccacaatttccagatcaatacctaaatcctctaagactacaagaactgagactgagacaacttcctccctgactcctaaactgagggagaccagcatctcccaggagatcacctcgtccacagagacaagcactgttccttacaaagagctcactggtgccactaccgaggtatccaggacagatgtcacttcctctagcagtacatccttccctggccctgatcagtccacagtgtcactagacatctccacagaaaccaacaccaggctgtctacctccccaataatgacagaatctgcagaaataaccatcaccacccaaacaggtcctcatggggctacatcacaggatacttttaccatggacccatcaaatacaaccccccaggcagggatccactcagctatgactcatggattttcacaattggatgtgaccactcttatgagcagaattccacaggatgtatcatggacaagtcctccctctgtggataaaaccagctccccctcttcctttctgtcctcacctgcaatgaccacaccttccctgatttcttctaccttaccagaggataagctctcctctcctatgacttcacttctcacctctggcctagtgaagattacagacatattacgtacacgcttggaacctgtgaccagctcacttccaaatttcagcagcacctcagataagatactggccacttctaaagacagtaaagacacaaaggaaatttttccttctataaacacagaagagaccaatgtgaaagccaacaactctggacatgaatcccattcccctgcactggctgactcagagacacccaaagccacaactcaaatggttatcaccaccactgtgggagatccagctccttccacatcaatgccagtgcatggttcctctgagactacaaacattaagagagagccaacatatttcttgactcctagactgagagagaccagtacctctcaggagtccagctttcccacggacacaagttttctactttccaaagtccccactggtactattactgaggtctccagtacaggggtcaactcttctagcaaaatttccaccccagaccatgataagtccacagtgccacctgacaccttcacaggagagatccccagggtcttcacctcctctattaagacaaaatctgcagaaatgacgatcaccacccaagcaagtcctcctgagtctgcatcgcacagtacccttcccttggacacatcaaccacactttcccagggagggactcattcaactgtgactcagggattcccatactcagaggtgaccactctcatgggcatgggtcctgggaatgtgtcatggatgacaactccccctgtggaagaaaccagctctgtgtcttccctgatgtcttcacctgccatgacatccccttctcctgtttcctccacatcaccacagagcatcccctcctctcctcttcctgtgactgcacttcctacttctgttctggtgacaaccacagatgtgttgggcacaacaagcccagagtctgtaaccagttcacctccaaatttgagcagcatcactcatgagagaccggccacttacaaagacactgcacacacagaagccgccatgcatcattccacaaacaccgcagtgaccaatgtagggacttccgggtctggacataaatcacaatcctctgtcctagctgactcagagacatcgaaagccacacctctgatgagtaccacctccaccctgggggacacaagtgtttccacatcaactcctaatatctctcagactaaccaaattcaaacagagccaacagcatccctgagccctagactgagggagagcagcacgtctgagaagaccagctcaacaacagagacaaatactgccttttcttatgtgcccacaggtgctattactcaggcctccagaacagaaatctcctctagcagaacatccatctcagaccttgatcggcccacaatagcacccgacatctccacaggaatgatcaccaggctcttcacctcccccatcatgacaaaatctgcagaaatgaccgtcaccactcaaacaactactcctggggctacatcacagggtatccttccctgggacacatcaaccacacttttccagggagggactcattcaaccgtgtctcagggattcccacactcagagataaccactcttcggagcagaacccctggagatgtgtcatggatgacaactccccctgtggaagaaaccagctctgggttttccctgatgtcaccttccatgacatccccttctcctgtttcctccacatcaccagagagcatcccctcctctcctctccctgtgactgcacttcttacttctgttctggtgacaaccacaaatgtattgggcacaacaagcccagagcccgtaacgagttcacctccaaatttaagcagccccacacaggagagactgaccacttacaaagacactgcgcacacagaagccatgcatgcttccatgcatacaaacactgcagtggccaacgtggggacctccatttctggacatgaatcacaatcttctgtcccagctgattcacacacatccaaagccacatctccaatgggtatcaccttcgccatgggggatacaagtgtttctacatcaactcctgccttctttgagactagaattcagactgaatcaacatcctctttgattcctggattaagggacaccaggacgtctgaggagatcaacactgtgacagagaccagcactgtcctttcagaagtgcccactactactactactgaggtctccaggacagaagttatcacttccagcagaacaaccatctcagggcctgatcattccaaaatgtcaccctacatctccacagaaaccatcaccaggctctccacttttccttttgtaacaggatccacagaaatggccatcaccaaccaaacaggtcctatagggactatctcacaggctacccttaccctggacacatcaagcacagcttcctgggaagggactcactcacctgtgactcagagatttccacactcagaggagaccactactatgagcagaagtactaagggcgtgtcatggcaaagccctccctctgtggaagaaaccagttctccttcttccccagtgcctttacctgcaataacctcacattcatctctttattccgcagtatcaggaagtagccccacttctgctctccctgtgacttcccttctcacctctggcaggaggaagaccatagacatgttggacacacactcagaacttgtgaccagctccttaccaagtgcaagtagcttctcaggtgagatactcacttctgaagcctccacaaatacagagacaattcacttttcagagaacacagcagaaaccaatatggggaccaccaattctatgcataaactacattcctctgtctcaatccactcccagccatccggacacacacctccaaaggttactggatctatgatggaggacgctattgtttccacatcaacacctggttctcctgagactaaaaatgttgacagagactcaacatcccctctgactcctgaactgaaagaggacagcaccgccctggtgatgaactcaactacagagtcaaacactgttttctccagtgtgtccctggatgctgctactgaggtctccagggcagaagtcacctactatgatcctacattcatgccagcttctgctcagtcaacaaagtccccagacatttcacctgaagccagcagcagtcattctaactctcctcccttgacaatatctacacacaagaccatcgccacacaaacaggtccttctggggtgacatctcttggccaactgaccctggacacatcaaccatagccacctcagcaggaactccatcagccagaactcaggattttgtagattcagaaacaaccagtgtcatgaacaatgatctcaatgatgtgttgaagacaagccctttctctgcagaagaagccaactctctctcttctcaggcacctctccttgtgacaacctcaccttctcctgtaacttccacattgcaagagcacagtacctcctctcttgtttctgtgacctcagtacccacccctacactggcgaagatcacagacatggacacaaacttagaacctgtgactcgttcacctcaaaatttaaggaacaccttggccacttcagaagccaccacagatacacacacaatgcatccttctataaacacagcagtggccaatgtggggaccaccagttcaccaaatgaattctattttactgtctcacctgactcagacccatataaagccacatccgcagtagttatcacttccacctcgggggactcaatagtttccacatcaatgcctagatcctctgcgatgaaaaagattgagtctgagacaactttctccctgatatttagactgagggagactagcacctcccagaaaattggctcatcctcagacacaagcacggtctttgacaaagcattcactgctgctactactgaggtctccagaacagaactcacctcctctagcagaacatccatccaaggcactgaaaagcccacaatgtcaccggacacctccacaagatctgtcaccatgctttctacttttgctggcctgacaaaatccgaagaaaggaccattgccacccaaacaggtcctcatagggcgacatcacagggtacccttacctgggacacatcaatcacaacctcacaggcagggacccactcagctatgactcatggattttcacaattagatttgtccactcttacgagtagagttcctgagtacatatcagggacaagcccaccctctgtggaaaaaaccagctcttcctcttcccttctgtctttaccagcaataacctcaccgtcccctgtacctactacattaccagaaagtaggccgtcttctcctgttcatctgacttcactccccacctctggcctagtgaagaccacagatatgctggcatctgtggccagtttacctccaaacttgggcagcacctcacataagataccgactacttcagaagacattaaagatacagagaaaatgtatccttccacaaacatagcagtaaccaatgtggggaccaccacttctgaaaaggaatcttattcgtctgtcccagcctactcagaaccacccaaagtcacctctccaatggttacctctttcaacataagggacaccattgtttccacatccatgcctggctcctctgagattacaaggattgagatggagtcaacattctccctggctcatgggctgaagggaaccagcacctcccaggaccccatcgtatccacagagaaaagtgctgtccttcacaagttgaccactggtgctactgagacctctaggacagaagttgcctcttctagaagaacatccattccaggccctgatcattccacagagtcaccagacatctccactgaagtgatccccagcctgcctatctcccttggcattacagaatcttcaaatatgaccatcatcactcgaacaggtcctcctcttggctctacatcacagggcacatttaccttggacacaccaactacatcctccagggcaggaacacactcgatggcgactcaggaatttccacactcagaaatgaccactgtcatgaacaaggaccctgagattctatcatggacaatccctccttctatagagaaaaccagcttctcctcttccctgatgccttcaccagccatgacttcacctcctgtttcctcaacattaccaaagaccattcacaccactccttctcctatgacctcactgctcacccctagcctagtgatgaccacagacacattgggcacaagcccagaacctacaaccagttcacctccaaatttgagcagtacctcacatgagatactgacaacagatgaagacaccacagctatagaagccatgcatccttccacaagcacagcagcgactaatgtggaaaccaccagttctggacatgggtcacaatcctctgtcctagctgactcagaaaaaaccaaggccacagctccaatggataccacctccaccatggggcatacaactgtttccacatcaatgtctgtttcctctgagactacaaaaattaagagagagtcaacatattccttgactcctggactgagagagaccagcatttcccaaaatgccagcttttccactgacacaagtattgttctttcagaagtccccactggtactactgctgaggtctccaggacagaagtcacctcctctggtagaacatccatccctggcccttctcagtccacagttttgccagaaatatccacaagaacaatgacaaggctctttgcctcgcccaccatgacagaatcagcagaaatgaccatccccactcaaacaggtccttctgggtctacctcacaggatacccttaccttggacacatccaccacaaagtcccaggcaaagactcattcaactttgactcagagatttccacactcagagatgaccactctcatgagcagaggtcctggagatatgtcatggcaaagctctccctctctggaaaatcccagctctctcccttccctgctgtctttacctgccacaacctcacctcctcccatttcctccacattaccagtgactatctcctcctctcctcttcctgtgacttcacttctcacctctagcccggtaacgaccacagacatgttacacacaagcccagaacttgtaaccagttcacctccaaagctgagccacacttcagatgagagactgaccactggcaaggacaccacaaatacagaagctgtgcatccttccacaaacacagcagcgtccaatgtggagattcccagctctggacatgaatccccttcctctgccttagctgactcagagacatccaaagccacatcaccaatgtttattacctccacccaggaggatacaactgttgccatatcaacccctcacttcttggagactagcagaattcagaaagagtcaatttcctccctgagccctaaattgagggagacaggcagttctgtggagacaagctcagccatagagacaagtgctgtcctttctgaagtgtccattggtgctactactgagatctccaggacagaagtcacctcctctagcagaacatccatctctggttctgctgagtccacaatgttgccagaaatatccaccacaagaaaaatcattaagttccctacttcccccatcctggcagaatcatcagaaatgaccatcaagacccaaacaagtcctcctgggtctacatcagagagtacctttacattagacacatcaaccactccctccttggtaataacccattcgactatgactcagagattgccacactcagagataaccactcttgtgagtagaggtgctggggatgtgccacggcccagctctctccctgtggaagaaacaagccctccatcttcccagctgtctttatctgccatgatctcaccttctcctgtttcttccacattaccagcaagtagccactcctcttctgcttctgtgacttcacttctcacaccaggccaagtgaagactactgaggtgttggacgcaagtgcagaacctgaaaccagttcacctccaagtttgagcagcacctcagttgaaatactggccacctctgaagtcaccacagatacggagaaaattcatcctttctcaaacacggcagtaaccaaagttggaacttccagttctggacatgaatccccttcctctgtcctacctgactcagagacaaccaaagccacatcggcaatgggtaccatctccattatgggggatacaagtgtttctacattaactcctgccttatctaacactaggaaaattcagtcagagccagcttcctcactgaccaccagattgagggagaccagcacctctgaagagaccagcttagccacagaagcaaacactgttctttctaaagtgtccactggtgctactactgaggtctccaggacagaagccatctcctttagcagaacatccatgtcaggccctgagcagtccacaatgtcacaagacatctccataggaaccatccccaggatttctgcctcctctgtcctgacagaatctgcaaaaatgaccatcacaacccaaacaggtccttcggagtctacactagaaagtacccttaatttgaacacagcaaccacaccctcttgggtggaaacccactctatagtaattcagggatttccacacccagagatgaccacttccatgggcagaggtcctggaggtgtgtcatggcctagccctccctttgtgaaagaaaccagccctccatcctccccgctgtctttacctgccgtgacctcacctcatcctgtttccaccacattcctagcacatatccccccctctccccttcctgtgacttcacttctcacctctggcccggcgacaaccacagatatcttgggtacaagcacagaacctggaaccagttcatcttcaagtttgagcaccacctcccatgagagactgaccacttacaaagacactgcacatacagaagccgtgcatccttccacaaacacaggagggaccaatgtggcaaccaccagctctggatataaatcacagtcctctgtcctagctgactcatctccaatgtgtaccacctccaccatgggggatacaagtgttctcacatcaactcctgccttccttgagactaggaggattcagacagagctagcttcctccctgacccctggattgagggagtccagcggctctgaagggaccagctcaggcaccaagatgagcactgtcctctctaaagtgcccactggtgctactactgagatctccaaggaagacgtcacctccatcccaggtcccgctcaatccacaatatcaccagacatctccacaagaaccgtcagctggttctctacatcccctgtcatgacagaatcagcagaaataaccatgaacacccatacaagtcctttaggggccacaacacaaggcaccagtactttggacacgtcaagcacaacctctttgacaatgacacactcaactatatctcaaggattttcacactcacagatgagcactcttatgaggaggggtcctgaggatgtatcatggatgagccctccccttctggaaaaaactagaccttccttttctctgatgtcttcaccagccacaacttcaccttctcctgtttcctccacattaccagagagcatctcttcctctcctcttcctgtgacttcactcctcacgtctggcttggcaaaaactacagatatgttgcacaaaagctcagaacctgtaaccaactcacctgcaaatttgagcagcacctcagttgaaatactggccacctctgaagtcaccacagatacagagaaaactcatccttcttcaaacagaacagtgaccgatgtggggacctccagttctggacatgaatccacttcctttgtcctagctgactcacagacatccaaagtcacatctccaatggttattacctccaccatggaggatacgagtgtctccacatcaactcctggcttttttgagactagcagaattcagacagaaccaacatcctccctgacccttggactgagaaagaccagcagctctgaggggaccagcttagccacagagatgagcactgtcctttctggagtgcccactggtgccactgctgaagtctccaggacagaagtcacctcctctagcagaacatccatctcaggctttgctcagctcacagtgtcaccagagacttccacagaaaccatcaccagactccctacctccagcataatgacagaatcagcagaaatgatgatcaagacacaaacagatcctcctgggtctacaccagagagtactcatactgtggacatatcaacaacacccaactgggtagaaacccactcgactgtgactcagagattttcacactcagagatgaccactcttgtgagcagaagccctggtgatatgttatggcctagtcaatcctctgtggaagaaaccagctctgcctcttccctgctgtctctgcctgccacgacctcaccttctcctgtttcctctacattagtagaggatttcccttccgcttctcttcctgtgacttctcttctcaaccctggcctggtgataaccacagacaggatgggcataagcagagaacctggaaccagttccacttcaaatttgagcagcacctcccatgagagactgaccactttggaagacactgtagatacagaagacatgcagccttccacacacacagcagtgaccaacgtgaggacctccatttctggacatgaatcacaatcttctgtcctatctgactcagagacacccaaagccacatctccaatgggtaccacctacaccatgggggaaacgagtgtttccatatccacttctgacttctttgagaccagcagaattcagatagaaccaacatcctccctgacttctggattgagggagaccagcagctctgagaggatcagctcagccacagagggaagcactgtcctttctgaagtgcccagtggtgctaccactgaggtctccaggacagaagtgatatcctctaggggaacatccatgtcagggcctgatcagttcaccatatcaccagacatctctactgaagcgatcaccaggctttctacttcccccattatgacagaatcagcagaaagtgccatcactattgagacaggttctcctggggctacatcagagggtaccctcaccttggacacctcaacaacaaccttttggtcagggacccactcaactgcatctccaggattttcacactcagagatgaccactcttatgagtagaactcctggagatgtgccatggccgagccttccctctgtggaagaagccagctctgtctcttcctcactgtcttcacctgccatgacctcaacttcttttttctccacattaccagagagcatctcctcctctcctcatcctgtgactgcacttctcacccttggcccagtgaagaccacagacatgttgcgcacaagctcagaacctgaaaccagttcacctccaaatttgagcagcacctcagctgaaatattagccacgtctgaagtcaccaaagatagagagaaaattcatccctcctcaaacacacctgtagtcaatgtagggactgtgatttataaacatctatccccttcctctgttttggctgacttagtgacaacaaaacccacatctccaatggctaccacctccactctggggaatacaagtgtttccacatcaactcctgccttcccagaaactatgatgacacagccaacttcctccctgacttctggattaagggagatcagtacctctcaagagaccagctcagcaacagagagaagtgcttctctttctggaatgcccactggtgctactactaaggtctccagaacagaagccctctccttaggcagaacatccaccccaggtcctgctcaatccacaatatcaccagaaatctccacggaaaccatcactagaatttctactcccctcaccacgacaggatcagcagaaatgaccatcacccccaaaacaggtcattctggggcatcctcacaaggtacctttaccttggacacatcaagcagagcctcctggccaggaactcactcagctgcaactcacagatctccacactcagggatgaccactcctatgagcagaggtcctgaggatgtgtcatggccaagccgcccatcagtggaaaaaactagccctccatcttccctggtgtctttatctgcagtaacctcaccttcgccactttattccacaccatctgagagtagccactcatctcctctccgggtgacttctcttttcacccctgtcatgatgaagaccacagacatgttggacacaagcttggaacctgtgaccacttcacctcccagtatgaatatcacctcagatgagagtctggccacttctaaagccaccatggagacagaggcaattcagctttcagaaaacacagctgtgactcagatgggcaccatcagcgctagacaagaattctattcctcttatccaggcctcccagagccatccaaagtgacatctccagtggtcacctcttccaccataaaagacattgtttctacaaccatacctgcttcctctgagataacaagaattgagatggagtcaacatccaccctgacccccacaccaagggagaccagcacctcccaggagatccactcagccacaaagccaagcactgttccttacaaggcactcactagtgccacgattgaggactccatgacacaagtcatgtcctctagcagaggacctagccctgatcagtccacaatgtcacaagacatatccactgaagtgatcaccaggctctctacctcccccatcaagacagaatctacagaaatgaccattaccacccaaacaggttctcctggggctacatcaaggggtacccttaccttggacacttcaacaacttttatgtcagggacccactcaactgcatctcaaggattttcacactcacagatgaccgctcttatgagtagaactcctggagatgtgccatggctaagccatccctctgtggaagaagccagctctgcctctttctcactgtcttcacctgtcatgacctcatcttctcccgtttcttccacattaccagacagcatccactcttcttcgcttcctgtgacatcacttctcacctcagggctggtgaagaccacagagctgttgggcacaagctcagaacctgaaaccagttcacccccaaatttgagcagcacctcagctgaaatactggccatcactgaagtcactacagatacagagaaactggagatgaccaatgtggtaacctcaggttatacacatgaatctccttcctctgtcctagctgactcagtgacaacaaaggccacatcttcaatgggtatcacctaccccacaggagatacaaatgttctcacatcaacccctgccttctctgacaccagtaggattcaaacaaagtcaaagctctcactgactcctgggttgatggagaccagcatctctgaagagaccagctctgccacagaaaaaagcactgtcctttctagtgtgcccactggtgctactactgaggtctccaggacagaagccatctcttctagcagaacatccatcccaggccctgctcaatccacaatgtcatcagacacctccatggaaaccatcactagaatttctacccccctcacaaggaaagaatcaacagacatggccatcacccccaaaacaggtccttctggggctacctcgcagggtacctttaccttggactcatcaagcacagcctcctggccaggaactcactcagctacaactcagagatttccacagtcagtggtgacaactcctatgagcagaggtcctgaggatgtgtcatggccaagcccgctgtctgtggaaaaaaacagccctccatcttccctggtatcttcatcttcagtaacctcaccttcgccactttattccacaccatctgggagtagccactcctctcctgtccctgtcacttctcttttcacctctatcatgatgaaggccacagacatgttggatgcaagtttggaacctgagaccacttcagctcccaatatgaatatcacctcagatgagagtctggccgcttctaaagccaccacggagacagaggcaattcacgtttttgaaaatacagcagcgtcccatgtggaaaccaccagtgctacagaggaactctattcctcttccccaggcttctcagagccaacaaaagtgatatctccagtggtcacctcttcctctataagagacaacatggtttccacaacaatgcctggctcctctggcattacaaggattgagatagagtcaatgtcatctctgacccctggactgagggagaccagaacctcccaggacatcacctcatccacagagacaagcactgtcctttacaagatgccctctggtgccactcctgaggtctccaggacagaagttatgccctctagcagaacatccattcctggccctgctcagtccacaatgtcactagacatctccgatgaagttgtcaccaggctgtctacctctcccatcatgacagaatctgcagaaataaccatcaccacccaaacaggttattctctggctacatcccaggttacccttcccttgggcacctcaatgacctttttgtcagggacccactcaactatgtctcaaggactttcacactcagagatgaccaatcttatgagcaggggtcctgaaagtctgtcatggacgagccctcgctttgtggaaacaactagatcttcctcttctctgacatcattacctctcacgacctcactttctcctgtgtcctccacattactagacagtagcccctcctctcctcttcctgtgacttcacttatcctcccaggcctggtgaagactacagaagtgttggatacaagctcagagcctaaaaccagttcatctccaaatttgagcagcacctcagttgaaataccggccacctctgaaatcatgacagatacagagaaaattcatccttcctcaaacacagcggtggccaaagtgaggacctccagttctgttcatgaatctcattcctctgtcctagctgactcagaaacaaccataaccataccttcaatgggtatcacctccgctgtggacgataccactgttttcacatcaaatcctgccttctctgagactaggaggattccgacagagccaacattctcattgactcctggattcagggagactagcacctctgaagagaccacctcaatcacagaaacaagtgcagtcctttatggagtgcccactagtgctactactgaagtctccatgacagaaatcatgtcctctaatagaatacacatccctgactctgatcagtccacgatgtctccagacatcatcactgaagtgatcaccaggctctcttcctcatccatgatgtcagaatcaacacaaatgaccatcaccacccaaaaaagttctcctggggctacagcacagagtactcttaccttggccacaacaacagcccccttggcaaggacccactcaactgttcctcctagatttttacactcagagatgacaactcttatgagtaggagtcctgaaaatccatcatggaagagctctctctttgtggaaaaaactagctcttcatcttctctgttgtccttacctgtcacgacctcaccttctgtttcttccacattaccgcagagtatcccttcctcctctttttctgtgacttcactcctcaccccaggcatggtgaagactacagacacaagcacagaacctggaaccagtttatctccaaatctgagtggcacctcagttgaaatactggctgcctctgaagtcaccacagatacagagaaaattcatccttcttcaagcatggcagtgaccaatgtgggaaccaccagttctggacatgaactatattcctctgtttcaatccactcggagccatccaaggctacatacccagtgggtactccctcttccatggctgaaacctctatttccacatcaatgcctgctaattttgagaccacaggatttgaggctgagccattttctcatttgacttctggatttaggaagacaaacatgtccctggacaccagctcagtcacaccaacaaatacaccttcttctcctgggtccactcaccttttacagagttccaagactgatttcacctcttctgcaaaaacatcatccccagactggcctccagcctcacagtatactgaaattccagtggacataatcaccccctttaatgcttctccatctattacggagtccactgggataacctccttcccagaatccaggtttactatgtctgtaacagaaagtactcatcatctgagtacagatttgctgccttcagctgagactatttccactggcacagtgatgccttctctatcagaggccatgacttcatttgccaccactggagttccacgagccatctcaggttcaggtagtccattctctaggacagagtcaggccctggggatgctactctgtccaccattgcagagagcctgccttcatccactcctgtgccattctcctcttcaaccttcactaccactgattcttcaaccatcccagccctccatgagataacttcctcttcagctaccccatatagagtggacaccagtcttgggacagagagcagcactactgaaggacgcttggttatggtcagtactttggacacttcaagccaaccaggcaggacatcttcatcacccattttggataccagaatgacagagagcgttgagctgggaacagtgacaagtgcttatcaagttccttcactctcaacacggttgacaagaactgatggcattatggaacacatcacaaaaatacccaatgaagcagcacacagaggtaccataagaccagtcaaaggccctcagacatccacttcgcctgccagtcctaaaggactacacacaggagggacaaaaagaatggagaccaccaccacagctctgaagaccaccaccacagctctgaagaccacttccagagccaccttgaccaccagtgtctatactcccactttgggaacactgactcccctcaatgcatcaatgcaaatggccagcacaatccccacagaaatgatgatcacaaccccatatgttttccctgatgttccagaaacgacatcctcattggctaccagcctgggagcagaaaccagcacagctcttcccaggacaaccccatctgttttcaatagagaatcagagaccacagcctcactggtctctcgttctggggcagagagaagtccggttattcaaactctagatgtttcttctagtgagccagatacaacagcttcatgggttatccatcctgcagagaccatcccaactgtttccaagacaacccccaattttttccacagtgaattagacactgtatcttccacagccaccagtcatggggcagacgtcagctcagccattccaacaaatatctcacctagtgaactagatgcactgaccccactggtcactatttcggggacagatactagtacaacattcccaacactgactaagtccccacatgaaacagagacaagaaccacatggctcactcatcctgcagagaccagctcaactattcccagaacaatccccaatttttctcatcatgaatcagatgccacaccttcaatagccaccagtcctggggcagaaaccagttcagctattccaattatgactgtctcacctggtgcagaagatctggtgacctcacaggtcactagttctgggacagacagaaatatgactattccaactttgactctttctcctggtgaaccaaagacgatagcctcattagtcacccatcctgaagcacagacaagttcggccattccaacttcaactatctcgcctgctgtatcacggttggtgacctcaatggtcaccagtttggcggcaaagacaagtacaactaatcgagctctgacaaactcccctggtgaaccagctacaacagtttcattggtcacgcatcctgcacagaccagcccaacagttccctggacaacttccatttttttccatagtaaatcagacaccacaccttcaatgaccaccagtcatggggcagaatccagttcagctgttccaactccaactgtttcaactgaggtaccaggagtagtgacccctttggtcaccagttctagggcagtgatcagtacaactattccaattctgactctttctcctggtgaaccagagaccacaccttcaatggccaccagtcatggggaagaagccagttctgctattccaactccaactgtttcacctggggtaccaggagtggtgacctctctggtcactagttctagggcagtgactagtacaactattccaattctgactttttctcttggtgaaccagagaccacaccttcaatggccaccagtcatgggacagaagctggctcagctgttccaactgttttacctgaggtaccaggaatggtgacctctctggttgctagttctagggcagtaaccagtacaactcttccaactctgactctttctcctggtgaaccagagaccacaccttcaatggccaccagtcatggggcagaagccagctcaactgttccaactgtttcacctgaggtaccaggagtggtgacctctctggtcactagttctagtggagtaaacagtacaagtattccaactctgattctttctcctggtgaactagaaaccacaccttcaatggccaccagtcatggggcagaagccagctcagctgttccaactccaactgtttcacctggggtatcaggagtggtgacccctctggtcactagttccagggcagtgaccagtacaactattccaattctaactctttcttctagtgagccagagaccacaccttcaatggccaccagtcatggggtagaagccagctcagctgttctaactgtttcacctgaggtaccaggaatggtgacctctctggtcactagttctagagcagtaaccagtacaactattccaactctgactatttcttctgatgaaccagagaccacaacttcattggtcacccattctgaggcaaagatgatttcagccattccaactttagctgtctcccctactgtacaagggctggtgacttcactggtcactagttctgggtcagagaccagtgcgttttcaaatctaactgttgcctcaagtcaaccagagaccatagactcatgggtcgctcatcctgggacagaagcaagttctgttgttccaactttgactgtctccactggtgagccgtttacaaatatctcattggtcacccatcctgcagagagtagctcaactcttcccaggacaacctcaaggttttcccacagtgaattagacactatgccttctacagtcaccagtcctgaggcagaatccagctcagccatttcaacaactatttcacctggtataccaggtgtgctgacatcactggtcactagctctgggagagacatcagtgcaacttttccaacagtgcctgagtccccacatgaatcagaggcaacagcctcatgggttactcatcctgcagtcaccagcacaacagttcccaggacaacccctaattattctcatagtgaaccagacaccacaccatcaatagccaccagtcctggggcagaagccacttcagattttccaacaataactgtctcacctgatgtaccagatatggtaacctcacaggtcactagttctgggacagacaccagtataactattccaactctgactctttcttctggtgagccagagaccacaacctcatttatcacctattctgagacacacacaagttcagccattccaactctccctgtctcccctggtgcatcaaagatgctgacctcactggtcatcagttctgggacagacagcactacaactttcccaacactgacggagaccccatatgaaccagagacaacagccatacagctcattcatcctgcagagaccaacacaatggttcccaggacaactcccaagttttcccatagtaagtcagacaccacactcccagtagccatcaccagtcctgggccagaagccagttcagctgtttcaacgacaactatctcacctgatatgtcagatctggtgacctcactggtccctagttctgggacagacaccagtacaaccttcccaacattgagtgagaccccatatgaaccagagactacagccacgtggctcactcatcctgcagaaaccagcacaacggtttctgggacaattcccaacttttcccataggggatcagacactgcaccctcaatggtcaccagtcctggagtagacacgaggtcaggtgttccaactacaaccatcccacccagtataccaggggtagtgacctcacaggtcactagttctgcaacagacactagtacagctattccaactttgactccttctcctggtgaaccagagaccacagcctcatcagctacccatcctgggacacagactggcttcactgttccaattcggactgttccctctagtgagccagatacaatggcttcctgggtcactcatcctccacagaccagcacacctgtttccagaacaacctccagtttttcccatagtagtccagatgccacacctgtaatggccaccagtcctaggacagaagccagttcagctgtactgacaacaatctcacctggtgcaccagagatggtgacttcacagatcactagttctggggcagcaaccagtacaactgttccaactttgactcattctcctggtatgccagagaccacagccttattgagcacccatcccagaacagagacaagtaaaacatttcctgcttcaactgtgtttcctcaagtatcagagaccacagcctcactcaccattagacctggtgcagagactagcacagctctcccaactcagacaacatcctctctcttcaccctacttgtaactggaaccagcagagttgatctaagtccaactgcttcacctggtgtttctgcaaaaacagccccactttccacccatccagggacagaaaccagcacaatgattccaacttcaactctttcccttggtttactagagactacaggcttactggccaccagctcttcagcagagaccagcacgagtactctaactctgactgtttcccctgctgtctctgggctttccagtgcctctataacaactgataagccccaaactgtgacctcctggaacacagaaacctcaccatctgtaacttcagttggacccccagaattttccaggactgtcacaggcaccactatgaccttgataccatcagagatgccaacaccacctaaaaccagtcatggagaaggagtgagtccaaccactatcttgagaactacaatggttgaagccactaatttagctaccacaggttccagtcccactgtggccaagacaacaaccaccttcaatacactggctggaagcctctttactcctctgaccacacctgggatgtccaccttggcctctgagagtgtgacctcaagaacaagttataaccatcggtcctggatctccaccaccagcagttataaccgtcggtactggacccctgccaccagcactccagtgacttctacattctccccagggatttccacatcctccatccccagctccacagcagccacagtcccattcatggtgccattcaccctcaacttcaccatcaccaacctgcagtacgaggaggacatgcggcaccctggttccaggaagttcaacgccacagagagagaactgcagggtctgctcaaacccttgttcaggaatagcagtctggaatacctctattcaggctgcagactagcctcactcaggccagagaaggatagctcagccacggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatggtttcacccatcgaagctctatgcccaccaccagcactcctgggacctccacagtggatgtgggaacctcagggactccatcctccagccccagccccacgactgctggccctctcctgatgccgttcaccctcaacttcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccgagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatggtttcacccatcagagctctgtgtccaccaccagcactcctgggacctccacagtggatctcagaacctcagggactccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtctgagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatggtttcacccatcggacctctgtgcccaccagcagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggcccctacaccctggacaggaacagtctctatgtcaatggtttcacccattggatccctgtgcccaccagcagcactcctgggacctccacagtggaccttgggtcagggactccatcctccctccccagccccacaactgctggccctctcctggtgccgttcaccctcaacttcaccatcaccaacctgaagtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggtccctacaccctggacagaaacagtctctatgtcaatggtttcacccatcagacctctgcgcccaacaccagcactcctgggacctccacagtggaccttgggacctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaagagctgggcccctacaccctggacaggaacagtctctatgtcaatggtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagatgaccaatggcatcaaagagctgggcccctacaccctggaccggaacagtctctacgtcaatggtttcacccatcggagctctgggctcaccaccagcactccttggacttccacagttgaccttggaacctcagggactccatcccccgtccccagccccacaaccaccggccctctcctggtgccattcacactcaacttcaccatcactaacctacagtatgaggagaacatgggtcaccctggctccaggaagttcaacatcacggagagtgttctgcagggtctgctcaagcccttgttcaagagcaccagtgttggccctctgtattctggctgcagactgaccttgctcaggcctgagaaggatggagtagccaccagagtggacgccatctgcacccaccgccctgaccccaaaatccctgggctagacagacagcagctatactgggagctgagccagctgacccacagcatcactgagctgggaccctacaccctggatagggacagtctctatgtcaatggtttcacccagcggagctctgtgcccaccaccagcactcctgggactttcacagtacagccggaaacctctgagactccatcatccctccctggccccacagccactggccctgtcctgctgccattcaccctcaattttaccatcactaacctgcagtatgaggaggacatgcgtcgccctggctccaggaagttcaacaccacggagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacccatgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctgacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgcagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcctcatcggcttggcaggactcctgggagtcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcgga

SEQ ID NO: 59 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 60 - DNA, 1147 bp, Homo sapiens
accaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 61 - protein, 227 aa, Homo sapiens
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 62 - DNA, 1388 bp, Homo sapiens
gggatccttattctatatctcttggtatttagtgtaaaaattttaaaatctttacctagcaatcttgaggaagaaacttgataactgataatacatgagattgttacctaagtgaaatataatcctatatattcaacaaactttagagaaataagataaattttaaagtaaatgacttctgtagttttatagatcctccaaaccaatctagtctcagatctcaccttcatcatttctctcatttccttttggcctaattaatcaaaatccttcctagaatgttcatttctggccagtatgtcttcctgaatatgaataagaaataaaataccatttgatgtttgaaattatgggggtaatctcaatgacggaaatagatgaccaggaaaagggaaacgaatgcctgattcattatattcatgaagatatcaaaggtttataaagccaatatctgggaaagagaaaaccgtgagacttccagatcttctctggtgaagtgtgtttcctgcaacgatcacgaacatgaacatcaaaggatcgccatggaaagggtccctcctgctgctgctggtgtcaaacctgctcctgtgccagagcgtggcccccttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgctaagcccacatccatttcatctatttctgagaaggtccttaatgatccgttccattgcaagcttcttttagttgtatctcttttgaatccatgcttgggtgtaacaggtctcctcttaaaaaataaaaactgactccttagagacatcaaaatccaaaaaaaaaaaaaaaaaaaaaaaaaaaaaa

SEQ ID NO: 63 - protein, 132 aa, Homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 64 - DNA, 598 bp, Homo sapiens
cagagtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgcac

(19) United States
(12) Patent Application Publication
(10) Pub. No.: US 2009/0075307 A1
(43) Pub. Date: Mar. 19, 2009

(54) METHODS FOR IDENTIFYING PATIENTS WITH AN INCREASED LIKELIHOOD OF HAVING OVARIAN CANCER AND COMPOSITIONS THEREFOR

(75) Inventors: Timothy J. Fischer, Raleigh, NC (US); Douglas P. Malinowski, Hillsborough, NC (US); Qin He, Raleigh, NC (US); Clark M. Whitehead, Raleigh, NC (US); Robert L. Cheek, Mebane, NC (US); John W. Groelke, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: TriPath Imaging, Inc., Burlington, NC (US)

(21) Appl. No.: 12/261,330

(22) Filed: Oct. 30, 2008

Related U.S. Application Data
(63) Continuation of application No. 11/699,229, filed on Jan. 29, 2007.
(60) Provisional application No. 60/762,760, filed on Jan. 27, 2006.

(51) Int. Cl. G01N 33/53; C12Q 1/02; G01N 33/68; G01N 33/536

(52) U.S. Cl. 435/7.92; 435/29; 436/86; 436/536

(57) ABSTRACT

Screening methods for identifying patients with an increased likelihood of having ovarian cancer are provided. The screening methods involve the detection of expression of a plurality of biomarkers in a body sample, wherein overexpression of the biomarkers is indicative of an increased likelihood of having ovarian cancer. The screening methods may further comprise a two-step analysis. Biomarkers of interest include genes and proteins that are, for example, involved in defects in DNA replication/cell cycle control, cell growth and proliferation, escape from apoptosis, angiogenesis or lymphogenesis, or the mechanisms of cancer cell motility and invasion. In some aspects of the invention, expression of a biomarker is detected at the protein level using a biomarker-specific antibody or at the nucleic acid level using nucleic acid hybridization techniques. Methods for detecting ovarian cancer in patients are further disclosed herein. Kits for practicing the methods of the invention are further provided.

### CROSS REFERENCE TO RELATED APPLICATIONS

This application is a continuation of U.S. application Ser. No. 11/699,229, filed Jan. 29, 2007, which claims the benefit of U.S. Provisional Application Ser. No. 60/762,760, filed on Jan. 27, 2006, both of which are incorporated herein by reference in their entirety.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 364718SequenceListing.txt, a creation date of Oct. 29, 2008, and a size of 284 KB. The sequence listing filed via EFS-Web is part of the specification and is hereby incorporated in its entirety by reference herein.

### FIELD OF THE INVENTION

The present invention relates to methods and compositions for identifying women having an increased likelihood of having ovarian cancer.

### BACKGROUND OF THE INVENTION

Ovarian cancer represents a heterogeneous group of diseases that affect women on a global basis. There are several forms of ovarian cancer which include epithelial cancer, germ-line cancer of the ovaries and ovarian stromal cancer. Epithelial ovarian cancer represents the most common form of the disease. Approximately 5-10% of epithelial ovarian cancer represents a hereditary form of the disease and three common patterns are recognized: ovarian cancer alone; ovarian and breast cancer linked to BRAC1 and BRCA2 genetic linkage on chromosomes 17q21 and 13q12 respectively; and ovarian and colon cancer. The most important risk factor for ovarian cancer is a first degree relative with the disease (e.g., a mother, sister or daughter with ovarian cancer). See, for example, Patridge et al. (1999) CA-A Cancer Journal for Clinicians 49:297-320 In 2005, there were an estimated 22,000 new cases of ovarian cancer diagnoses and 16,000 deaths from ovarian cancer. See generally American Cancer Society website at www.cancer.org; National Cancer Institute website at www.cancer.gov. Ovarian cancer is a disease that primarily affects post-menopausal women with the median age for diagnosis at 63 years of age. However, the disease can affect women at all age groups. National Cancer Institute Surveillance, Epidemiology, and End Results (SEER) Program at www.seer.cancer.gov.

The classification of ovarian cancer stage is based upon the extent of localization versus spread of the disease beyond the ovaries. Stage 1 ovarian cancer is confined to one or both of the ovaries. Stage 2 disease involves a tumor in one or both ovaries with pelvic extension. In Stage 3 ovarian cancer, a tumor is present in or both ovaries with microscopically confirmed peritoneal metastasis outside the pelvis and/or regional lymph node metastasis. Stage 4 ovarian cancer is characterized by distant metastasis beyond the peritoneal cavity. Ovarian cancer is generally diagnosed in an advance stage of the disease due to the lack of specific clinical symptoms that would indicate the presence of small tumors. For women under the age of 50, less than 40% of ovarian cancers are detected when tumors are localized to one or both ovaries and when disease prognosis is best. For women over the age of 50, that number drops to less than 15%. Approximately 68% of women of all age groups afflicted with ovarian cancer are not diagnosed until distant metastasis is present. See generally National Cancer Institute Surveillance, Epidemiology, and End Results (SEER) Program at www.seer.cancer.gov.

Ovarian cancer spreads via local shedding from the ovarian epithelium into the peritoneal cavity followed by implantation on the peritoneum and local invasion of the bowel and bladder. The presence of lymph node involvement in ovarian cancer is evident in all stages of diagnosed ovarian cancer. The percentage of positive lymph nodes increases significantly with progression of the disease (i.e., Stage 1, 24%; Stage 2, 50%, Stage 3, 74%; Stage 4, 73%). Id. The survival of patients with ovarian cancer is a function of the stage at which the disease is diagnosed, with the 5-year survival decreasing with advanced disease. More than 90% of women diagnosed with ovarian cancer in Stage 1 survive for at least 5 years following diagnosis. The 5-year survival rate drops to less than 30% when the disease is not diagnosed until Stage 4 (i.e., distant metastasis). Id.

Epithelial ovarian cancer is the most common form of the disease. There are four recognized major histological classes of epithelial ovarian cancer and include serous, endometrioid, clear cell, and mucinous subtypes. The pathogenesis of ovarian cancer is poorly understood but is believed to arise from ovarian surface epithelium. See Bell (2005) Mod Pathol. 18 (Suppl 2):S19-32. Life factors that provide the greatest reduction in risk of ovarian cancer include multiparity, use of oral contraceptives, and breast feeding, all of which prevent ovulation. Because ovulation results in epithelial damage, followed by repair and possible inflammatory responses, repetition of this process throughout a woman's reproductive life without interruption appears to lead to cell damage and to increase the risk of ovarian cancer. See, for example, Ness et al. (1999) J. Natl. Cancer Inst. 91:1459-1467. However, there is no recognized, stepwise progression of ovarian cancer through defined precursor lesions, such as those recognized for both cervical carcinoma and colon cancer. Hence, considerable research has been directed at understanding the molecular basis for ovarian cancer and to understand the basic differences between the various histological subtypes of ovarian cancer. These studies have utilized gene expression analysis to provide this understanding and have identified a series of potential biomarkers for evaluation in diagnostic applications. See for example Ono et al. (2000) Cancer Res. 60:5007-11; Welsh et al. (2001) Proc. Natl. Acad. Sci. USA 98:1176-1181; Donninger et al. (2004) Oncogene 23:8065-8077; and Lee et al. (2004) Int. J. Oncol. 24(4):847-851.

Ovarian cancer is often detected with the presentation of overt clinical symptoms, most notably the presentation of abdominal pain, an adnexal mass, abdominal bloating, and urinary urgency. As such, the detection of ovarian cancer is often detected at an advanced stage, where the prognosis and clinical outcome is poor. Detection of ovarian cancer at an early stage (i.e., Stage 1) results in approximately 90% cure rate using standard surgery and chemotherapy; hence there is a clinical need to detect ovarian cancer at an early stage where treatment will be most effective. Unfortunately, current screening methods to detect early stage ovarian cancer are insufficient. The current practice for ovarian cancer screening employs the use of CA125 and transvaginal ultrasound (sonography). Rising serum levels of CA125 are associated with ovarian cancer and subsequent utilization of transvaginal ultrasound helps detect the presence of ovarian cancer. Confirmation of ovarian disease is based upon invasive procedures such as laprotomy. However, the use of CA125 is ineffective for general population screening due to issues of limited sensitivity, limited specificity, and a poor positive predictive value of <3%. Bast (2003) J Clin Oncol. 21(10 Suppl):200-205. As a result, there is no consensus on the recommendations for generally screening for ovarian cancer in the asymptomatic patient population. See National Cancer Institute Web Site at www.cancer.gov. For high risk patients, the generally accepted procedures for the detection of ovarian cancer include the use of pelvic examinations, the use of CA125 serum testing, and transvaginal ultrasound (sonography). Patridge et al. (1999) CA-A Cancer Journal for Clinicians 49:297-320.

CA125 is a well characterized tumor marker normally expressed on the surface of epithelial cells and is often detected in the serum of normal patients at 35 U/mL. Elevated serum levels of CA125 (>35 U/mL) are often detected in approximately 85% of ovarian cancer patients; the remaining 15% of ovarian cancer patients have normal serum levels of CA125. Furthermore, CA125 is elevated in only 50% of stage 1 ovarian cancer patients, thereby limiting its clinical utility in the early detection of ovarian cancer. However, elevated serum levels of CA125 are used for the monitoring of disease recurrence following therapeutic intervention and this represents the currently approved use for CA125 by the FDA. In addition, elevated serum levels of CA125 are predictive of future detectable ovarian cancer.

The low prevalence rates of ovarian cancer in the general population create significant challenges for the development of a screening test that would promote early detection of the disease. Screening methods for diseases with low prevalence rates such as ovarian cancer often result in a high ratio of false positives to true positives that limits the clinical utility of such screening programs. Given the significant risks associated with surgical exploration for possible ovarian cancer, a clinically useful screening test should refer to surgery no more than 10 women for every woman who actually has ovarian cancer (i.e., a positive predictive value (PPV) of at least 10%). Skates et al. (2004) J. Clin. Oncol. 22:4059-4066. PPV is highly dependent upon the prevalence rates for a particular disease or condition and will shift dramatically as a result of differences in disease prevalence. Therefore, with low-prevalence diseases, such as ovarian cancer, screening diagnostic tests with a relatively low PPV still have significant clinical utility. Potential ovarian cancer screening programs must be adjusted for the low prevalence of ovarian cancer and assessed for biomarker performance and clinical need. See, for example, Skates et al. (2004) J. Clin. Oncol. 22:4059-4066; Bast et al. (2005) Int. J. Gynecol. Cancer 15:274-281; and Rosen et al. (2005) Gyn. Oncol. 99:267-277. Despite efforts to identify a biomarker or panel of biomarkers for the detection, particularly early detection, of ovarian cancer, no adequate screening or diagnostic test that satisfies clinical needs currently exists. Currently available methods, such as detection of CA125, exhibit unacceptably high false-positive rates.

The current recommendations from the National Cancer Institute state that “there is insufficient evidence to establish that screening for ovarian cancer with serum markers such as CA125, transvaginal ultrasound or pelvic examinations would result in a decrease in mortality from ovarian cancer” (NCI Summary of Evidence (Level 4, 5); dated February 2005). In light of the serious risk of false-positives with currently available screening techniques, the NCI has not supported institution of general screening procedures for ovarian cancer. As such, no standardized screening test exists for ovarian cancer, despite the fact that early diagnosis significantly improves 5-year survival rates.

Therefore, a significant need exists in the art for reliable methods and compositions that are capable of specifically identifying women that have ovarian cancer. In particular, screening methods for identifying patients with an increased likelihood of having ovarian cancer are needed. Women identified as having an increased likelihood of having ovarian cancer could be selected for more aggressive diagnostic methods to definitively determine if they presently have the disease. Moreover, such screening methods could be performed in the general female patient population on a routine basis to facilitate the detection of ovarian cancer in the early stages of the disease when prognosis and disease outcome are best.

### BRIEF SUMMARY OF THE INVENTION

Screening methods for identifying patients with an increased likelihood of having ovarian cancer are provided. The methods of the invention generally comprise detecting in a patient body sample expression of a plurality of biomarkers that are selectively overexpressed in ovarian cancer. Overexpression of the biomarkers is indicative of an increased likelihood that the patient has ovarian cancer. The methods of the invention may comprise, for example, a “two-step” analysis, wherein a first assay step is performed to detect the expression of a first biomarker or panel of biomarkers. If the first biomarker or panel of biomarkers is overexpressed, a second assay step is performed to detect the expression of a second biomarker or panel of biomarkers. Overexpression of the first and second biomarkers or panels of biomarkers is indicative of an increased likelihood that the patient has ovarian cancer. The first assay step may be designed to enrich the patient population under review by eliminating a large percentage of women that are “true negatives.” The second assay step is typically intended to rule out those patients in the enriched population that do not presently have ovarian cancer by eliminating additional true negative patients from the enriched population. These assay steps may be performed as a single test encompassing both assay steps or as two distinct tests, wherein each test comprises one of the assay steps. A single biomarker or panel of biomarkers may be used in each analysis step to achieve the desired values for sensitivity, specificity, negative predictive value (NPV), and positive predictive value (PPV). Algorithms may be developed to combine particular biomarkers in the different assay steps to achieve the desired sensitivity, specificity, NPV, and PPV.

In other aspects of the invention, a screening method for identifying patients with an increased likelihood of having ovarian cancer comprises performing a “single-step” or “one-step” assay in which the overexpression of a plurality of biomarkers that are selectively overexpressed in ovarian cancer is assessed in a patient body sample. Overexpression of these biomarkers is indicative of an increased likelihood of the patient having ovarian cancer. In contrast to the two-step screening method described above, the single-step screening assay does not require performing a first assay step to detect overexpression of a particular biomarker(s) followed by a second assay step to assess if a second biomarker(s) is also overexpressed in order to identify a patient with an increased likelihood of having ovarian cancer.

A patient that is identified by the screening methods of the invention as having an increased likelihood of having ovarian cancer may be subjected to further diagnostic tests to definitively determine if the patient has ovarian cancer. Patients that are classified as having an increased likelihood of having ovarian cancer in accordance with the methods disclosed herein, but that are determined not to currently have the disease, are generally monitored on a regular basis for the development of ovarian cancer. Moreover, the present methods may be used to screen the general female patient population for ovarian cancer on a routine basis. Thus, the screening methods of the invention may permit the diagnosis of ovarian cancer at earlier stages of the disease when prognosis is significantly better. Kits for practicing the screening methods of the invention are also provided.

Biomarker expression can be assessed at the protein or nucleic acid level. In some embodiments, biomarker expression is detected at the protein level using biomarker-specific antibodies. Expression of the biomarkers of the invention can also be detected by nucleic acid-based techniques, including, for example, hybridization and RT-PCR. Biomarker expression can be assessed in a variety of body samples, including but not limited to blood (e.g., whole blood, blood serum, blood having platelets removed, etc.), lymph, ascitic fluids, urine, gynecological fluids (e.g., ovarian, fallopian, and uterine secretion, menses, etc.), biopsies, and fluids obtained during laparoscopy.

Methods for diagnosing ovarian cancer in a patient are also encompassed by the present invention. The diagnostic methods generally comprise detecting in a body sample the expression of a plurality of biomarkers that are selectively overexpressed in ovarian cancer. Overexpression of the plurality of biomarkers is indicative of the presence of ovarian cancer. Kits for practicing the diagnostic methods of the invention are also provided.

Methods for assessing the efficacy of a particular therapy for ovarian cancer in a patient are also disclosed herein. The invention is further directed to a method for monitoring the regression or progression of ovarian cancer in a patient.

### BRIEF DESCRIPTION OF THE DRAWINGS

FIG. 1 provides a schematic representation of an exemplary “two-step” screening test for identifying patients with an increased likelihood of having ovarian cancer. The 8 million female patients screened in this example represent the high-risk, asymptomatic U.S. patient population, as defined herein below. In the first assay step, a significant number of true negatives are eliminated from further testing, thereby leaving an enriched population for further analysis in the second assay step. The second assay step further rules out additional patients that are true negatives in order to identify those women with the highest risk of having ovarian cancer. Additional details regarding the two-step screening method are provided in the text.

FIG. 2 provides the Receiver Operating Characteristic (ROC) plots for HE4 obtained with samples from patients over the age of 55 (A) and with all patient samples. Additional experimental details are provided in Example 2.

FIG. 3 provides the ROC plots for inhibin A (INH) obtained with samples from patients over the age of 55 (A) and with all patient samples. Additional experimental details are provided in Example 2.

FIG. 4 provides the ROC plot for prolactin obtained with all patient samples. Additional experimental details are provided in Example 2.

FIG. 5 provides the ROC plot for PLAU-R obtained with all patient samples. Additional experimental details are provided in Example 2.

FIG. 6 provides the ROC plots for glycodelin (GLY) obtained with samples from patients over the age of 55 (A) and with all patient samples. Additional experimental details are provided in Example 2.

FIG. 7 provides the ROC plot for SLPI obtained with all patient samples. Additional experimental details are provided in Example 2.

FIG. 8 provides the ROC plot for CTHRC1 obtained with all patient samples. Additional experimental details are provided in Example 2.

FIG. 9 provides the ROC plot for PAI-1 obtained with all patient samples. Additional experimental details are provided in Example 2.

FIG. 10 provides the ROC plot for KLK-10 obtained with all patient samples. Additional experimental details are provided in Example 2.

FIG. 11 provides the ROC plots for CA125 obtained with samples from patients over the age of 55 (A) and with all patient samples. Additional experimental details are provided in Example 2.

FIG. 12 provides the ROC plot for KLK-6 obtained with all patient samples. Additional experimental details are provided in Example 2.

FIG. 13 provides the ROC plot for Muc-1 (MU) obtained with all patient samples. Additional experimental details are provided in Example 2.

FIG. 14 provides the ROC plot for MMP-7 (MM) obtained with all patient samples. Additional experimental details are provided in Example 2.

### DETAILED DESCRIPTION OF THE INVENTION

The present invention provides screening methods and compositions for identifying patients with ovarian cancer. The screening methods generally comprise detecting the expression of a plurality of biomarkers in a body sample, particularly a blood sample, more particularly a serum sample, from the patient. Overexpression of the biomarkers used in the practice of the invention is indicative of an increased likelihood of the presence of ovarian cancer. In particular screening methods of the invention, a two-step analysis is used to identify patients having an increased likelihood of having ovarian cancer. The first assay step is performed to detect the expression of a first biomarker or panel of biomarkers in a patient body sample. If the first biomarker or panel of biomarkers is determined to be overexpressed in the sample, a second assay step is performed to detect the expression of a second biomarker or panel of biomarkers. Overexpression of the first and second biomarkers or panels of biomarkers is indicative of an increased likelihood that the patient has ovarian cancer. In certain embodiments, antibodies are used to detect biomarker protein expression. In other aspects of the invention, when the expression of a panel of biomarkers is detected, overexpression of only a subset of the biomarkers analyzed may be sufficient to be indicative of an increased likelihood of the patient having ovarian cancer.

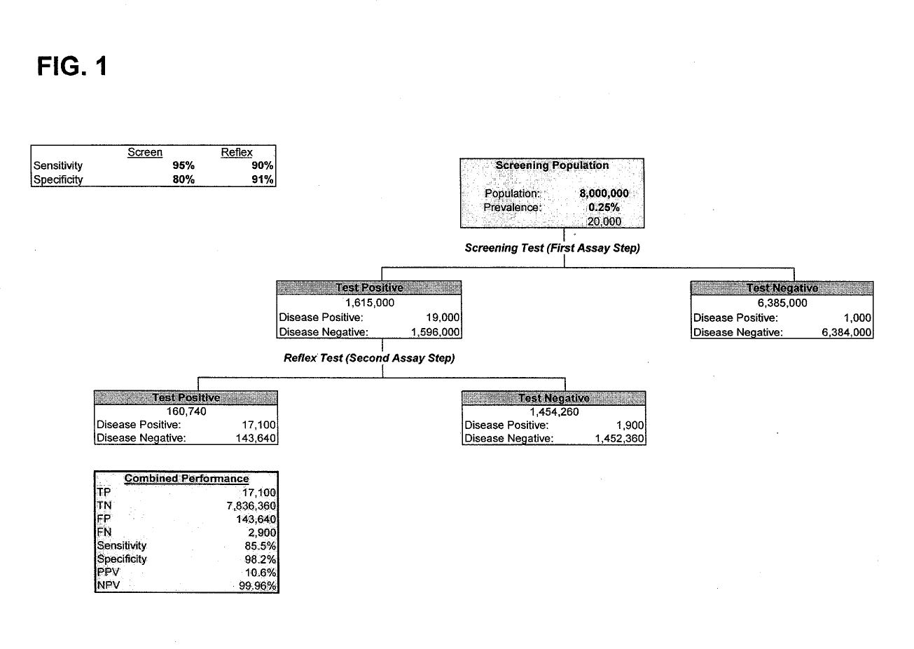

Although the invention is not limited to a particular mechanism, the first assay step is generally designed to enrich the true-positive patient population (i.e., on a percentage basis) by eliminating a large number of true negatives from further testing. The first assay step may employ a higher sensitivity and negative predictive value (NPV) in order to achieve the desired enrichment of the true-positive patient population. The second assay step is typically intended to rule out those patients in the enriched population that do not presently have ovarian cancer, that is, to eliminate additional true negatives from the enriched population. The second assay step may employ a higher specificity and positive predictive value (PPV), while maintaining a reasonable sensitivity, to further eliminate true negatives from the enriched population of true-positive patients. A schematic representation of the two-step screening test and the results that can be obtained with this method is provided in FIG. 1.

One of skill in the art will recognize that these assay steps may be performed as a single screening test encompassing both assay steps or as two distinct tests, wherein each test encompasses one of the assay steps. A single biomarker or panel of biomarkers may be used in each assay step to achieve the desired values for sensitivity, specificity, NPV, and PPV. Algorithms may be developed to select particular biomarkers or combinations of biomarkers to achieve the desired sensitivity, specificity, NPV, and PPV for each assay step and the combined screening test comprising both assay steps.

In addition to the two-step analysis described herein above, a screening method for identifying patients with an increased likelihood of having ovarian cancer comprises performing a “single-step” or “one-step” screening method or assay in which the expression of a plurality of biomarkers that are selectively overexpressed in ovarian cancer is assessed in a patient body sample. Overexpression of at least one biomarker(s) is indicative of an increased likelihood of the patient having ovarian cancer. In particular aspects of the invention, the single or one-step screening method comprises detecting expression of a plurality of biomarkers, including, for example, HE4, CA125, glycodelin, MMP-7, Muc-1, PAI-1, CTHRC1, inhibin A, PLAU-R, prolactin, KLK-10, KLK-6, SLPI, and alpha-1 anti-trypsin, in a body sample, wherein overexpression of at least one, particularly two, more particularly three, of the biomarkers is indicative of an increased likelihood of having ovarian cancer. In particular embodiments of the single-step screening method of the invention, expression of glycodelin, HE4, and CA125 is assessed and overexpression of at least one, two, or three of the biomarkers is indicative of an increased likelihood of having ovarian cancer. See, for example, Table 54 and Experimental Example 4. One of skill in the art will appreciate that in contrast to the two-step screening method described above, the single-step screening assay does not require performing a first assay step to detect overexpression of a particular biomarker or panel of biomarkers followed by a second assay step to assess if a second biomarker or panel of biomarkers is also overexpressed in order to identify a patient with an increased likelihood of having ovarian cancer. As such, a first assay step to enrich the population for “true-positive” patients and a second assay step to rule out those patients from the enriched population that do not actually have ovarian cancer (“false positives”) is not required in the single-step screening method described herein.

The level of expression of a particular biomarker that is sufficient to constitute “overexpression” will vary depending on the specific biomarker used. In particular embodiments of the invention, a “threshold level” of expression is established for a particular biomarker, wherein expression levels above this value are deemed overexpression. A variety of statistical and mathematical methods for establishing the threshold level of expression are known in the art. A threshold expression level for a particular biomarker may be selected, for example, based on data from Receiver Operating Characteristic (ROC) plots, as described in Examples 2 and 3, or on compilations of data from normal patient samples (i.e., a normal patient population). For example, the threshold expression level may be established at the mean expression level plus two times the standard deviation, based on analysis of samples from normal patients not afflicted with ovarian cancer. One of skill in the art will appreciate that these threshold expression levels can be varied, for example, by moving along the ROC plot for a particular biomarker, to obtain different values for sensitivity, specificity, positive predictive value (PPV), and negative predictive value (NPV), thereby affecting overall assay performance.

A patient that is identified as having an increased likelihood of having ovarian cancer in accordance with the disclosed methods may be subjected to further diagnostic testing to definitively determine if the patient has ovarian cancer. “Further diagnostic testing” includes but is not limited to pelvic examination, transvaginal ultrasound, CT scan, MRI, laparotomy, laparoscopy, and biopsy. Such diagnostic methods are well known in the art. Moreover, patients classified as having an increased likelihood of having ovarian cancer that are determined by further diagnostic testing not to currently have ovarian cancer may be closely monitored on a regular basis for the development of ovarian cancer. Monitoring of such patients may include but is not limited to periodic pelvic examination, transvaginal ultrasound, CT scan, and MRI. A physician of ordinary skill in the art will appreciate appropriate techniques for monitoring patients for the development of ovarian cancer. By identifying and monitoring patients having an increased likelihood of having ovarian cancer, the screening methods of the invention may permit the detection of ovarian cancer at an earlier stage of the disease, particularly Stage 1 or Stage 2, when prognosis and disease outcome are greatly improved.

In particular embodiments, antibodies are used to detect biomarker expression at the protein level. In other aspects of the invention, biomarker expression is detected at the nucleic acid level. Kits for practicing the screening methods of the invention, including the two-step screening method, are further provided.

By “ovarian cancer” is intended those conditions classified by post-exploratory laparotomy as premalignant pathology, malignant pathology, and cancer (FIGO Stages 1-4). Staging and classification of ovarian cancer are described in detail above. “Early-stage ovarian cancer” refers to those disease states classified as Stage 1 or Stage 2 carcinoma. Early detection of ovarian cancer significantly increases 5-year survival rates. The term “screening method” refers to strategies to identify patients that have an increased likelihood of having ovarian cancer so that such patients can be selected for more aggressive diagnostic methods to definitively determine if the patients have ovarian cancer. The “screening methods” or “diagnostic screening methods” of the invention are generally not intended to definitively diagnose a patient as having or not having ovarian cancer. Rather, such methods are intended to identify women having an increased likelihood of having ovarian cancer so that these women may be definitively diagnosed using other methods (e.g., pelvic examination, transvaginal ultrasound, CT scan, MRI, laparotomy, laparoscopy, and biopsy of tissue samples). Regimens may also be instituted for monitoring patients identified as having an increased likelihood of having ovarian cancer by the present methods but that are determined not to currently have the disease.

The screening methods of the invention may be performed on a case-by-case basis or as a periodic routine screening test for the general female population. In some embodiments, the screening methods for identifying patients with an increased likelihood of having ovarian cancer may be viewed as comparable to Pap smears for the identification of patients having an increased likelihood of having cervical cancer. As used herein, “identifying patients with an increased likelihood of having ovarian cancer” is intended methods for detecting those females that are more likely to have ovarian cancer. An “increased likelihood of having ovarian cancer” is intended to mean that patients who are determined in accordance with the present methods to exhibit overexpression of particular biomarkers are more likely to have ovarian cancer than those patients who do not.

“Diagnosing ovarian cancer” is intended to include, for example, diagnosing or detecting the presence of ovarian cancer, monitoring the progression of the disease, and identifying or detecting cells or samples that are indicative of ovarian cancer. The terms diagnosing, detecting, and identifying ovarian cancer are used interchangeably herein. Definitive diagnosis of ovarian cancer will generally comprise performing a biopsy on a tissue sample from the patient.

The methods of the present invention permit superior assessment of the likelihood of having ovarian cancer when compared with proposed screening methods currently known in the art (e.g., measurement of CA125 levels). As used herein, “specificity” refers to the level at which a method of the invention can accurately identify samples that have been confirmed as nonmalignant by exploratory laparotomy (i.e., true negatives). That is, specificity is the proportion of disease negatives that are test-negative. In a clinical study, specificity is calculated by dividing the number of true negatives by the sum of true negatives and false positives. By “sensitivity” is intended the level at which a method of the invention can accurately identify samples that have been laparotomy-confirmed as positive for ovarian cancer (i.e., true positives). Thus, sensitivity is the proportion of disease positives that are test-positive. Sensitivity is calculated in a clinical study by dividing the number of true positives by the sum of true positives and false negatives. The sensitivity of the disclosed methods for the detection of ovarian cancer is at least about 70%, particularly at least about 80%, more particularly at least about 90, 91, 92, 93, 94, 95, 96, 97, 98, 99% or more. Furthermore, the specificity of the present methods is at least about 70%, particularly at least about 80%, most particularly at least about 90, 91, 92, 93, 94, 95, 96, 97, 98, 99% or more.

The term “positive predictive value” or “PPV” refers to the percentage of patients with a positive test result in the methods of the invention who actually have ovarian cancer. PPV is calculated in a clinical study by dividing the number of true positives by the sum of true positives and false positives. PPV is highly dependent upon the prevalence rates for a particular disease or condition and will shift dramatically as a result of differences in disease prevalence. Therefore, with low-prevalence diseases, such as ovarian cancer, screening tests with a relatively low PPV still have significant clinical utility. In contrast, a disease with high prevalence rates would require a higher PPV to be clinically useful. See, for example, Skates et al. (2004) J. Clin. Oncol. 22:4059-4066; Bast et al. (2005) Int. J. Gynecol. Cancer 15:274-281; Rosen et al. (2005) Gyn. Oncol. 99:267-277; and Pepe (2004) The Statistical Evaluation of Medical Tests for Classification and Prediction (Oxford University Press), all of which are herein incorporated by reference in their entirety. The PPV for the present methods of identifying patients with an increased likelihood of having ovarian cancer is generally at least about 7, 8, 9, 10, 15, 20, 25, 30% or more. In some embodiments, the PPV of a method of the invention is at least about 10%. A PPV of at least about 10% for a diagnostic screening method is considered in the art to be of clinical utility. See Skates et al., supra. The “negative predictive value” or “NPV” of a test is the percentage of patients with a negative test result who actually do not have ovarian cancer. NPV is calculated in a clinical study by dividing the number of true negatives by the sum of true negatives and false negatives. The NPV for the present methods of identifying patients with an increased likelihood of having ovarian cancer is generally at least about 80%, particularly at least about 90%, more particularly at least about 91, 92, 93, 94, 95, 96, 97, 98, 99, 99.1, 99.2, 99.3, 99.4, 99.5, 99.6, 99.7, 99.8, 99.9% or more. In some embodiments, the NPV of a method of the invention for is at least about 99%. One of skill in the art will appreciate that the PPV and NPV values for the methods of the invention are based on a prevalence-adjusted population and are reflective of the prevalence rates of ovarian cancer within the U.S. female population.

A “biomarker” is any gene or protein whose level of expression in a tissue or cell is altered compared to that of a normal or healthy cell or tissue. Biomarkers of the invention are selective for ovarian cancer. By “selectively overexpressed in ovarian cancer” is intended that the biomarker of interest is overexpressed in ovarian cancer but is not overexpressed in conditions classified as nonmalignant, benign, and other conditions that are not considered to be clinical disease. Thus, detection of the biomarkers of the invention permits the differentiation of samples indicative of an increased likelihood of having ovarian cancer or the presence of ovarian cancer from normal samples (i.e., samples from patients that are ovarian-cancer free) and samples that are indicative of nonmalignant and benign proliferation. Biomarkers of the invention may be referred to herein interchangeably as “ovarian cancer biomarkers,” “markers,” or “ovarian cancer markers.”

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Fragments and variants of biomarker genes and proteins are also encompassed by the present invention. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

The biomarkers of the invention include any gene or protein that is selectively overexpressed in ovarian cancer, as defined herein above, and may comprise known biomarkers as well as those presently unknown in the art. In particular embodiments, biomarkers are secreted proteins or proteins that are predicted to encode membranous proteins with transmembrane segments and extracellular domains. Biomarkers of interest include HE4, CA125, glycodelin, MMP-7, Muc-1, PAI-1, CTHRC1, inhibin, PLAU-R, prolactin, KLK-10, KLK-6, and SLPI, alpha-1 anti-trypsin (AAT), Imp-2, FLJ10546, FLJ23499, MGC13057, SPON1, S100A1, SLC39A4, TACSTD2, MBG2, HETKL27 (MAL2), Cox-1, protein kinase C-iota, cadherin-6, ADPRT, matriptase, folate receptor, claudin 4, mesothelin, aquaporin 5, cofilin 1, gelsolin, clusterin, alpha tetranectin, vitronectin. pregnancy-associated plasma protein-A (PAPP-A), and folistatin. Biomarkers of particular interest include but are not limited to HE4, CA125, Glycodelin, MMP-7, Muc-1, PAI-1, CTHRC1, inhibin, PLAU-R, prolactin, KLK-10, KLK-6, SLPI, and alpha-1 anti-trypsin (AAT). Biomarkers of more particular interest include HE4, glycodelin, MMP-7, SLPI, PLAU-R, Muc-1, inhibin A, and PAI-1.

HE4 is a protein that was first observed in human epididymis tissue, and the name “HE4” is an abbreviation of “Human Epididymis Protein 4”. Subsequent studies have shown that HE4 protein is also present in the female reproductive tract and other epithelial tissues. The HE4 gene resides on human chromosome 20q12-13.1, and the 20q12 chromosome region has been found to be frequently amplified in ovarian carcinomas. Studies have shown that HE4 is expressed by ovarian carcinoma cells. The protein is N-glycosylated and is secreted extracellularly. See, for example, Drapin et al. (2005) Cancer Research 65(6): 2162-9; Hellstrom et al. (2003) Cancer Research 63: 3695-3700; and Bingle et al. (2002) Oncogene 21: 2768-2773.

CA-125 is a high molecular weight, cell surface glycoprotein detected in the serum of a large proportion of patients with ovarian epithelial cancer (OEC). However, while the percentage is high (75-90%) in advanced stages of this disease, it is only elevated in 50% of the patients with Stage 1 disease. Use of CA-125 as a marker for OEC is problematic because the molecule is also expressed in a number of normal and pathological conditions including menstruation, pregnancy, endometriosis, inflammatory diseases and other types of cancer. Improved sensitivity and specificity for OEC has been reported among post menopausal women. See, for example, Bast et al. (1998) Int'l J. Biological Markers 13:170-187; and Moss et al. (2005) J. Clin. Pathol. 58:308-312.

Glycodelin is a member of the lipocalin superfamily with several distinctive actions in cell recognition and differentiation principally in the reproductive axis. Previously, this glycoprotein has been named progesterone-associated endometrial protein (PAEP) and placental protein 14 (PP-14). The name change was in part initiated because glycodelin is not synthesized by the endometrium or placenta. Glycodelin has been purified from amniotic fluid as 28 kDa molecule in SDS gels and reported to be synthesized by the normal ovary and by malignant ovarian tumors. Its presence has been reported in serum. See generally Seppala et al. (2002) Endocrine Reviews 23:401-430; Pala et al. (1997) J. Chromatography B 704:25-34; Meerit Kamarainen et al. (1996) Amer. J Pathology 148:1435-1443.

Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. The enzyme encoded by the MMP-7 (matrilysin) gene degrades proteoglycans, fibronectin, elastin and casein and differs from most MMP family members in that it lacks a conserved C-terminal protein domain. The enzyme is involved in wound healing, and studies in mice suggest that it regulates the activity of defensins in intestinal mucosa. The MMP-7 gene is part of a cluster of MMP genes which localize to chromosome 11q22.3. MMP-7 is expressed in epithelial cells of normal and diseased tissue. It is known to be expressed in tumors of the breast, colon, and prostate, among others. It is abundant in ovarian carcinoma cells, but not detectable by IHC in normal ovarian epithelial tissue.

Muc-1 (mucin1, EMA, PEM, episialin) is a large cell surface mucin glycoprotein expressed by most glandular and ductal epithelial cells and some hematopoietic cell lineages. Functionally, Muc-1 is believed be involved with cell protection and lubrication, and may play a role in cell adhesion, and/or cell signaling. The Muc-1 gene contains seven exons and produces several different alternatively spliced variants. The major expressed form of Muc-1 uses all seven exons and is a type 1 transmembrane protein with a large extracellular tandem repeat domain. The tandem repeat domain is highly O-glycosylated and alterations in glycosylation have been shown in epithelial cancer cells. Muc-1 has been found to be elevated in many types of cancer, most notably with advanced breast cancer. Standing alone as a serum-based marker, it would have little specificity, but could be utilized as a prognostic aid, in conjunction with other, more specific markers.

PAI-1 (i.e., plasminogen activator inhibitor type 1) is a serine or cysteine proteinase inhibitor. The PAI-1 mRNA is 2876 bp in length, and the encoded protein is 402 amino acids long. The calculated molecular weight is 42,769 Da, whereas the affinity-purified protein is reported to be approximately 50,000 Da, as determined by SDS gel electrophoresis. PAI-1 plays a role in inhibiting extracellular matrix degradation by PLAU and is a putative unregulated c-Myc target gene. PAI-1 also plays a key role in controlling coagulation and tissue remodeling. PAI-1 limits the production of plasmin and serves to keep fibrinolysis in check. Some physiological functions involving the inhibition of plasmin by PAI-1 include ovulation, cell migration, and epithelial cell differentiation. High PAI-1 levels in cancer indicates poor prognosis for survival of many human cancers, including breast and lung cancers. Pappot et al. (Nov. 29, 2005) Lung Cancer [Epub ahead of print]; Chazaud et al. (2002) American Journal of Pathology 160:237-246.

Collagen triple helix repeat containing 1 (CTHRC1) was identified in a screen for differentially expressed sequences in balloon-injured versus normal arteries. In studies by Pyagay et al., CTHRC1 expression was not detectable in normal arteries. However, on injury it was transiently expressed by fibroblasts of the remodeling adventitia and by smooth muscle cells of the neointima. It was also found in the matrix of calcifying human atherosclerotic plaques. CTHRC1 is a secreted 28-kDa protein that is glycosylated and highly conserved from lower chordates to mammals. A short collagen motif with 12 Gly-X-Y repeats appears to be responsible for trimerization of the protein and this renders the molecule susceptible to cleavage by collagenase. Cthrc1 mRNA expression levels are increased in response to transforming growth factor-beta and bone morphogenetic protein-4. Cell migration assays performed with CTHRC1-overexpressing fibroblasts and smooth muscle cells demonstrate that increased CTHRC1 levels are associated with enhanced migratory ability. Furthermore, CTHRC1 overexpression caused a dramatic reduction in collagen type I mRNA and protein levels. The data of Pyagay et al. indicate that the novel molecule CTHRC1 is transiently expressed in the arterial wall in response to injury where it may contribute to vascular remodeling by limiting collagen matrix deposition and promoting cell migration. Pyagay et al. (2005) Circ. Res. 96(2):261-8.

Inhibins are protein hormones that belong to the transforming growth factor β superfamily and are heterodimers consisting of α and β subunits joined by disulfide bonds. The 2 forms of inhibins (i.e., A and B) differ in the type of β subunit (i.e., βA or βB) linked to the α subunit. In women of reproductive age, inhibins are known to be secreted by granulosa cells of the ovary and circulate in blood. Inhibins vary in concentration through the menstrual cycle and during pregnancy, and impact pituitary FSH production, gametogenesis and gestational events. In post menopausal women inhibins fall to very low levels. Recent publications indicate that inhibins are diagnostic markers for ovarian cancer. See for example Robertson et al. (2002) Mol. Cell Endocrinol. 191:97-103; El-Shalakany et al. (2004) J. Obstet. Gynaecol. Res. 30: 155-161.

Plasminogen activator urokinase-receptor (PLAU-R or UPAR) is a cell surface glycoprotein with a molecular weight of approximately 60 kDa that is attached by its carboxy-terminal end to the cell membrane by GPI linkage. PLAU-R serves as a specific receptor for the serine protease urokinase plasminogen activator (uPA) that is involved in basement membrane/extracellular matrix remodeling in both normal and pathological processes. Soluble PLAU-R, released from the cell surface, has been reported to be at elevated concentrations in serum in several types of human cancer including colorectal, breast and ovarian cancer. See for example Sier et al. (1998) Cancer Res. 58:1843-1849; and Begum et al. (2004) Anticancer Research 24:1981-1986.

Prolactin is a 198-amino acid, 23 kDa protein hormone secreted in significant quantities by the anterior pituitary gland. In concert with estrogen, prolactin plays an important role in the initiation of mammary gland growth and in lactation. In addition, prolactin is thought to have a significant role in cell growth and immune function. A recent report using a commercially available ELISA kit reported that prolactin levels in serum were significantly elevated in ovarian cancer. See generally Mor et al. (2005) Proc. Natl Acad. Sci. 102: 7677-7682.

Kallikreins are a subgroup of serine proteases having diverse physiological functions. They are clustered as a group on chromosome 19q13. Growing evidence suggests that many kallikreins are implicated in carcinogenesis and some have potential as novel cancer and other disease biomarkers.

KLK-6 is one of the fifteen kallikrein subfamily members located in a cluster on chromosome 19. The encoded enzyme is regulated by steroid hormones. In tissue culture, the enzyme has been found to generate amyloidogenic fragments from the amyloid precursor protein, suggesting a potential for involvement in Alzheimer's disease. KLK-6 has been verified as a secreted protein, and found to be elevated in ovarian cancer patients. Thus, it has been hypothesized to have value as a serological marker for this disease.

The human kallikrein 10 gene (KLK10, also known as normal epithelial cell specific 1 gene (NES-1)) is a member of the kallikrein gene family. The gene product is a secreted serine protease whose concentration in various biological fluids is known to be altered in some disease states. In particular, KLK10 is expressed in epithelial cells of the ovary and this expression has been reported to be elevated in serum of patients with ovarian cancer. Thus, KLK10 may serve as a serum biomarker for ovarian cancer. See for example Liu-Ying et al. (2003) Cancer Res. 63:807-811; Yousef and Diamandis (2003) Thromb Haemost. 90:7-16.

Secretory Leukocyte Protease Inhibitor (SLPI) is a protein that was first isolated in human parotid gland secretions. Subsequent studies have shown that the SLPI protein is also present in saliva and numerous mucosal surfaces such as those of the lung, nasal passages, cervix, and seminal vesicles.

SLPI is believed to act as a defense against chronic lung ailments because it is a potent inhibitor of neutrophil elastase, whose presence is heightened in chronic inflammatory lung diseases and which can destroy many of the components of lung tissue. SLPI also inhibits the release of histamine from mast cells, so it is also active in the allergic response. Protection of fetal membranes and cervical tissue is also thought to be a function of SLPI.

The gene which encodes the SLPI protein has been found to be up-regulated in ovarian cancer, and a significant difference has been found between the elevated levels of SLPI in the serum of patients with malignant ovarian cancer as opposed to the levels found in patients with benign ovarian cysts or normal patients. The SLPI protein has a mass of 12 kDa, is non-glycosylated, hydrophobic, and cationic. See generally Hollander et al. (2003) Cancer Cell International 3:14; Helmig et al. (1995) Eur. J. Obstet. Gynecol. Reprod. Biol. 59(1):95-101; Hough et al. (2001) Cancer Research 61: 3869-3876; and Tsukishiro et al. (2005) Gynecologic Oncology 96: 516-519.

Alpha-1 anti-trypsin (AAT) is an acute phase protein synthesized by the liver and the principal serum inhibitor of proteolytic enzymes such as trypsin, chymotrypsin, plasmin and thrombin. In an inflammatory reaction the serum concentration of AAT may be elevated as much as 3-4 fold. The molecule exists as a number of genetic variants with a monomer molecular weight of 40-50 kDa. The mean normal serum concentration (mg/ml) has been reported to be 2.21±0.35 and 2.14±0.37. Interest in AAT as an ovarian cancer biomarker resulted from an in-house 2D gel electrophoresis/mass spec. study that indicated that AAT was elevated in the serum of ovarian cancer patients. See for example Song et al. (1994) J. Affect. Disorders 30:283-288; Ledue et al. (1993) Clin. Chim. Acta. 223: 73-28.

Although the above biomarkers have been discussed in detail, any biomarker whose overexpression is selective for ovarian cancer can be used to practice the invention, including biomarkers known in the art and those not yet identified. Such biomarkers include genes and proteins that are, for example, involved in defects in DNA replication/cell cycle control, cell growth and proliferation, escape from apoptosis, angiogenesis or lymphogenesis, or the mechanisms of cancer cell motility and invasion.

Of particular interest are biomarkers that are selectively overexpressed in early-stage ovarian cancer. By “selectively overexpressed in early-stage ovarian cancer” is intended that the biomarker of interest is overexpressed in stage 1 or stage 2 ovarian cancer states but is not overexpressed in normal samples or in conditions classified as nonmalignant, benign, and other conditions that are not considered to be clinical disease. One of skill in the art will appreciate that early-stage ovarian cancer biomarkers include those genes and proteins specific for ovarian cancer that are initially overexpressed in stage 1 or stage 2 and whose overexpression persists throughout the advanced stages of the disease, as well as biomarkers that are only overexpressed in stage 1 or stage 2 ovarian cancer. Detection of expression of biomarkers that are selectively overexpressed in early-stage ovarian cancer may permit the earlier detection and diagnosis of ovarian cancer and, accordingly, improve patient prognosis.

The methods of the invention comprise detecting the expression of a plurality of biomarkers. As used herein, a “plurality” of biomarkers refers to 2, 3, 4, 5, 6, 7, 8, 9, 10 or more biomarkers. In particular, when the two-step screening method of the invention is used to identify patients having an increased likelihood of having ovarian cancer, a plurality of biomarkers may refer to the detection of at least one biomarker during the first assay step and at least one additional biomarker during the second assay step. One of skill in the art will also recognize that a panel of biomarkers can be used to identify patients with an increased likelihood of having ovarian cancer in accordance with the present methods. A panel of biomarkers may comprise any number or combination of biomarkers of interest. In some embodiments, a panel comprising a plurality of biomarkers selected from the group consisting of HE4, CA125, Glycodelin, MMP-7, Muc-1, PAI-1, CTHRC1, inhibin, PLAU-R, prolactin, KLK-10, KLK-6, and SLPI, alpha-1 anti-trypsin (AAT). In other embodiments, a panel of biomarkers selected from the subset of biomarkers comprising CA125, HE4, glycodelin, MMP-7, SLPI, PLAU-R, Muc-1, inhibin A, and PAI-1 is provided.

Any female patient or patient population may be assessed using the screening and diagnostic methods of the invention. For example, the methods disclosed herein may be performed on the general female patient population, as well as on the narrower population of post-menopausal women. The term “post-menopausal” is understood by those of skill in the art. In particular embodiments, post-menopausal generally refers to, for example, women over the age of 55. In particular embodiments, the screening methods are performed routinely (e.g., annually, every two years, etc.) on the general female population. Regular screening of patients may begin, for example, at the onset of menses, at age 30, or at the beginning of menopause. Screening of the high-risk patient population, as defined herein below, will typically be performed on a routine basis independent of patient age. Patients who are both asymptomatic and symptomatic (i.e., displaying characteristic symptoms of ovarian cancer, such as pelvic or abdominal pain or swelling) can be assessed for an increased likelihood of having ovarian using the screening and diagnostic methods of the invention. Women that are at a low-risk of developing ovarian and those that are considered high-risk based on clinical and family history risk factors may also be assessed using the present methods. Patients considered “high-risk” based on such clinical and family history risk factors include but are not limited to patients living with breast cancer, colon cancer, or breast/ovarian syndrome, women with a first-degree relative with ovarian cancer (e.g., mother, daughter, or sister), patients positive for at least one breast cancer gene (BRCA 1 or 2), and women suffering from HNPCC (i.e., Hereditary non-polyposis colorectal cancer).

In one aspect of the invention, the target population for the screening and diagnostic methods is the group of asymptomatic patients classified as high-risk on the basis of, for example, the above-referenced clinical and familial risk factors. This high-risk, asymptomatic population represents more than 8 million women in the U.S. It is recognized that the methods, compositions, and kits of the invention will be of particular utility to patients having an enhanced risk of developing ovarian cancers and to their physicians. Patients recognized in the art as having an increased risk of developing ovarian cancers include, for example, patients having a familial history of ovarian cancer and patients of advancing age (i.e., typically women over 55 years of age).

A number of clinical conditions or characteristics not directly related to ovarian cancer may exist in the patient populations tested in accordance with the methods of the invention, thereby interfering with the results of the screening and diagnostic methods disclosed herein. Such clinical conditions and characteristics are referred to herein as “interfering substances and pathologies” and include but are not limited to pregnancy (first trimester), breast cancer, chronic hepatitis, colon cancer, oral contraceptive therapy, coronary artery disease, deep vein thrombosis, diabetes, endometriosis, hormone replacement therapy, menstruation, multiple myeloma, ovarian cysts/polycystic disease, polymyalgia, polymyositis, rectal cancer, rheumatoid arthritis, systemic lupus rythematosus (SLE), and warfarin treatment. Additional exemplary interfering pathologies include uterine conditions (e.g., myomas, adenomyosis, and endometrial cancer), ovarian conditions (e.g., benign growths such as functional cysts, theca-lutein cysts, pregnancy luteoma, sclerocystic ovaries, serous cystadenoma, mucinous cystadenoma, cystic teratoma, fibroma, thecoma, and Brenner tumor; neoplasms; and malignant conditions such as cystadenocarcinoma and adenocarcinoma), fallopian tube conditions (e.g., tubo-ovarian abscess, hydrosaplinx, parovarian cyst, ectopic pregnancy, and cancer of the fallopian tubes), bowel conditions (e.g., distention with gas and/or feces, diverticulitis, ileitis, appendicitis, and colon cancer), and other miscellaneous conditions (e.g., distended bladder, pelvic kidney, urachal cyst, abdominal wall hematoma, abdominal wall abscess, and retroperitoneal neoplasms, such as lymphoma, sarcoma, and teratoma. In particular embodiments, the biomarkers, threshold expression levels, and mathematical models used in the screening and diagnostic methods described herein will be selected so as to minimize the effects of interfering substances and pathologies on the performance (i.e., the specificity, sensitivity, PPV, and NPV) of the claimed methods.

By “body sample” is intended any sampling of cells, tissues, or bodily fluids from a patient in which expression of a biomarker can be detected. Examples of such body samples include but are not limited to blood (e.g., whole blood, blood serum, blood having platelets removed, etc.), lymph, ascitic fluids, urine, gynecological fluids (e.g., ovarian, fallopian, and uterine secretion, menses, etc.), biopsies, and fluids obtained during laparoscopy. Body samples may be obtained from a patient by a variety of techniques including, for example, by venipuncture, by scraping or swabbing an area, or by using a needle to aspirate bodily fluids or tissues. Methods for collecting various body samples are well known in the art. In particular embodiments, the body sample comprises blood or serum. The present inventors have recognized that the methods for the collection and storage of blood samples, more particularly serum samples, affect the performance of the methods disclosed herein. Several studies indicate that body sample, particularly serum sample, collection and storage methods are critical to achieve acceptable assay performance. See, for example, Diamandis (2004) J. Natl. Cancer Institute 95:353-356 and Thavasu et al. (1992) J. Immunol. Meth. 153:115-124. which is herein incorporated reference in its entirety. An exemplary method for serum collection and storage is provided in Example 1.

Any methods available in the art for the detection biomarker expression can be used to practice the invention. The expression of a biomarker of the invention can be detected on a nucleic acid level or a protein level. In order to determine overexpression, the body sample to be examined may be compared with a corresponding body sample that originates from a healthy person. That is, the “normal” level of expression is the level of expression of the biomarker in a body sample from a patient that is not afflicted with ovarian cancer. Such a sample can be present in standardized form. In some embodiments, determination of biomarker overexpression requires no comparison between the body sample and a corresponding body sample that originates from a normal person.

Any biomarker or combination of biomarkers of the invention, as well as any known ovarian cancer biomarkers, may be used in the methods, compositions, and kits of the present invention. In general, it is preferable to use biomarkers for which the difference between the level of expression of the biomarker in a body sample from a patient afflicted with ovarian cancer and the level of expression of the same biomarker in a “normal” body sample (i.e., from an ovarian cancer free patient) is as great as possible. Although this difference can be as small as the limit of detection of the method for assessing expression of the biomarker, it is preferred that the difference be at least greater than the standard error of the assessment method, and optimally a difference of at least 2, 3, 4, 5, 6, 7, 8, 9, 10, 15, 20, 25-fold or greater than the level of expression of the same biomarker in a normal body sample. The “normal” level of expression of a biomarker of the invention may be determined by assessing expression of the biomarker in a body sample obtained from a non-ovarian cancer afflicted patient. Alternatively, and particularly as further information becomes available as a result of the routine performance of the methods described herein, average values for biomarker expression levels may be used.

As described above, in some aspects of the invention, a threshold level of expression of a particular biomarker, above which the biomarker is considered to be overexpressed in a patient sample, may be established. Methods for selecting the threshold value include but are not limited to analysis of the ROC plot for the biomarker or analysis of the biomarker expression level for a normal patient population. Exemplary threshold or “cutoff” values include (1) the mean expression level plus two times the standard deviation, as determined from a population of normal patient samples and (2) expression levels selected from the ROC curve that represent the highest value of sensitivity plus specificity. The threshold level of expression for a particular biomarker may be determined relative to the expression level in a normal patient population. Persons of skill in the art will appreciate that other methods for selecting appropriate threshold expression values can be used to practice the invention.

Methods for detecting biomarkers of the invention comprise any methods that determine the quantity or the presence of the biomarkers either at the nucleic acid or protein level. Such methods are well known in the art and include but are not limited to western blots, northern blots, southern blots, ELISA, immunoprecipitation, immunofluorescence, flow cytometry, immunocytochemistry, multiplex bead-based immunoassays, nucleic acid hybridization techniques, nucleic acid reverse transcription methods, and nucleic acid amplification methods. In particular embodiments, overexpression of a biomarker is detected on a protein level using, for example, antibodies that are directed against specific biomarker proteins. These antibodies can be used in various methods such as Western blot, ELISA, multiplex bead-based immunoassay, immunoprecipitation, or immunocytochemistry techniques. The multiplex bead-based assays used to practice the present invention include but are not limited to the Luminex technology described in U.S. Pat. Nos. 6,599,331, 6,592,822, and 6,268,222, all of which are herein incorporated by reference in their entirety. In particular embodiments, the Luminex LabMAP® system is utilized, as described in International Publication No. WO 2005/016126, which is herein incorporated by reference it its entirety.

In some embodiments of the invention, antibodies specific for biomarker proteins are utilized to detect the expression of a plurality of biomarker proteins in a body sample in order to identify patients with an increased likelihood of having ovarian cancer. The method comprises obtaining a body sample from a patient, particularly a serum sample, contacting the body sample with antibodies directed to a plurality of biomarkers that are selectively overexpressed in ovarian cancer, and detecting antibody binding to determine if the biomarkers are overexpressed in the patient sample. In the screening methods of the invention, overexpression of the biomarkers is indicative of an increased likelihood of having ovarian cancer. Patients classified by the present screening methods as having an increased likelihood of having ovarian cancer are subjected to further diagnostic testing to detect the presence of ovarian cancer. Female patients identified by the present screening methods for ovarian cancer that are determined to not be currently suffering from ovarian cancer are regularly monitored in order to potentially detect ovarian cancer at an earlier stage.

As described above, in particular aspects of the invention, the screening methods for identifying patients with an increased likelihood of having ovarian cancer may comprise a two-step analysis. That is, the method comprises performing a first assay step comprising detecting the expression of a first biomarker or a first panel of biomarkers in a body sample and determining if the first biomarker or panel of biomarkers is overexpressed. If a positive result is obtained in the first assay step (i.e., the first biomarker or panel of biomarkers is overexpressed in the body sample), the method further comprises performing a second assay step comprising detecting the expression of a second biomarker or a second panel of biomarkers and determining if the second biomarker or panel of biomarkers is overexpressed. Overexpression of both the first and second biomarkers or panels of biomarkers (i.e., a positive result in both assay steps) is indicative of an increased likelihood of having ovarian cancer. In particular embodiments, detection of expression of the biomarkers in the first and second assay steps is performed at the protein level and comprises contacting the body sample, more particularly a serum sample, with antibodies specific for the particular biomarkers of interest and detecting antibody binding. In certain methods, biomarker protein expression is detected using an ELISA or multiplex bead-based immunoassay format.

In the “two-step” screening methods of the invention, the biomarker or panel of biomarkers used in the first step may be employed to maximize sensitivity and NPV. That is, the first screening step may be designed to maximize the number of true negatives classified as negative by the methods of the invention, thereby eliminating a significant percentage of true negatives from further analysis. Therefore, the patient population classified as positive by the first assay step will be enriched in true positive patients. The second screening step in the two-step methods may be designed to maximize PPV and specificity, while maintaining a reasonable sensitivity, in order to identify women with the highest likelihood of having ovarian cancer. Specificity, sensitivity, PPV, and NPV values may be determined for each individual assay step in the method, as described above. When a two-step screening method is used, combined sensitivity, specificity, PPV, and NPV values may determined for the method as a whole. In particular embodiments, the two-step method for identifying patients having an increased likelihood of having ovarian cancer will have a combined sensitivity of at least 90%, a combined specificity of at least 98%, a combined PPV of at least 10%, and a combined NPV of at least 99.9%. One of skill in the art will further appreciate that while two-step screening methods have been described in detail, similar screening methods comprising 3, 4, 5, or more assay steps are also encompassed by the present invention. Such follow-on assay steps may rule out other diseases, such as cardiovascular conditions, from the presence of ovarian cancer.

In some embodiments of the invention, algorithms or mathematical models may be applied to develop “test rules” for determining when a patient sample is “positive” (i.e., indicative of an increased likelihood of having ovarian cancer). For example, when expression of HE4, CA125, PLAU-R, glycodelin, Muc-1, and PAI-1 is detected, the screening test is considered positive if (1) any three of HE4, CA125, glycodelin, PAI-1, and PLAU-R are positive (i.e., overexpressed) or if (2) HE4 and CA125 are both overexpressed; otherwise, the test is considered negative. In a further example, the test is considered positive if (1) any three of HE4, CA125, glycodelin, MMP-7, and PLAU-R are positive (i.e., overexpressed) or if (2) HE4 is overexpressed and also any one of CA125, glycodelin, MMP-7, or PLAU-R is positive; otherwise, the test is deemed negative. One of skill in the art will appreciate that a variety of such test rules can be developed and applied in the present methods for identifying patients with an increased likelihood of having ovarian cancer such as logistical regression. Other mathematical models that can be applied are described in Zhou et al. (2002) Statistical Methods in Diagnostic Medicine (Wiley, New York), which is herein incorporated by reference in ts entirety.

In other aspects of the invention, methods for diagnosing ovarian cancer in a patient comprise obtaining a body sample from a patient, contacting the body sample with antibodies directed to a plurality of biomarkers that are selectively overexpressed in ovarian cancer, and detecting antibody binding to determine if the biomarkers are overexpressed in the patient sample. In the diagnostic methods of the invention, overexpression of the biomarkers is indicative of the presence of ovarian cancer.

In still other embodiments of the invention, methods for assessing the efficacy of a therapy for treating ovarian cancer in a patient are provided. Such methods typically comprise comparing the level of expression of a plurality of biomarkers of the invention in a first patient body sample procured prior to the initiation of therapy with that from a second sample obtained following administration of at least a portion of the therapy. A significantly lower level of expression of the biomarkers in the second patient sample relative to that of the first sample obtained prior to the initiation of the therapy is a positive indication of the efficacy of the therapy for treating ovarian cancer in the patient, whereas a significantly higher level of expression of the biomarkers in the second sample is a negative indication of the efficacy of the therapy. As used herein, a “positive indication of the efficacy of the therapy” means that the therapy is producing beneficial results in the treatment of ovarian cancer (e.g., tumor regression, etc.). A “negative indication of the efficacy of the therapy” is intended to mean that the therapy is not having beneficial effects with respect to treatment of ovarian cancer. A negative indication of the efficacy of the particular treatment may be related to, for example, the dosage. At higher dosages, the therapy may be efficacious.

One of skill in the art will recognize that in these methods the term “therapy” includes any therapy for treating ovarian cancer, including but not limited to chemotherapy, radiation therapy, surgical removal of tumor tissue, gene therapy, and biologic therapy. The methods of the invention may be used to evaluate a patient before, during, and after therapy to evaluate, for example, a reduction in tumor burden.

The invention additionally provides a monitoring method for assessing the regression or progression of ovarian cancer in a patient comprising detecting in a first patient sample at a first time point the level of expression of a plurality of biomarkers of the invention, repeating this analysis with a second patient sample obtained at a later time point, and comparing the level of expression of the biomarkers at the two time points. A significantly higher level of expression of the biomarkers in the patient body sample at the later time point indicates that the ovarian cancer has progressed, whereas a significantly lower level of expression is an indication that the ovarian cancer has regressed. As used herein, “regression of ovarian cancer” is intended to mean that the condition of the patient with respect to ovarian cancer has improved, as characterized by, for example, decreased tumor size. “Progression of ovarian cancer” means that the condition of the patient with respect to ovarian cancer has worsened, as characterized by, for example, increased tumor size, metastasis, etc. The meanings of the terms “regression” and “progression” with respect to disease states will be understood by those of skill in the art.

In certain aspects of the invention, biomarker expression is detected at the protein level using antibodies. The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab′, F′(ab)₂, Fv, single chain antibodies, diabodies), and recombinant peptides comprising the foregoing.

The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen-binding or variable region of the intact antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and is still capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Polyclonal antibodies can be prepared by immunizing a suitable subject (e.g., rabbit, goat, mouse, or other mammal) with a biomarker protein immunogen. The antibody titer in the immunized subject can be monitored over time by standard techniques, such as with an enzyme linked immunosorbent assay (ELISA) using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:550-52; Kenneth (1980) in Monoclonal Antibodies: A New Dimension In Biological Analyses (Plenum Publishing Corp., NY); and Lerner (1981) Yale J. Biol. Med., 54:387-402).

Alternative to preparing monoclonal antibody-secreting hybridomas, a monoclonal antibody can be identified and isolated by screening a recombinant combinatorial immunoglobulin library (e.g., an antibody phage display library) with a biomarker protein to thereby isolate immunoglobulin library members that bind the biomarker protein. Kits for generating and screening phage display libraries are commercially available (e.g., the Pharmacia Recombinant Phage Antibody System, Catalog No. 27-9400-01; and the Stratagene SurfZAP Θ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

The compositions of the invention further comprise monoclonal antibodies and variants and fragments thereof that specifically bind to biomarker proteins of interest. The monoclonal antibodies may be labeled with a detectable substance as described below to facilitate biomarker protein detection in the sample. Such antibodies find use in practicing the methods of the invention. Monoclonal antibodies having the binding characteristics of the antibodies disclosed herein are also encompassed by the present invention. Compositions further comprise antigen-binding variants and fragments of the monoclonal antibodies, hybridoma cell lines producing these antibodies, and isolated nucleic acid molecules encoding the amino acid sequences of these monoclonal antibodies.

Antibodies having the binding characteristics of a monoclonal antibody of the invention are also provided. “Binding characteristics” or “binding specificity” when used in reference to an antibody means that the antibody recognizes the same or similar antigenic epitope as a comparison antibody. Examples of such antibodies include, for example, an antibody that competes with a monoclonal antibody of the invention in a competitive binding assay. One of skill in the art could determine whether an antibody competitively interferes with another antibody using standard methods.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”; these epitopes are not arranged sequentially), or both linear and nonlinear amino acid residues. Typically epitopes are short amino acid sequences, e.g. about five amino acids in length. Systematic techniques for identifying epitopes are known in the art and are described, for example, in U.S. Pat. No. 4,708,871. Briefly, a set of overlapping oligopeptides derived from the antigen may be synthesized and bound to a solid phase array of pins, with a unique oligopeptide on each pin. The array of pins may comprise a 96-well microtiter plate, permitting one to assay all 96 oligopeptides simultaneously, e.g., for binding to a biomarker-specific monoclonal antibody. Alternatively, phage display peptide library kits (New England BioLabs) are currently commercially available for epitope mapping. Using these methods, the binding affinity for every possible subset of consecutive amino acids may be determined in order to identify the epitope that a given antibody binds. Epitopes may also be identified by inference when epitope length peptide sequences are used to immunize animals from which antibodies are obtained. Epitopes may also be defined by carbohydrate side chains present as either N-linked or O-linked oligosaccharides present on glycoproteins.

Antigen-binding fragments and variants of the monoclonal antibodies disclosed herein are further provided. Such variants will retain the desired binding properties of the parent antibody. Methods for making antibody fragments and variants are generally available in the art. For example, amino acid sequence variants of a monoclonal antibody described herein, can be prepared by mutations in the cloned DNA sequence encoding the antibody of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] , Val [symbol] Ile [symbol] Leu, Asp [symbol] Glu, Lys [symbol] Arg, Asn [symbol] Gln, and Phe [symbol] Trp [symbol] Tyr.

In constructing variants of the antibody polypeptide of interest, modifications are made such that variants continue to possess the desired activity, i.e., similar binding affinity to the biomarker. Obviously, any mutations made in the DNA encoding the variant polypeptide must not place the sequence out of reading frame and preferably will not create complementary regions that could produce secondary mRNA structure. See EP Patent Application Publication No. 75,444.

Preferably, variants of a reference biomarker antibody have amino acid sequences that have at least 70% or 75% sequence identity, preferably at least 80% or 85% sequence identity, more preferably at least 90%, 91%, 92%, 93%, 94% or 95% sequence identity to the amino acid sequence for the reference antibody molecule, or to a shorter portion of the reference antibody molecule. More preferably, the molecules share at least 96%, 97%, 98% or 99% sequence identity. For purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from the reference antibody by as few as 1 to 15 amino acid residues, as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

One of skill in the art will recognize that optimization of reagents and conditions, for example, antibody titer and parameters for detection of antigen-antibody binding, is needed to maximize the signal to noise ratio for a particular antibody. Antibody concentrations that maximize specific binding to the biomarkers of the invention and minimize non-specific binding (or “background”) will be determined. In particular embodiments, appropriate antibody titers are determined by initially testing various antibody dilutions on patient serum samples. The design of assays to optimize antibody titer and detection conditions is standard and well within the routine capabilities of those of ordinary skill in the art. Some antibodies require additional optimization to reduce background and/or to increase specificity and sensitivity.

Furthermore, one of skill in the art will recognize that the concentration of a particular antibody used to practice the methods of the invention will vary depending on such factors as time for binding, level of specificity of the antibody for the biomarker protein, and method of body sample preparation. Moreover, when multiple antibodies are used in a single sample, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

The antibodies used to practice the invention are selected to have high specificity for the biomarker proteins of interest. Methods for making antibodies and for selecting appropriate antibodies are known in the art. See, for example, Celis, ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., serum samples) in mind and for binding specificity.

In other embodiments, the expression of a biomarker of interest is detected at the nucleic acid level. Nucleic acid-based techniques for assessing expression are well known in the art and include, for example, determining the level of biomarker mRNA in a body sample. Many expression detection methods use isolated RNA. Any RNA isolation technique that does not select against the isolation of mRNA can be utilized for the purification of RNA from serum samples (see, e.g., Ausubel et al., ed., (1987-1999) Current Protocols in Molecular Biology (John Wiley & Sons, New York). Additionally, large numbers of blood, serum, or tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically utilize pairs of oligonucleotide primers that are specific for the biomarker of interest. Methods for designing oligonucleotide primers specific for a known sequence are well known in the art.

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

In one embodiment of the invention, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

Kits for practicing the screening and diagnostic methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and methods for its use.

In particular embodiments, kits for use in screening methods for identifying patients with an increased likelihood of having ovarian cancer comprise a plurality of antibodies directed to biomarker proteins of interest, particularly wherein the biomarkers are selected from the group consisting of HE4, CA125, glycodelin, MMP-7, Muc-1, PAI-1, CTHRC1, inhibin A, PLAU-R, prolactin, KLK-10, KLK-6, SLPI, and alpha-1 anti-trypsin. Chemicals for the detection of antibody binding to the biomarker may also be included in the kit. Other reagents for detecting expression of biomarkers using antibodies in an ELISA or multiplex bead-based immunoassay format may be further included in a kit of the invention. In other embodiments, kits for use in the screening methods disclosed herein comprise nucleic acid probes for the detection of expression of a plurality of biomarkers of interest in a body sample, particularly wherein the biomarkers are selected from the group consisting of HE4, CA125, glycodelin, MMP-7, Muc-1, PAI-1, CTHRC1, inhibin A, PLAU-R, prolactin, KLK-10, KLK-6, SLPI, and alpha-1 anti-trypsin.

In certain aspects of the invention, kits for performing the two-step screening method for identifying patients having an increased likelihood of having ovarian cancer are provided. Kits may be designed to perform the two-step screening method as two separate tests. Such kits include reagents for performing a single assay step (i.e., the first or second assay step). In other embodiments, the kits comprise reagents so that the two-step method may be performed as a single test (i.e., include reagents for both the first and second assay steps). Further kits encompassed by the invention find use in practicing the single-step screening method described herein above. The kits may further comprise descriptions of algorithms or mathematical models to be applied with the one-step or two-step screening methods, as well as automated platforms that implement the algorithms/mathematical models with little or no human intervention. The kits may be further provided such that each step in the two-step method is performed in an automated, semi-automated, or manual fashion.

Kits for diagnosing ovarian cancer are also provided. Such kits may include antibodies or nucleic acid probes that are specific for detection of a plurality of biomarkers that are selectively overexpressed in ovarian cancer, more particularly HE4, CA125, glycodelin, MMP-7, Muc-1, PAI-1, CTHRC1, inhibin A, PLAU-R, prolactin, KLK-10, KLK-6, SLPI, and alpha-1 anti-trypsin.

One of skill in the art will further appreciate that any or all steps in the screening and diagnostic methods of the invention could be implemented by personnel or, alternatively, performed in an automated fashion. That is, the methods can be performed in an automated, semi-automated, or manual fashion. Furthermore, the methods disclosed herein can also be combined with other methods known or later developed to permit a more accurate identification of patients having an increased likelihood of having ovarian cancer or a more reliable diagnosis of ovarian cancer.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation:

### EXPERIMENTAL

### Example 1

### Exemplary Method for Collection of Blood, Processing of Blood into Serum Serum Storage Conditions, and Serum Shipping Conditions

### Blood Collection

A sufficient quantity of blood was drawn by venipuncture into a Becton Dickinson Vacutainer red-topped collection tubes containing no additive (catalog number 366430). The lot number of the blood collection tube was recorded. Whole blood was not refrigerated or frozen. Processing of the whole blood to serum began immediately after collection of the blood.

### Serum Preparation Procedure

Blood collection tubes were placed upright in a rack and stored at room temperature. The blood was allowed to clot for a minimum of 60 minutes and a maximum of 90 minutes from time of collection. Blood was thoroughly clotted before centrifugation. The time required for complete clotting for each sample was recorded.

After blood was thoroughly clotted, the samples were centrifuged at 1300×g for 10 minutes using either a swing-head or fixed-angle centrifuge rotor. Tubes were carefully removed from the centrifuge to avoid disturbing the red blood cells on the bottom.

The stopper was removed from each tube without disturbing the cell pellet. The serum from each donor was combined into a labeled tube using a disposable transfer pipette without disturbing the cell layer or allowing any cells into the pipette. If cells were disturbed during transfer, the samples were re-centrifuged as described above.

Serum samples from each donor were mixed by gently inverting the tube five times. One mL aliquots were placed in labeled tubes and frozen at −20° C. to −80° C. Serum was not stored at room temperature for more than two hours or at 2-8° C. for more than eight hours prior to freezing.

### Example 2

### Ovarian Biomarker Selection Study—Individual Biomarker Analysis

The purpose of the biomarker selection study was to evaluate the initial clinical utility of a set of fourteen candidate ovarian cancer biomarkers. The premise of the study was to be able to identify and quantitate the amount of each biomarker in a patient's serum. Biomarker protein expression in serum samples was detected using commercial or novel biomarker-specific antibodies in an ELISA format. Fourteen target biomarkers were analyzed, as described below in Table 1:

TABLE 1
Candidate markers

| | |
|---|---|
| HE4 | CA125 |
| SLPI | Glycodelin |
| KLK6 | MMP7 |
| KLK10 | PLAU-R |
| CTHRC1 | Inhibin |
| PAI-1 | Prolactin |
| MUC1 | Alpha-1 anti-trypsin |

### Target Population and Demographics of the Study

In evaluations of this type, it is essential that the samples tested are representative of the target clinical population and that normal controls are demographically matched. A total of 900 patient serum samples were analyzed in this study. 200 of the samples were from ovarian cancer patients in various stages of the disease (i.e., the 200 samples were evenly split among Stage 1, 2, 3 ovarian cancer samples). A total of 500 normal sera were used in the study. 104 of the normal serum samples were from post-menopausal women (set as women over the age of 55 for the present study) to establish the baseline levels of each biomarker and to calculate the appropriate threshold cut-off values. The remaining 396 normal serum samples were evenly split between pre-menopausal women under the age of 55 and post-menopausal women over the age of 55. This distribution permitted the investigation of any age or hormone-related patient distributions. The remaining 200 samples in the study were collected from donors with various conditions/diseases, as described herein above (i.e., “interfering conditions” or “interfering pathologies”). The inclusion of these samples allowed for the analysis of any interfering pathologies that may affect individual biomarker performance. Summaries of the patient populations analyzed and study demographics are provided in Tables 2-4.

TABLE 2
Demographics of sample cases in the study

| | | | | |
|---|---|---|---|---|
| | | | Interfering | Threshold |
| Characteristics | Ovarian Cancer | Study Normals | Pathologies | Normals |

| Total number of samples | 200 | | 396 | | 200 | | 104 | |
|---|---|---|---|---|---|---|---|---|
| Age at diagnosis (years) | | | | | | | | |
| Mean (std) | 55.5 | (11.5) | 55.3 | (9.7) | 57.0 | (18.3) | 63.0 | (6.9) |
| Range | 19-81 | | 40-84 | | 20-97 | | 41-80 | |
| Age group distribution | | | | | | | | |
| <=55 | 100 | (50%) | 201 | (50.8%) | 92 | (46%) | 2 | (1.9%) |
| >55 | 100 | (50%) | 195 | (49.2%) | 108 | (54%) | 102 | (98.1%) |
| Race, n | | | | | | | | |
| African-American | 10 | (5%) | 3 | (1%) | 12 | (6%) | 0 | |
| Caucasian | 183 | (92%) | 389 | (99%) | 185 | (92.5%) | 98 | (100%) |

| | | | | | | | |
|---|---|---|---|---|---|---|---|
| Other | 6 | (3%) | 1 | (<1%) | 3 | (1.5%) | 0 |

| | | | | |
|---|---|---|---|---|
| Missing | 1 | 3 | 0 | 6 |

TABLE 3
Clinical Characteristics of Ovarian Cancer Patients in Study

| | Frequency | Percent |
|---|---|---|
| Ovarian Cancer Stage, n | 200 | |
| 1 | 67 | 33.5% |
| 2 | 66 | 33% |
| 3 | 67 | 33.5% |
| Histological Classification, n | 197 | |
| Clear Cell | 10 | 5% |
| Endometrioid | 39 | 19% |
| Serous | 23 | 12% |
| Mucinous | 36 | 18% |
| Adenocarcinoma | 49 | 25% |
| Papillary | 25 | 13% |
| Unclassified | 13 | 7% |
| Stromal Sarcoma | 2 | 1% |

TABLE 4
Distribution of Interfering Substances/Pathologies

| | Category | Number | Percent |
|---|---|---|---|
| Hormonal | 1st trimester pregnancy | 10 | 5.0 |
| | Contraceptive Pills | 5 | 2.5 |
| | Hormone Replacement Therapy | 9 | 4.5 |
| | Menstruation | 9 | 4.5 |
| Cancer | Breast Cancer | 27 | 13.5 |
| | Colon Cancer | 15 | 7.5 |
| | Rectal Cancer | 2 | 1.0 |
| | Multiple Myeloma | 1 | 0.5 |
| Vascular | Coronary Artery Disease | 9 | 4.5 |
| | Deep Vein Thrombosis | 1 | 0.5 |
| | Warfarin Treatment | 15 | 7.5 |
| Metabolic | Diabetes | 10 | 5.0 |
| | Chronic Hepatitis | 10 | 5.0 |
| Gyn | Endometriosis | 25 | 12.5 |
| | Ovarian cysts/polycystic | 10 | 5.0 |
| Inflammatory | Polymyalgia | 10 | 5.0 |
| | Polymyositis | 3 | 1.5 |
| | Rheumatoid Arthritis | 27 | 13.5 |
| | SLE | 2 | 1.0 |

### General Description of Automation and Validation Analysis for the Study

All 900 sera samples were processed in duplicate for each of the 14 markers. An automated assay system with barcode tracking was utilized in generating the marker selection data. The assays run in this marker selection study were all completed using a Tecan Evo automated robotic liquid handler. The use of this platform allowed all 900 samples to be processed for each marker in a single experimental run (one run per day). The precision of the automated platform was verified before any study samples were processed.

All of the ELISA assays were processed for this study using a buffered solution as the diluent for the standard curve. This was done to standardize, as much as possible, the protocols for each of the assays.

The normal sera used in this study were obtained from community blood collection centers compliant with local IRB customs. While these sera can be considered ‘normal’ it must be noted that these donors were not screened by a medical professional to confirm their disease free or normal status

### Detection of Expression of HE4 in Serum Samples (A Representative Example)

### Materials and Methods

A. Coating of Assay Plates:

ELISA 96-well plates were coated with 100 μl/well of the primary antibody, anti-HE4 monoclonal 90.1 #6, at 2 μg/ml in PBS, then the plates were incubated at 4° C. overnight. The next day, the plates were washed once with PBS, then 250 μl/well of PBS-3% BSA was added to all wells, and the plates were incubated for 2 hours at 30° C. The plates were then emptied and dried in a vacuum oven for 2 hours at room temperature. Then they were heat-sealed inside a mylar foil bag along with a desiccant pack and stored at 4° C. until used in an assay.

B. Assay Methods

The foil bags containing the assay plates were warmed to room temperature immediately prior to use in the assay. The serum samples were diluted 1:4 into PBS-1% bovine Serum-0.05% Tween 20-1 mg/ml mouse IgG. The HE4 antigen protein was diluted to 100 ng/ml, then serially diluted two-fold into PBS-1% Bovine Serum-0.05% Tween 20-1 mg/ml Mouse IgG, then all the individual standard curve samples were further diluted 1:4 into the buffer so that they would be diluted the same as the serum samples.

The diluted sera samples and standard curve samples were added to the anti-HE4 coated assay plates in 100 μl/well volumes. Then the plates were incubated for 2 hours at 30° C. The plates were next washed 5× with 250 μl/well of PBS-0.05% Tween 20. The secondary antibody, anti-HE4 monoclonal 71.1#1.13-HRP, was diluted 1:16,000 into PBS-1% bovine IgG-0.05% Tween 20-1 mg/ml mouse IgG. The secondary antibody solution was then added to the aspirated plates in 100 μl/well volumes. The plates were incubated for 1 hour at 30° C.

The plates were washed 5× with 250 μl/well of PBS-0.05% Tween 20. The developing solution, TMB(3,3′,5,5′-Tetramethylbenzidine) was warmed to room temperature prior to use, and then the TMB was added to the aspirated plates in 100 μl/well volumes.

The plates were incubated for 10 minutes at room temperature and then the Stop solution, 2N H2SO4, was added to the plates (containing the TMB) in 100 μl/well volumes.

The plates were incubated for 10 minutes at room temperature, and then they were read on the Molecular Devices SpectraMax plate reader at 450 nm, with a reference wavelength of 650 nm, using SoftMax Pro software.

The data was saved as SoftMax Pro files, and also exported as Text files for use with MS Excel.

Controls: Serum used as the High Control: Uniglobe # 72372

Serum used as the Low Control: Uniglobe # 72404

Buffer Control used was PBS-1% Bovine Serum-0.05% Tween 20-1 mg/ml Mouse IgG

The CV's for the controls across all 25 plates are summarized in Table 5.

TABLE 5
The CV's for the Controls Across All 25 Plates
Standards point-per-point CV % across
all 25 plates

| ng/ml | Mean OD | SD | CV % |
|---|---|---|---|
| 100 | 2.614 | 0.145 | 5.6 |
| 50 | 1.681 | 0.101 | 6.0 |
| 25 | 0.931 | 0.075 | 8.1 |
| 12.5 | 0.510 | 0.043 | 8.4 |
| 6.25 | 0.263 | 0.027 | 10.4 |
| 3.13 | 0.139 | 0.014 | 10.0 |
| 1.56 | 0.076 | 0.008 | 10.5 |
| 0.78 | 0.044 | 0.012 | 27.6 |

### Results (Standard Curves)

A standard curve for HE4 was prepared by diluting the HE4 protein in PBS/1% bovine serum/0.05% Tween 20-1 mg/mL of mouse IgG. Preparation of such standard curves is well known in the art. Standard curves and the levels or concentrations of the biomarkers in all serum samples were produced for the remaining biomarkers, essentially as described above for HE4, with variations that could be appreciated and implemented by one of skill in the art. Standard curve ranges and curve fit equations for the biomarkers analyzed (i.e., HE4, glycodelin, SLPI, PLAU-R, MUC-1, PAI-1, MMP-7, inhibin, CA125, CTHRC1, KLK-6, KLK-10, alpha-1 anti-trypsin, and prolactin) are presented below:

### HE4

Standard Curve experimental range=0.78 ng/ml to 100 ng/ml, in serial two-fold dilutions
Curve fit=third order polynomial, y=−1E-07x³−0.0001x²+0.0402x+0.0157; R²=0.9999
The standard curve samples and the serum samples were then both diluted 1:4 to run in the assay; since both the serum samples and the standard curve samples were diluted in the same manner there was no need to use a dilution factor during data analysis to determine the concentrations of the unknown serum samples.

### Glycodelin

Standard Curve experimental range=3 ng/ml to 100 ng/ml, in serial two-fold dilutions
Curve fit=quadratic, y=−0.0003x²+0.0478x+0.0952, R²=0.9851
The serum samples were run in the assay undiluted, so no dilution factor was used in the data analysis.

### SLPI

Standard Curve experimental range 2.38 ng/ml to 305 ng/ml, in serial two-fold dilutions
Curve fit=third order polynomial, y=7E-08x³−7E-05x²+0.0237x−0.039; R²=0.9997
The serum samples were diluted 1:6.1 in an early step of the automated sample preparation technique. Then those diluted serum samples were further diluted 1:6.6 in another automated step in order to achieve a total dilution of 1:40. Those 1:40 diluted samples were then run in the assay.
The standard curve samples were prepared at the concentrations stated above (experimental range), then those standard curve samples were all further diluted 1:6.1, and those 1:6.1 diluted samples were then run in the assay.
Since there was a discrepancy between the dilution of the serum samples (1:40) as compared to the dilution of the standard curve samples (1:6.1), all the serum sample concentration values obtained from the standard curve were multiplied by a dilution factor of “6.6” to correct for the discrepancy (1:40=1:6.1×1:6.6).
   uPar (PLAU-R)
Standard Curve experimental range=62.5 pg/ml to 4000 pg/ml, in serial two-fold dilutions
Curve fit=linear, y=0.0006x+0.0522; R²=0.9977
The serum samples were diluted 1:6 in an early step of the automated sample preparation technique. Then those diluted serum samples were further diluted 1:3 in another automated step in order to achieve a total dilution of 1:18. Those 1:18 diluted samples were then run in the assay.
The standard curve samples were prepared at the concentrations stated above (experimental range), then those standard curve samples were all further diluted 1:3, and those 1:3 diluted samples were then run in the assay.
Since there was a discrepancy between the dilution of the serum samples (1:18) as compared to the dilution of the standard curve samples (1:3), all the serum sample concentration values obtained from the standard curve were multiplied by a dilution factor of “6” to correct for the discrepancy (1:18=1:3×1:6).

### MUC-1

Standard Curve experimental range=0.3 U/ml to 600 U/ml, in serial three-fold dilutions
Curve fit=third order polynomial, y=3E-07x³-0.0001x²+0.0236x+0.0371; R²=1.0
The standard curve samples and the serum samples were then both diluted 1:2 to run in the assay; since both the serum samples and the standard curve samples were diluted in the same manner there was no need to use a dilution factor during data analysis to determine the concentrations of the unknown serum samples.

### PAI-1

Standard Curve experimental range=0.78 ng/ml to 100 ng/ml, in serial two-fold dilutions
Curve fit=linear, y=0.0139x+0.0151; R²=0.9996
The standard curve samples and the serum samples were then both diluted 1:4 to run in the assay; since both the serum samples and the standard curve samples were diluted in the same manner there was no need to use a dilution factor during data analysis to determine the concentrations of the unknown serum samples.

### MMP-7

Standard Curve experimental range=0.16 ng/ml to 20 ng/ml, in serial two-fold dilutions
Curve fit=third order polynomial, y=0.0001x³-0.0046x²+0.1578x+0.0936; R²=0.9999
The standard curve samples and the serum samples were then both diluted 1:2 to run in the assay; since both the serum samples and the standard curve samples were diluted in the same manner there was no need to use a dilution factor during data analysis to determine the concentrations of the unknown serum samples.

### Inhibin

Standard Curve experimental range=10 pg/ml to 1000 pg/ml, in serial two-fold dilutions
Curve fit=linear, y=0.0007x+0.0136; R²=0.9997
The standard curve samples and the serum samples were then both diluted 1:3 to run in the assay; since both the serum samples and the standard curve samples were diluted in the same manner there was no need to use a dilution factor during data analysis to determine the concentrations of the unknown serum samples.

### CA125

Standard Curve experimental range=15 U/ml to 400 U/ml, in serial two-fold dilutions
Curve fit=linear, y=0.0021x+0.0892; R² 0.9986
The serum samples were run in the assay undiluted, so no dilution factor was used in the data analysis.

### CTHRC1

Standard Curve experimental range=0.78 ng/ml to 100 ng/ml, in serial two-fold dilutions
Curve fit=linear, y=0.0261x+0.042; R²=0.9991
The standard curve samples and the serum samples were then both diluted 1:2 to run in the assay; since both the serum samples and the standard curve samples were diluted in the same manner there was no need to use a dilution factor during data analysis to determine the concentrations of the unknown serum samples.

### KLK-6

Standard Curve experimental range=0.78 ng/ml to 100 ng/ml, in serial two-fold dilutions
Curve fit=third order polynomial, y=−4E-07x³+0.0001x²+0.0133x+0.0662; R²=1.0
The standard curve samples and the serum samples were then both diluted 1:2 to run in the assay; since both the serum samples and the standard curve samples were diluted in the same manner there was no need to use a dilution factor during data analysis to determine the concentrations of the unknown serum samples.

### KLK-10

Standard Curve experimental range=1 ng/ml to 80 ng/ml, in serial two-fold dilutions
Curve fit=linear, y=0.0758x−0.0312; R²=0.9976
The standard curve samples and the serum samples were then both diluted 1:4 to run in the assay; since both the serum samples and the standard curve samples were diluted in the same manner there was no need to use a dilution factor during data analysis to determine the concentrations of the unknown serum samples.

### Alpha 1 Anti-Trypsin

Standard Curve experimental range=3.3 ng/ml to 90 ng/ml, in serial two-fold dilutions
Curve fit=quadratic, y=−0.0002x²+0.0412x+0.0592; R²=0.9997
The serum samples were diluted 1:250,000, and then those diluted samples were run in the assay. The standard curve samples were serially diluted as stated above (experimental range), and then those samples were run in the assay.
Since there was a discrepancy between the dilution of the serum samples (1:250,000) as compared to the dilution of the standard curve samples (undiluted beyond the serial dilutions), all the serum sample concentration values obtained from the standard curve were multiplied by a dilution factor of “250,000” to correct for the discrepancy.

### Prolactin

Standard Curve experimental range=2 ng/ml to 240 ng/ml, in serial two-fold dilutions
Curve fit=linear, y=0.0049x+0.0363; R²=0.9917
The standard curve samples and the serum samples were then both diluted 1:5 to run in the assay; since both the serum samples and the standard curve samples were diluted in the same manner there was no need to use a dilution factor during data analysis to determine the concentrations of the unknown serum samples.

### Results—Individual Marker Performance

Among the 500 normal cases used in the present study, 104 normal cases were used as control group to select the threshold cutoff point. Table 6 provides the mean, standard deviation, range, mean+2*std, mean+3*std based on these 104 threshold normal cases for each marker. Table 7 and Table 8 provide the mean standard deviation, range, mean+2*std in all normal study cases (396 cases) and with normal study cases from patients over the age of 55 (195 or less). The mean and standard deviations were very similar between the normal threshold control cases and normal study cases for patients over the age of 55 for each biomarker except for markers HE4 and Inhibin. See Tables 6 and 8.

TABLE 6
Mean and STD of Each Biomarker in 104 Normal Control Cases

| Variable | N | Mean | Std Dev | Min | Max | Mean + 2 * std | Mean + 3 * std | Best Cutoff |
|---|---|---|---|---|---|---|---|---|
| HE4 | 104 | 1.7 | 0.91 | 0.5 | 6.6 | 3.5 | 4.4 | 2.2 |
| CA125 | 104 | 6.9 | 6.42 | 0.6 | 42.6 | 19.7 | 26.1 | 20 |
| GLY | 104 | 1.8 | 2.75 | 0.0 | 18.6 | 7.4 | 10.1 | 5 |
| MMP | 104 | 3.4 | 1.05 | 1.9 | 7.0 | 5.5 | 6.5 | 4 |
| Muc1 | 104 | 12.5 | 10.71 | 0.1 | 51.8 | 33.9 | 44.6 | 12 |
| PAI | 104 | 74.8 | 24.92 | 16.4 | 143.2 | 124.7 | | 95 |
| CTRHC1 | 104 | 3.2 | 1.16 | 1.2 | 6.2 | 5.5 | | 4 |
| INH | 104 | 0.6 | 2.80 | 0.0 | 26.9 | 6.2 | 9.0 | 2 |
| Plau-R | 104 | 1945.4 | 534.03 | 1056.00 | 4248.0 | 3013.4 | 3547.5 | 2399.9 |
| PROLAC | 104 | 10.2 | 10.94 | 3.1 | 102.2 | 32.0 | 43.0 | 11 |
| KLK10 | 104 | 0.8 | 0.51 | 0.3 | 2.3 | 1.8 | | 0.9 |
| KLK6 | 104 | 2.2 | 2.39 | 0.3 | 16.3 | 7.0 | 9.4 | 5 |
| SLPI | 104 | 65.7 | 14.38 | 40.2 | 134.5 | 94.5 | 108.9 | 65 |

TABLE 7
Mean and STD of Each Biomarker in 396 Normal Study Cases

| Variable | N | Mean | Std Dev | Min | Max | Mean + 2 * std |
|---|---|---|---|---|---|---|
| HE4 | 396 | 1.6 | 2.1 | 0.2 | 36.4 | 5.8* |
| CA125 | 396 | 10.4 | 14.2 | 1.7 | 212.4 | 38.7* |
| GLY | 396 | 5.0 | 10.2 | 0.0 | 98.4 | 25.3* |
| MMP | 396 | 3.0 | 1.1 | 0.8 | 10.4 | 5.1 |
| Muc1 | 396 | 8.3 | 8.2 | 0.0 | 52.1 | 24.7 |
| PAI | 396 | 73.2 | 28.0 | 18.8 | 145.7 | 129.2 |
| CTRHC1 | 396 | 2.9 | 1.2 | 0.4 | 8.7 | 5.2 |
| INH | 360 | 11.3 | 28.1 | 0.0 | 265.4 | 67.5* |
| PLAUR | 396 | 2086.0 | 782.0 | 822.0 | 6384.00 | 3650 |
| PROLAC | 396 | 11.6 | 9.5 | 2.0 | 138.0 | 30.7 |
| KLK10 | 396 | 0.9 | 0.4 | 0.2 | 2.5 | 1.7 |
| KLK6 | 396 | 1.7 | 1.7 | 0.1 | 18.0 | 5.1 |
| SLPI | 396 | 59.6 | 18.0 | 31.3 | 305.0 | 95.6 |

*The values in normal cases were very different from the values in normal control cases, which means these markers might be sensitive due to age effect.

TABLE 8
Mean and STD of Each Biomarker in Normal Study Cases
(Patients Over the Age of 55)

| | | | Std | | | |
|---|---|---|---|---|---|---|
| Variable | N | Mean | Dev | Min | Max | Mean + 2 * std |
| HE4 | 195 | 2.0 | 2.8 | 0.2 | 36.4 | 7.6* |
| CA125 | 194 | 10.6 | 5.6 | 2.3 | 35.7 | 21.9 |
| GLY | 195 | 1.7 | 2.3 | 0.0 | 24.8 | 6.3 |
| MMP | 195 | 3.2 | 1.2 | 0.8 | 10.4 | 5.6 |
| Muc1 | 195 | 8.9 | 8.4 | 0.0 | 36.6 | 25.7 |
| PAI | 195 | 75.6 | 27.9 | 19.1 | 145.7 | 131.3 |
| CTRHC1 | 195 | 3.2 | 1.1 | 1.2 | 7.1 | 5.4 |
| INH | 177 | 5.2 | 26.6 | 0.0 | 265.4 | 58.5** |
| PLAUR | 195 | 2209.5 | 752.1 | 1122.0 | 5100.0 | 3713.7 |
| PROLAC | 195 | 10.8 | 11.1 | 2.0 | 138.0 | 32.9 |
| KLK10 | 195 | 0.9 | 0.4 | 0.2 | 2.4 | 1.7 |
| KLK6 | 195 | 1.8 | 2.0 | 0.1 | 18.0 | 5.7 |
| SLPI | 195 | 60.3 | 14.2 | 31.3 | 126.9 | 88.6 |

*The standard deviation was larger in the normal study cases than in the normal control cases for patients over the age of 55
**The mean and standard deviation were larger in the normal study cases than in the normal control cases for patients over the age of 55

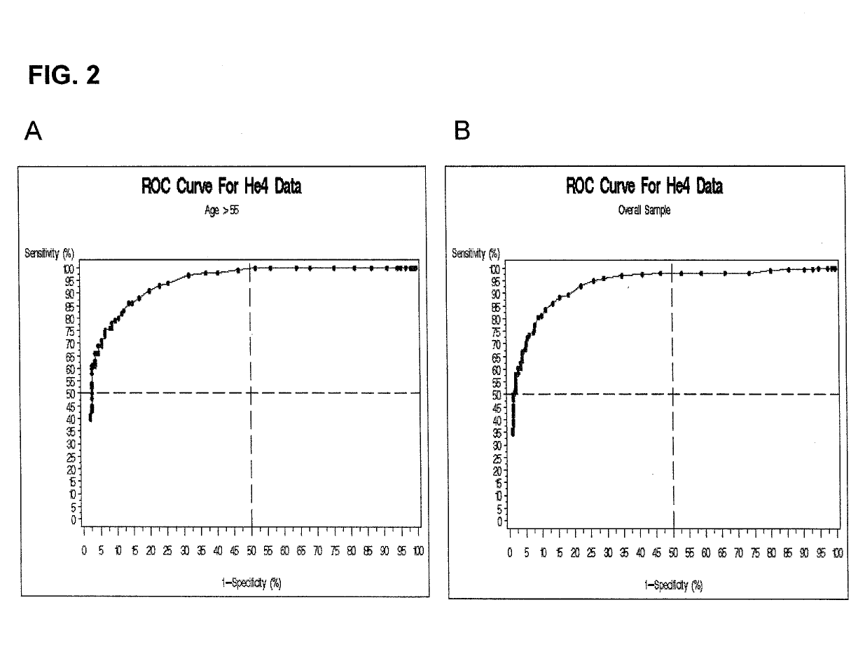

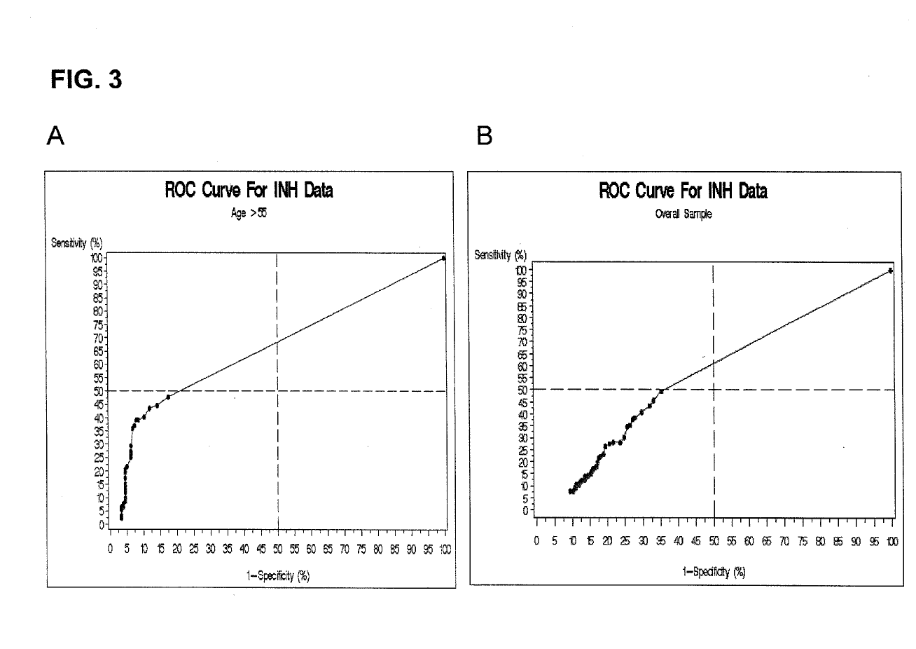

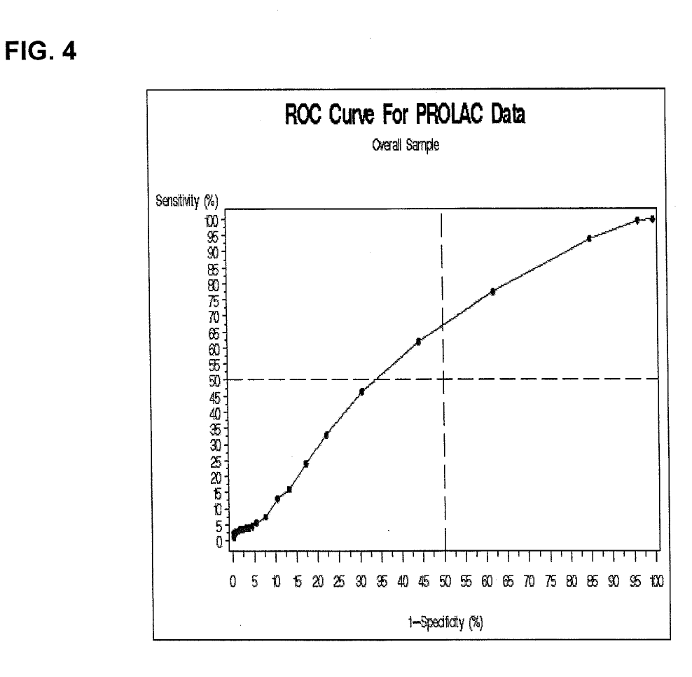

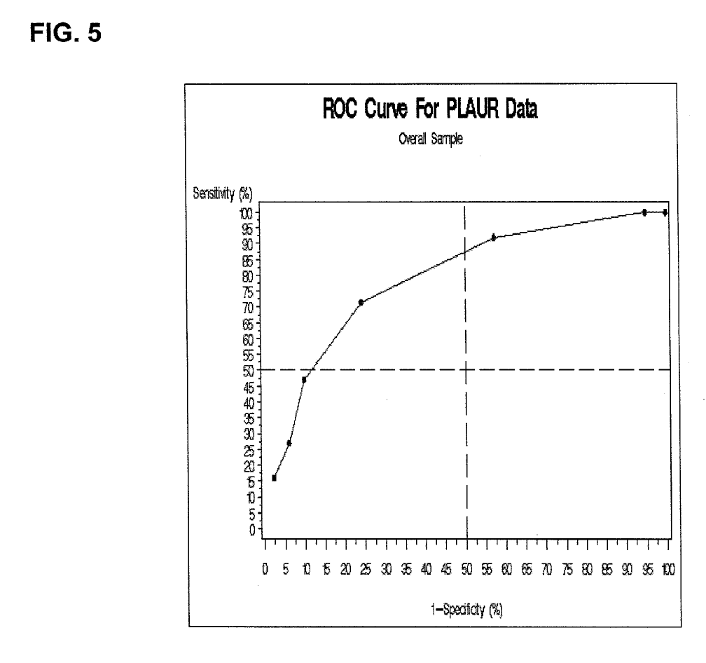

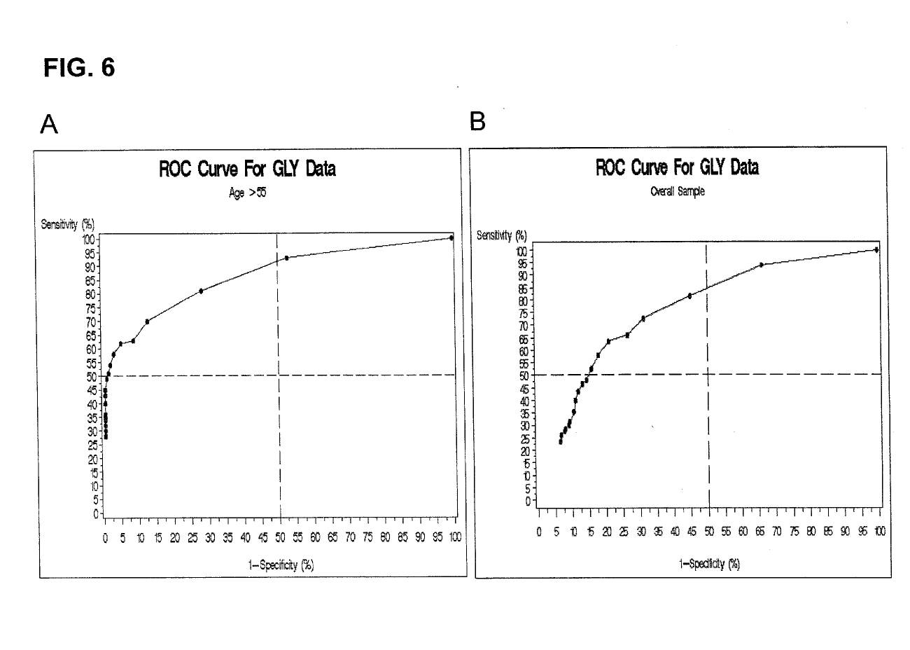

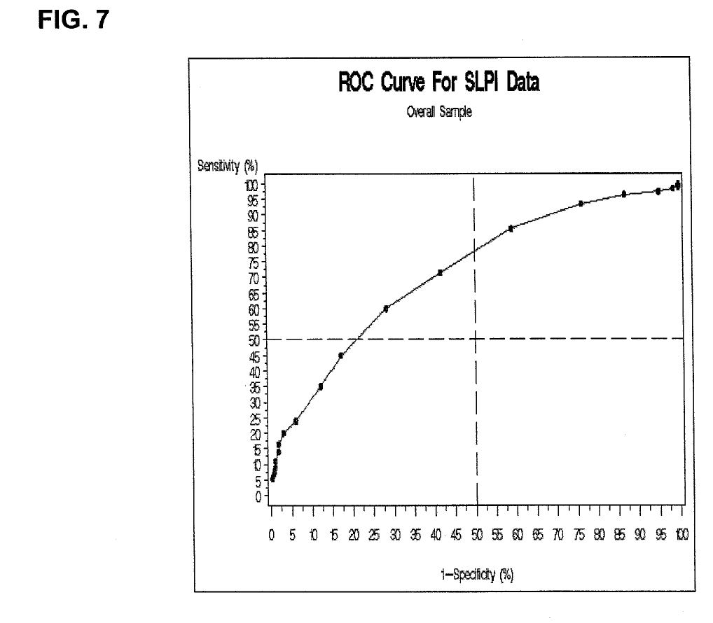

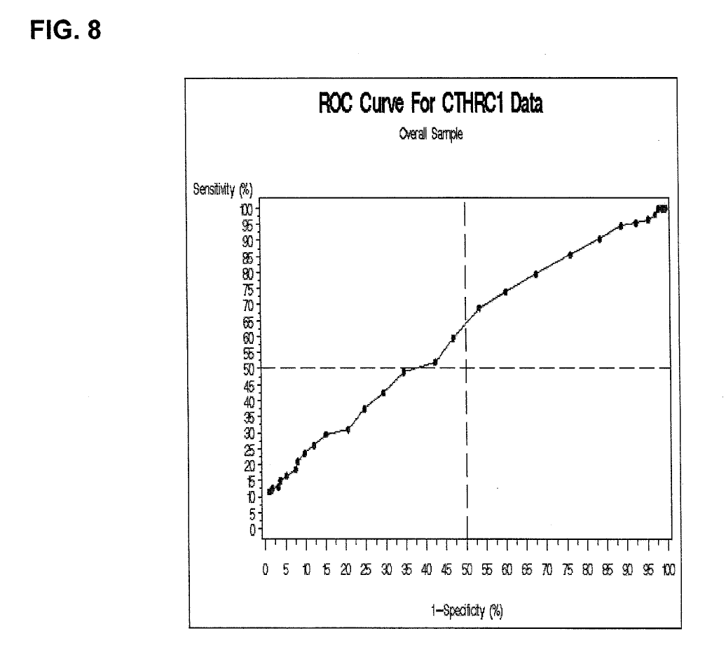

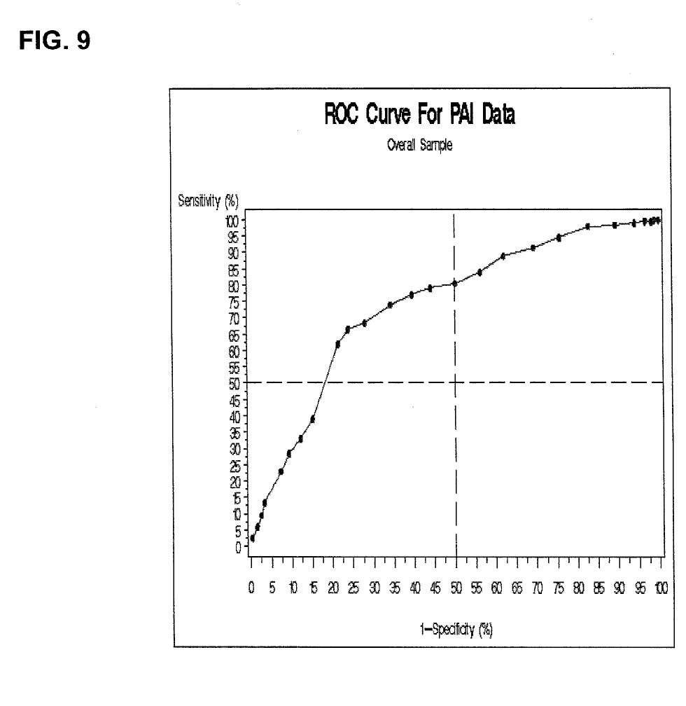

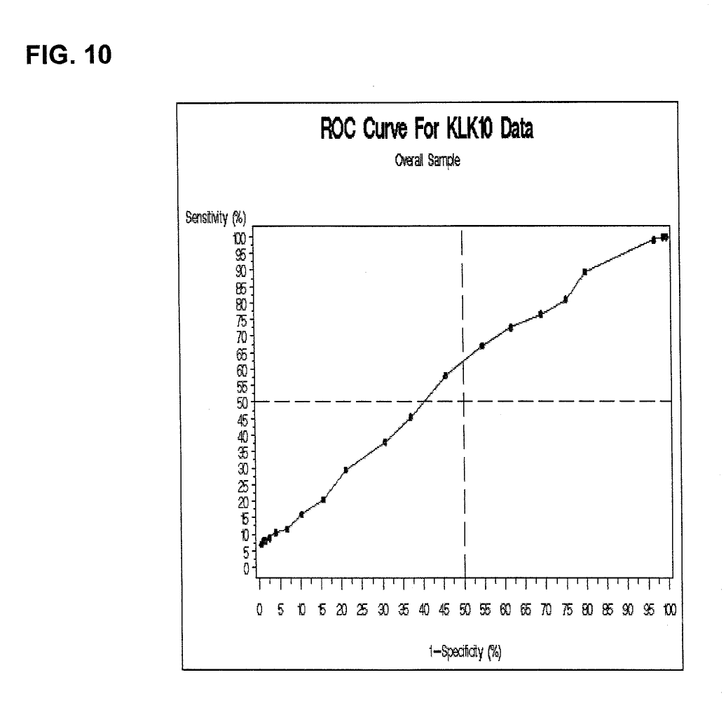

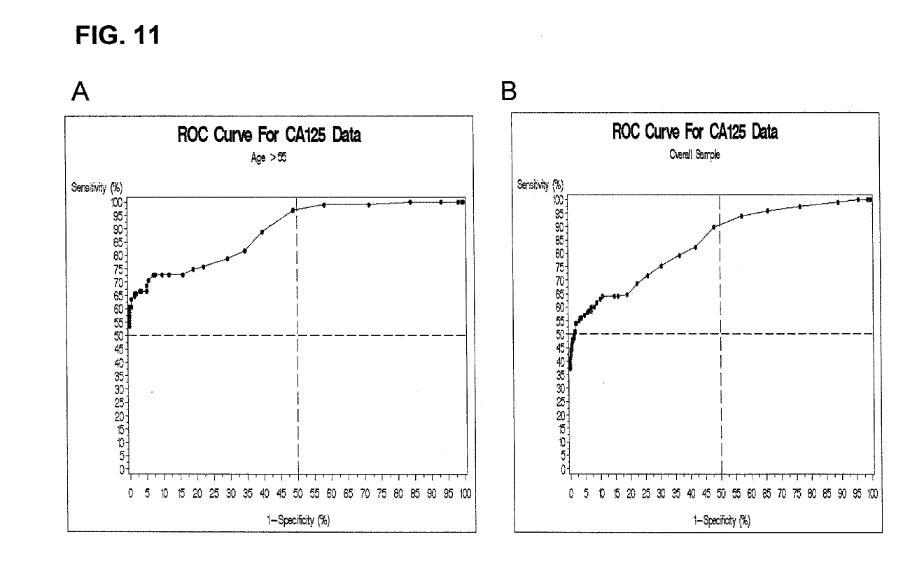

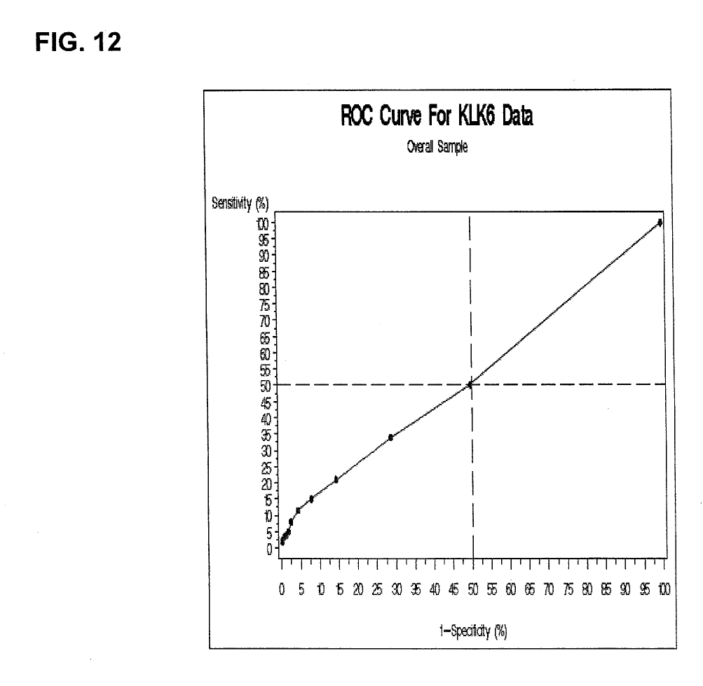

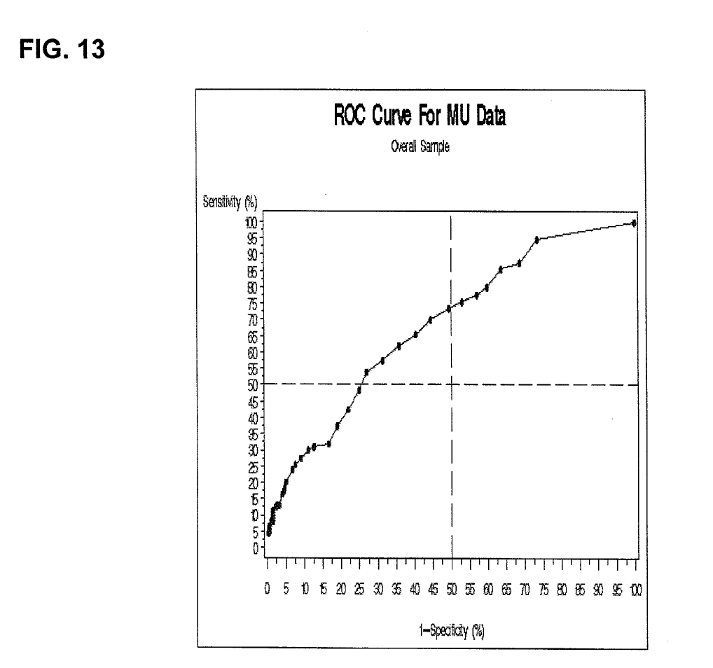

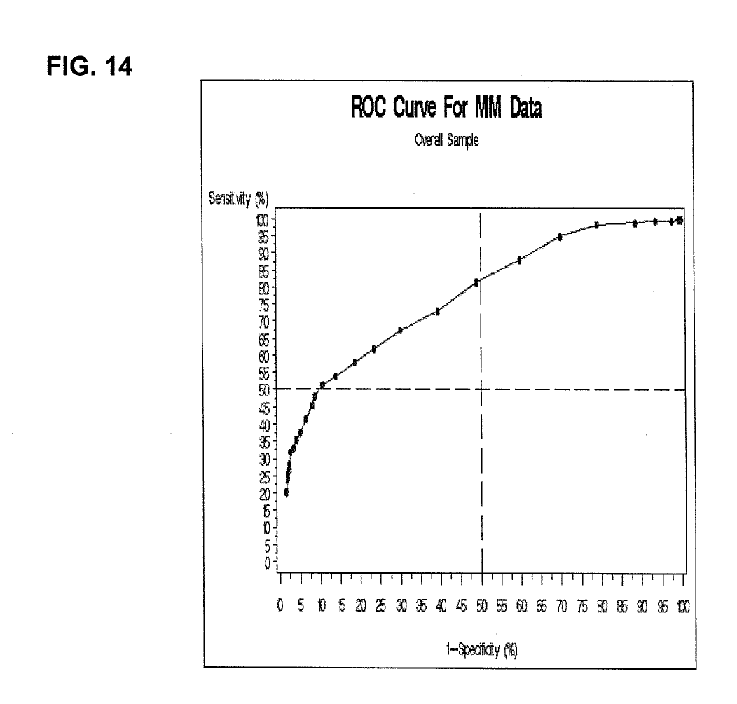

For each individual marker, the ROC curve was obtained. The ROC curve only consists of the 396 normal cases and the 200 ovarian cancer cases. The 104 normal cases as control and 200 interfering substance/pathology cases were not included in this analysis. The ROC curves for HE4, inhibin A, prolactin, PLAU-R, glycodelin, SLPI, CTHRC1, PAI-1, KLK-10, CA125, KLK-6, MUC-1, and MMP-7 are presented in FIGS. 2-14, respectively.

Also, the percentage of positive and negative samples were determined for each marker by age group, by cancer stage, and by interfering substance/pathology category. These determinations were based on two kinds of cutoff points: (1) mean+2*std, and (2) the best cutoff from the ROC curve. The best cutoff points were selected from the ROC curve with the highest value of sensitivity plus specificity. The best cutoff point was listed in Table 6. Summaries of the positive results for each biomarker by age group, cancer stage, and interfering substance/pathology category are presented in tables 9 to 47.

### Biomarker: HE4

TABLE 9
Summary by Age Group for HE4

| | | Best Cutoff from | | | | | | | |
|---|---|---|---|---|---|---|---|---|---|
| | | ROC plot | | | | Mean + 2 * std | | | |
| | | Age <= 55 | | Age > 55 | | Age <= 55 | | Age > 55 | |
| Group | Result | N | % | N | % | N | % | N | % |
| IP | Positive | 57 | 63.33 | 98 | 90.74 | 41 | 45.56 | 81 | 75.00 |
| | Negative | 33 | 36.67 | 10 | 9.26 | 49 | 54.44 | 27 | 25.00 |
| | Total | 90 | 100.00 | 108 | 100.00 | 90 | 100.00 | 108 | 100.00 |
| NORMAL | Positive | 15 | 7.46 | 39 | 20.00 | 3 | 1.49 | 13 | 6.67 |
| | Negative | 186 | 92.54 | 156 | 80.00 | 198 | 98.51 | 182 | 93.33 |
| | Total | 201 | 100.00 | 195 | 100.00 | 201 | 100.00 | 195 | 100.00 |
| OvCA | Positive | 81 | 81.00 | 91 | 91.00 | 58 | 58.00 | 75 | 75.00 |
| | Negative | 19 | 19.00 | 9 | 9.00 | 42 | 42.00 | 25 | 25.00 |
| | Total | 100 | 100.00 | 100 | 100.00 | 100 | 100.00 | 100 | 100.00 |

TABLE 10
Summary by Interfering Substance/Pathology Category for HE4

| | | Best Cutoff | | | |
|---|---|---|---|---|---|
| | | from ROC | | | |
| | | plot | | Mean + 2 * std | |
| Group | Result | N | % | N | % |
| Tumors | Positive | 38 | 84.44 | 27 | 60.00 |
| | Negative | 7 | 15.56 | 18 | 40.00 |
| | Total | 45 | 100.00 | 45 | 100.00 |
| Metabolic Disorder | Positive | 20 | 100.00 | 20 | 100.00 |
| | Negative | 0 | 0.00 | 0 | 0.00 |
| | Total | 20 | 100.00 | 20 | 100.00 |
| Hormonal Fluctuation | Positive | 23 | 69.70 | 16 | 48.48 |
| | Negative | 10 | 30.30 | 17 | 51.52 |
| | Total | 33 | 100.00 | 33 | 100.00 |
| Gynecologic Disorder | Positive | 17 | 48.57 | 12 | 34.29 |
| | Negative | 18 | 51.43 | 23 | 65.71 |
| | Total | 35 | 100.00 | 35 | 100.00 |
| Vascular Disorder | Positive | 22 | 88.00 | 19 | 76.00 |
| | Negative | 3 | 12.00 | 6 | 24.00 |
| | Total | 25 | 100.00 | 25 | 100.00 |
| Chronic Immuno. Disorder | Positive | 37 | 88.10 | 29 | 69.05 |
| | Negative | 5 | 11.90 | 13 | 30.95 |
| | Total | 42 | 100.00 | 42 | 100.00 |

TABLE 11
Summary by Cancer Stage for HE4

| | |
|---|---|
| Best Cutoff | |
| from ROC plot | Mean + 2 * std |

| Stage | Result | N | % | N | % |
|---|---|---|---|---|---|
| 1 | Positive | 57 | 85.07 | 42 | 62.69 |
| | Negative | 10 | 14.93 | 25 | 37.31 |
| | Total | 67 | 100.00 | 67 | 100.00 |
| 2 | Positive | 53 | 80.30 | 41 | 62.12 |
| | Negative | 13 | 19.70 | 25 | 37.88 |
| | Total | 66 | 100.00 | 66 | 100.00 |
| 3 | Positive | 62 | 92.54 | 50 | 74.63 |
| | Negative | 5 | 7.46 | 17 | 25.37 |

### Biomarker Muc-1

TABLE 12
Summary by Age Group for Muc-1

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 38 | 42.22 | 53 | 49.07 | 4 | 4.44 | 9 | 8.33 |
| | Negative | 52 | 57.78 | 55 | 50.93 | 86 | 95.56 | 99 | 91.67 |
| | Total | 90 | 100.00 | 108 | 100.00 | 90 | 100.00 | 108 | 100.00 |
| NORMAL | Positive | 49 | 24.38 | 58 | 29.74 | 2 | 1.00 | 4 | 2.05 |
| | Negative | 152 | 75.62 | 137 | 70.26 | 199 | 99.00 | 191 | 97.95 |
| | Total | 201 | 100.00 | 195 | 100.00 | 201 | 100.00 | 195 | 100.00 |
| OvCA | Positive | 56 | 56.00 | 52 | 52.00 | 4 | 4.00 | 12 | 12.00 |
| | Negative | 44 | 44.00 | 48 | 48.00 | 96 | 96.00 | 88 | 88.00 |
| | Total | 100 | 100.00 | 100 | 100.00 | 100 | 100.00 | 100 | 100.00 |

TABLE 13
Summary by Interfering Substance/Pathology Category for Muc-1

| | | Best Cutoff | | | |
|---|---|---|---|---|---|
| | | from ROC plot | | Mean + 2 * std | |
| Group | Result | N | % | N | % |
| Tumors | Positive | 20 | 44.44 | 3 | 6.67 |
| | Negative | 25 | 55.56 | 42 | 93.33 |
| | Total | 45 | 100.00 | 45 | 100.00 |
| Metabolic Disorder | Positive | 8 | 40.00 | 1 | 5.00 |
| | Negative | 12 | 60.00 | 19 | 95.00 |
| | Total | 20 | 100.00 | 20 | 100.00 |
| Hormonal Fluctuation | Positive | 14 | 42.42 | 1 | 3.03 |
| | Negative | 19 | 57.58 | 32 | 96.97 |
| | Total | 33 | 100.00 | 33 | 100.00 |
| Gynecologic Disorder | Positive | 17 | 48.57 | 1 | 2.86 |
| | Negative | 18 | 51.43 | 34 | 97.14 |
| | Total | 35 | 100.00 | 35 | 100.00 |
| Vascular Disorder | Positive | 11 | 44.00 | 2 | 8.00 |
| | Negative | 14 | 56.00 | 23 | 92.00 |
| | Total | 25 | 100.00 | 25 | 100.00 |
| Chronic Immuno. Disorder | Positive | 22 | 52.38 | 5 | 11.90 |
| | Negative | 20 | 47.62 | 37 | 88.10 |
| | Total | 42 | 100.00 | 42 | 100.00 |

TABLE 14
Summary by Cancer Stage for Muc-1

| | |
|---|---|
| Best Cutoff | |
| from ROC plot | Mean + 2 * std |

| Stage | Result | N | % | N | % |
|---|---|---|---|---|---|
| 1 | Positive | 44 | 65.67 | 6 | 8.96 |
| | Negative | 23 | 34.33 | 61 | 91.04 |
| | Total | 67 | 100.00 | 67 | 100.00 |
| 2 | Positive | 27 | 40.91 | 3 | 4.55 |
| | Negative | 39 | 59.09 | 63 | 95.45 |
| | Total | 66 | 100.00 | 66 | 100.00 |
| 3 | Positive | 37 | 55.22 | 7 | 10.45 |
| | Negative | 30 | 44.78 | 60 | 89.55 |
| | Total | 67 | 100.00 | 67 | 100.00 |

### Biomarker: KLK-6

TABLE 15
Summary by Age Group for KLK-6

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 5 | 5.56 | 8 | 7.41 | 4 | 4.44 | 4 | 3.70 |
| | Negative | 85 | 94.44 | 100 | 92.59 | 86 | 95.56 | 104 | 96.30 |
| | Total | 90 | 100.00 | 108 | 100.00 | 90 | 100.00 | 108 | 100.00 |
| NORMAL | Positive | 6 | 2.99 | 11 | 5.64 | 2 | 1.00 | 5 | 2.56 |
| | Negative | 195 | 97.01 | 184 | 94.36 | 199 | 99.00 | 190 | 97.44 |
| | Total | 201 | 100.00 | 195 | 100.00 | 201 | 100.00 | 195 | 100.00 |
| OvCA | Positive | 10 | 10.00 | 13 | 13.00 | 4 | 4.00 | 6 | 6.00 |
| | Negative | 90 | 90.00 | 87 | 87.00 | 96 | 96.00 | 94 | 94.00 |
| | Total | 100 | 100.00 | 100 | 100.00 | 100 | 100.00 | 100 | 100.00 |

TABLE 16
Summary by Interfering Substance/Pathology Category for KLK-6

| | | Best Cutoff | | | |
|---|---|---|---|---|---|
| | | from ROC plot | | Mean + 2 * std | |
| Group | Result | N | % | N | % |
| Tumors | Positive | 5 | 11.11 | 4 | 8.89 |
| | Negative | 40 | 88.89 | 41 | 91.11 |
| | Total | 45 | 100.00 | 45 | 100.00 |
| Metabolic Disorder | Positive | 0 | 0.00 | 0 | 0.00 |
| | Negative | 20 | 100.00 | 20 | 100.00 |
| | Total | 20 | 100.00 | 20 | 100.00 |
| Hormonal Fluctuation | Positive | 0 | 0.00 | 0 | 0.00 |
| | Negative | 33 | 100.00 | 33 | 100.00 |
| | Total | 33 | 100.00 | 33 | 100.00 |
| Gynecologic Disorder | Positive | 3 | 8.57 | 2 | 5.71 |
| | Negative | 32 | 91.43 | 33 | 94.29 |
| | Total | 35 | 100.00 | 35 | 100.00 |
| Vascular Disorder | Positive | 3 | 12.00 | 1 | 4.00 |
| | Negative | 22 | 88.00 | 24 | 96.00 |
| | Total | 25 | 100.00 | 25 | 100.00 |
| Chronic Immuno. Disorder | Positive | 2 | 4.76 | 1 | 2.38 |
| | Negative | 40 | 95.24 | 41 | 97.62 |
| | Total | 42 | 100.00 | 42 | 100.00 |

TABLE 17
Summary by Cancer Stage Group for KLK-6

| | |
|---|---|
| Best Cutoff | |
| from ROC plot | Mean + 2 * std |

| Stage | Result | N | % | N | % |
|---|---|---|---|---|---|
| 1 | Positive | 10 | 14.93 | 4 | 5.97 |
| | Negative | 57 | 85.07 | 63 | 94.03 |
| | Total | 67 | 100.00 | 67 | 100.00 |
| 2 | Positive | 7 | 10.61 | 3 | 4.55 |
| | Negative | 59 | 89.39 | 63 | 95.45 |
| | Total | 66 | 100.00 | 66 | 100.00 |
| 3 | Positive | 6 | 8.96 | 3 | 4.48 |
| | Negative | 61 | 91.04 | 64 | 95.52 |
| | Total | 67 | 100.00 | 67 | 100.00 |

### Biomarker: KLK-10

TABLE 18
Summary by Age Group for KLK-10

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 56 | 62.22 | 51 | 47.22 | 7 | 7.78 | 6 | 5.56 |
| | Negative | 34 | 37.78 | 57 | 52.78 | 83 | 92.22 | 102 | 94.44 |
| | Total | 90 | 100.00 | 108 | 100.00 | 90 | 100.00 | 108 | 100.00 |
| NORMAL | Positive | 92 | 45.77 | 89 | 45.64 | 4 | 1.99 | 2 | 1.03 |
| | Negative | 109 | 54.23 | 106 | 54.36 | 197 | 98.01 | 193 | 98.97 |
| | Total | 201 | 100.00 | 195 | 100.00 | 201 | 100.00 | 195 | 100.00 |
| OvCA | Positive | 63 | 63.00 | 53 | 53.00 | 9 | 9.00 | 7 | 7.00 |
| | Negative | 37 | 37.00 | 47 | 47.00 | 91 | 91.00 | 93 | 93.00 |
| | Total | 100 | 100.00 | 100 | 100.00 | 100 | 100.00 | 100 | 100.00 |

TABLE 19
Summary by Interfering Substance/Pathology Group for KLK-10

| | | Best Cutoff | | | |
|---|---|---|---|---|---|
| | | from ROC plot | | Mean + 2 * std | |
| Group | Result | N | % | N | % |
| Tumors | Positive | 28 | 62.22 | 3 | 6.67 |
| | Negative | 17 | 37.78 | 42 | 93.33 |
| | Total | 45 | 100.00 | 45 | 100.00 |
| Metabolic Disorder | Positive | 10 | 50.00 | 0 | 0.00 |
| | Negative | 10 | 50.00 | 20 | 100.00 |
| | Total | 20 | 100.00 | 20 | 100.00 |
| Hormonal Fluctuation | Positive | 14 | 42.42 | 0 | 0.00 |
| | Negative | 19 | 57.58 | 33 | 100.00 |
| | Total | 33 | 100.00 | 33 | 100.00 |
| Gynecologic Disorder | Positive | 23 | 65.71 | 3 | 8.57 |
| | Negative | 12 | 34.29 | 32 | 91.43 |
| | Total | 35 | 100.00 | 35 | 100.00 |
| Vascular Disorder | Positive | 11 | 44.00 | 1 | 4.00 |
| | Negative | 14 | 56.00 | 24 | 96.00 |
| | Total | 25 | 100.00 | 25 | 100.00 |
| Chronic Immuno. Disorder | Positive | 23 | 54.76 | 6 | 14.29 |
| | Negative | 19 | 45.24 | 36 | 85.71 |
| | Total | 42 | 100.00 | 42 | 100.00 |

TABLE 20
Summary by Cancer Stage Group for KLK-10

| | |
|---|---|
| Best Cutoff from | |
| ROC plot | Mean + 2 * std |

| Stage | Result | N | % | N | % |
|---|---|---|---|---|---|
| 1 | Positive | 37 | 55.22 | 8 | 11.94 |
| | Negative | 30 | 44.78 | 59 | 88.06 |
| | Total | 67 | 100.00 | 67 | 100.00 |
| 2 | Positive | 37 | 56.06 | 3 | 4.55 |
| | Negative | 29 | 43.94 | 63 | 95.45 |
| | Total | 66 | 100.00 | 66 | 100.00 |
| 3 | Positive | 42 | 62.69 | 5 | 7.46 |
| | Negative | 25 | 37.31 | 62 | 92.54 |
| | Total | 67 | 100.00 | 67 | 100.00 |

### Biomarker: PAI-1

TABLE 21
Summary by Age Group for PAI-1

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 52 | 57.78 | 67 | 62.04 | 12 | 13.33 | 10 | 9.26 |
| | Negative | 38 | 42.22 | 41 | 37.96 | 78 | 86.67 | 98 | 90.74 |
| | Total | 90 | 100.00 | 108 | 100.00 | 90 | 100.00 | 108 | 100.00 |
| NORMAL | Positive | 43 | 21.39 | 52 | 26.67 | 5 | 2.49 | 10 | 5.13 |
| | Negative | 158 | 78.61 | 143 | 73.33 | 196 | 97.51 | 185 | 94.87 |
| | Total | 201 | 100.00 | 195 | 100.00 | 201 | 100.00 | 195 | 100.00 |
| OvCA | Positive | 71 | 71.00 | 62 | 62.00 | 19 | 19.00 | 11 | 11.00 |
| | Negative | 29 | 29.00 | 38 | 38.00 | 81 | 81.00 | 89 | 89.00 |
| | Total | 100 | 100.00 | 100 | 100.00 | 100 | 100.00 | 100 | 100.00 |

TABLE 22
Summary by Interfering Substance/Pathology Group for PAI-1

| | | Best Cutoff | | | |
|---|---|---|---|---|---|
| | | from ROC | | | |
| | | plot | | Mean + 2 * std | |
| Group | Result | N | % | N | % |
| Tumors | Positive | 17 | 37.78 | 4 | 8.89 |
| | Negative | 28 | 62.22 | 41 | 91.11 |
| | Total | 45 | 100.00 | 45 | 100.00 |
| Metabolic Disorder | Positive | 16 | 80.00 | 4 | 20.00 |
| | Negative | 4 | 20.00 | 16 | 80.00 |
| | Total | 20 | 100.00 | 20 | 100.00 |
| Hormonal Fluctuation | Positive | 24 | 72.73 | 5 | 15.15 |
| | Negative | 9 | 27.27 | 28 | 84.85 |
| | Total | 33 | 100.00 | 33 | 100.00 |
| Gynecologic Disorder | Positive | 17 | 48.57 | 2 | 5.71 |
| | Negative | 18 | 51.43 | 33 | 94.29 |
| | Total | 35 | 100.00 | 35 | 100.00 |
| Vascular Disorder | Positive | 17 | 68.00 | 1 | 4.00 |
| | Negative | 8 | 32.00 | 24 | 96.00 |
| | Total | 25 | 100.00 | 25 | 100.00 |
| Chronic Immuno. | Positive | 30 | 71.43 | 6 | 14.29 |
| Disorder | Negative | 12 | 28.57 | 36 | 85.71 |
| | Total | 42 | 100.00 | 42 | 100.00 |

TABLE 23
Summary by Cancer Stage Group for PAI-1

| | |
|---|---|
| Best Cutoff from | |
| ROC plot | Mean + 2 * std |

| Stage | Result | N | % | N | % |
|---|---|---|---|---|---|
| 1 | Positive | 54 | 80.60 | 15 | 22.39 |
| | Negative | 13 | 19.40 | 52 | 77.61 |
| | Total | 67 | 100.00 | 67 | 100.00 |
| 2 | Positive | 33 | 50.00 | 4 | 6.06 |
| | Negative | 33 | 50.00 | 62 | 93.94 |
| | Total | 66 | 100.00 | 66 | 100.00 |
| 3 | Positive | 46 | 68.66 | 11 | 16.42 |
| | Negative | 21 | 31.34 | 56 | 83.58 |
| | Total | 67 | 100.00 | 67 | 100.0 |

### Biomarker: CTHRC1

TABLE 24
Summary by Age Group for CTHRC1

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 12 | 13.33 | 35 | 32.41 | 6 | 6.67 | 11 | 10.19 |
| | Negative | 78 | 86.67 | 73 | 67.59 | 84 | 93.33 | 97 | 89.81 |
| | Total | 90 | 100.00 | 108 | 100.00 | 90 | 100.00 | 108 | 100.00 |
| NORMAL | Positive | 20 | 9.95 | 40 | 20.51 | 4 | 1.99 | 4 | 2.05 |
| | Negative | 181 | 90.05 | 155 | 79.49 | 197 | 98.01 | 191 | 97.95 |
| | Total | 201 | 100.00 | 195 | 100.00 | 201 | 100.00 | 195 | 100.00 |
| OvCA | Positive | 24 | 24.00 | 35 | 35.00 | 10 | 10.00 | 15 | 15.00 |
| | Negative | 76 | 76.00 | 65 | 65.00 | 90 | 90.00 | 85 | 85.00 |
| | Total | 100 | 100.00 | 100 | 100.00 | 100 | 100.00 | 100 | 100.00 |

TABLE 25
Summary by Interfering Substance/Pathology Group for CTHRC1

| | | Best Cutoff from | | | |
|---|---|---|---|---|---|
| | | ROC plot | | Mean + 2 * std | |
| Group | Result | N | % | N | % |
| Tumors | Positive | 13 | 28.89 | 7 | 15.56 |
| | Negative | 32 | 71.11 | 38 | 84.44 |
| | Total | 45 | 100.00 | 45 | 100.00 |
| Metabolic Disorder | Positive | 2 | 10.00 | 0 | 0.00 |
| | Negative | 18 | 90.00 | 20 | 100.00 |
| | Total | 20 | 100.00 | 20 | 100.00 |
| Hormonal Fluctuation | Positive | 1 | 3.03 | 1 | 3.03 |
| | Negative | 32 | 96.97 | 32 | 96.97 |
| | Total | 33 | 100.00 | 33 | 100.00 |
| Gynecologic Disorder | Positive | 2 | 5.71 | 0 | 0.00 |
| | Negative | 33 | 94.29 | 35 | 100.00 |
| | Total | 35 | 100.00 | 35 | 100.00 |
| Vascular Disorder | Positive | 11 | 44.00 | 5 | 20.00 |
| | Negative | 14 | 56.00 | 20 | 80.00 |
| | Total | 25 | 100.00 | 25 | 100.00 |
| Chronic Immuno. | Positive | 18 | 42.86 | 4 | 9.52 |
| Disorder | Negative | 24 | 57.14 | 38 | 90.48 |
| | Total | 42 | 100.00 | 42 | 100.00 |

TABLE 26
Summary by Cancer Stage Group for CTHRC1

| | |
|---|---|
| Best Cutoff from | |
| ROC plot | Mean + 2 * std |

| Stage | Result | N | % | N | % |
|---|---|---|---|---|---|
| 1 | Positive | 16 | 23.88 | 7 | 10.45 |
| | Negative | 51 | 76.12 | 60 | 89.55 |
| | Total | 67 | 100.00 | 67 | 100.00 |
| 2 | Positive | 19 | 28.79 | 8 | 12.12 |
| | Negative | 47 | 71.21 | 58 | 87.88 |
| | Total | 66 | 100.00 | 66 | 100.00 |
| 3 | Positive | 24 | 35.82 | 10 | 14.93 |
| | Negative | 43 | 64.18 | 57 | 85.07 |
| | Total | 67 | 100.00 | 67 | 100.00 |

### Biomarker: SLPI

TABLE 27
Summary by Age Group for SLPI

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 27 | 30.00 | 61 | 56.48 | 4 | 4.44 | 23 | 21.30 |
| | Negative | 63 | 70.00 | 47 | 43.52 | 86 | 95.56 | 85 | 78.70 |
| | Total | 90 | 100.00 | 108 | 100.00 | 90 | 100.00 | 108 | 100.00 |
| NORMAL | Positive | 49 | 24.38 | 63 | 32.31 | 4 | 1.99 | 3 | 1.54 |
| | Negative | 152 | 75.62 | 132 | 67.69 | 197 | 98.01 | 192 | 98.46 |
| | Total | 201 | 100.00 | 195 | 100.00 | 201 | 100.00 | 195 | 100.00 |
| OvCA | Positive | 53 | 53.00 | 67 | 67.00 | 14 | 14.00 | 16 | 16.00 |
| | Negative | 47 | 47.00 | 33 | 33.00 | 86 | 86.00 | 84 | 84.00 |
| | Total | 100 | 100.00 | 100 | 100.00 | 100 | 100.00 | 100 | 100.00 |

TABLE 28
Summary by Interfering Substance/Pathology Group for SLPI

| | | Best Cutoff from | | | |
|---|---|---|---|---|---|
| | | ROC plot | | Mean + 2 * std | |
| Group | Result | N | % | N | % |
| Tumors | Positive | 20 | 44.44 | 7 | 15.56 |
| | Negative | 25 | 55.56 | 38 | 84.44 |
| | Total | 45 | 100.00 | 45 | 100.00 |
| Metabolic Disorder | Positive | 10 | 50.00 | 1 | 5.00 |
| | Negative | 10 | 50.00 | 19 | 95.00 |
| | Total | 20 | 100.00 | 20 | 100.00 |
| Hormonal Fluctuation | Positive | 6 | 18.18 | 0 | 0.00 |
| | Negative | 27 | 81.82 | 33 | 100.00 |
| | Total | 33 | 100.00 | 33 | 100.00 |
| Gynecologic Disorder | Positive | 12 | 34.29 | 4 | 11.43 |
| | Negative | 23 | 65.71 | 31 | 88.57 |
| | Total | 35 | 100.00 | 35 | 100.00 |
| Vascular Disorder | Positive | 16 | 64.00 | 9 | 36.00 |
| | Negative | 9 | 36.00 | 16 | 64.00 |
| | Total | 25 | 100.00 | 25 | 100.00 |
| Chronic Immuno. | Positive | 26 | 61.90 | 7 | 16.67 |
| Disorder | Negative | 16 | 38.10 | 35 | 83.33 |
| | Total | 42 | 100.00 | 42 | 100.00 |

TABLE 29
Summary by Cancer Stage Group for SLPI

| | |
|---|---|
| Best Cutoff from | |
| ROC plot | Mean + 2 * std |

| Stage | Result | N | % | N | % |
|---|---|---|---|---|---|
| 1 | Positive | 34 | 50.75 | 9 | 13.43 |
| | Negative | 33 | 49.25 | 58 | 86.57 |
| | Total | 67 | 100.00 | 67 | 100.00 |
| 2 | Positive | 38 | 57.58 | 4 | 6.06 |
| | Negative | 28 | 42.42 | 62 | 93.94 |
| | Total | 66 | 100.00 | 66 | 100.00 |
| 3 | Positive | 48 | 71.64 | 17 | 25.37 |
| | Negative | 19 | 28.36 | 50 | 74.63 |
| | Total | 67 | 100.00 | 67 | 100.00 |

### Biomarker: Inhibin A

TABLE 30
Summary by Age Group for Inhibin

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 53 | 58.89 | 11 | 10.19 | 48 | 53.33 | 7 | 6.48 |
| | Negative | 37 | 41.11 | 97 | 89.81 | 42 | 46.67 | 101 | 93.52 |
| | Total | 90 | 100.00 | 108 | 100.00 | 90 | 100.00 | 108 | 100.00 |
| NORMAL | Positive | 94 | 46.77 | 25 | 12.82 | 83 | 41.29 | 14 | 7.18 |
| | Negative | 107 | 53.23 | 170 | 87.18 | 118 | 58.71 | 181 | 92.82 |
| | Total | 201 | 100.00 | 195 | 100.00 | 201 | 100.00 | 195 | 100.00 |
| OvCA | Positive | 42 | 42.00 | 41 | 41.00 | 32 | 32.00 | 36 | 36.00 |
| | Negative | 58 | 58.00 | 59 | 59.00 | 68 | 68.00 | 64 | 64.00 |
| | Total | 100 | 100.00 | 100 | 100.00 | 100 | 100.00 | 100 | 100.00 |

TABLE 31
Summary by Interfering Substance/Pathology Group for Inhibin

| | | Best Cutoff from | | | |
|---|---|---|---|---|---|
| | | ROC plot | | Mean + 2 * std | |
| Group | Result | N | % | N | % |
| Tumors | Positive | 4 | 8.89 | 2 | 4.44 |
| | Negative | 41 | 91.11 | 43 | 95.56 |
| | Total | 45 | 100.00 | 45 | 100.00 |
| Metabolic Disorder | Positive | 8 | 40.00 | 8 | 40.00 |
| | Negative | 12 | 60.00 | 12 | 60.00 |
| | Total | 20 | 100.00 | 20 | 100.00 |
| Hormonal Fluctuation | Positive | 16 | 48.48 | 16 | 48.48 |
| | Negative | 17 | 51.52 | 17 | 51.52 |
| | Total | 33 | 100.00 | 33 | 100.00 |
| Gynecologic Disorder | Positive | 27 | 77.14 | 25 | 71.43 |
| | Negative | 8 | 22.86 | 10 | 28.57 |
| | Total | 35 | 100.00 | 35 | 100.00 |
| Vascular Disorder | Positive | 3 | 12.00 | 2 | 8.00 |
| | Negative | 22 | 88.00 | 23 | 92.00 |
| | Total | 25 | 100.00 | 25 | 100.00 |
| Chronic Immuno. | Positive | 8 | 19.05 | 4 | 9.52 |
| Disorder | Negative | 34 | 80.95 | 38 | 90.48 |
| | Total | 42 | 100.00 | 42 | 100.00 |

TABLE 32
Summary by Cancer Stage Group for Inhibin

| | |
|---|---|
| Best Cutoff from | |
| ROC plot | Mean + 2 * std |

| Stage | Result | N | % | N | % |
|---|---|---|---|---|---|
| 1 | Positive | 25 | 37.31 | 20 | 29.85 |
| | Negative | 42 | 62.69 | 47 | 70.15 |
| | Total | 67 | 100.00 | 67 | 100.00 |
| 2 | Positive | 35 | 53.03 | 32 | 48.48 |
| | Negative | 31 | 46.97 | 34 | 51.52 |
| | Total | 66 | 100.00 | 66 | 100.00 |
| 3 | Positive | 23 | 34.33 | 16 | 23.88 |
| | Negative | 44 | 65.67 | 51 | 76.12 |
| | Total | 67 | 100.00 | 67 | 100.00 |

### Biomarker: Glycodelin

TABLE 33
Summary by Age Group for Glycodelin

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 53 | 58.89 | 18 | 16.67 | 36 | 40.00 | 10 | 9.26 |
| | Negative | 37 | 41.11 | 90 | 83.33 | 54 | 60.00 | 98 | 90.74 |
| | Total | 90 | 100.00 | 108 | 100.00 | 90 | 100.00 | 108 | 100.00 |
| NORMAL | Positive | 73 | 36.32 | 10 | 5.13 | 56 | 27.86 | 3 | 1.54 |
| | Negative | 128 | 63.68 | 185 | 94.87 | 145 | 72.14 | 192 | 98.46 |
| | Total | 201 | 100.00 | 195 | 100.00 | 201 | 100.00 | 195 | 100.00 |
| OvCA | Positive | 65 | 65.00 | 62 | 62.00 | 49 | 49.00 | 52 | 52.00 |
| | Negative | 35 | 35.00 | 38 | 38.00 | 51 | 51.00 | 48 | 48.00 |
| | Total | 100 | 100.00 | 100 | 100.00 | 100 | 100.00 | 100 | 100.00 |

TABLE 34
Summary by Interfering Substance/Pathology Group for Glycodelin

| | | Best Cutoff from | | | |
|---|---|---|---|---|---|
| | | ROC plot | | Mean + 2 * std | |
| Group | Result | N | % | N | % |
| Tumors | Positive | 13 | 28.89 | 6 | 13.33 |
| | Negative | 32 | 71.11 | 39 | 86.67 |
| | Total | 45 | 100.00 | 45 | 100.00 |
| Metabolic Disorder | Positive | 9 | 45.00 | 5 | 25.00 |
| | Negative | 11 | 55.00 | 15 | 75.00 |
| | Total | 20 | 100.00 | 20 | 100.00 |
| Hormonal Fluctuation | Positive | 18 | 54.55 | 15 | 45.45 |
| | Negative | 15 | 45.45 | 18 | 54.55 |
| | Total | 33 | 100.00 | 33 | 100.00 |
| Gynecologic Disorder | Positive | 21 | 60.00 | 14 | 40.00 |
| | Negative | 14 | 40.00 | 21 | 60.00 |
| | Total | 35 | 100.00 | 35 | 100.00 |
| Vascular Disorder | Positive | 1 | 4.00 | 1 | 4.00 |
| | Negative | 24 | 96.00 | 24 | 96.00 |
| | Total | 25 | 100.00 | 25 | 100.00 |
| Chronic Immuno. | Positive | 9 | 21.43 | 5 | 11.90 |
| Disorder | Negative | 33 | 78.57 | 37 | 88.10 |
| | Total | 42 | 100.00 | 42 | 100.00 |

TABLE 35
Summary by Cancer Stage Group for Glycodelin

| | |
|---|---|
| Best Cutoff | |
| from | |
| ROC plot | Mean + 2 * std |

| Stage | Result | N | % | N | % |
|---|---|---|---|---|---|
| 1 | Positive | 43 | 64.18 | 34 | 50.75 |
| | Negative | 24 | 35.82 | 33 | 49.25 |
| | Total | 67 | 100.00 | 67 | 100.00 |
| 2 | Positive | 41 | 62.12 | 37 | 56.06 |
| | Negative | 25 | 37.88 | 29 | 43.94 |
| | Total | 66 | 100.00 | 66 | 100.00 |
| 3 | Positive | 43 | 64.18 | 30 | 44.78 |
| | Negative | 24 | 35.82 | 37 | 55.22 |
| | Total | 67 | 100.00 | 67 | 100.00 |

### Biomarker: MMP-7

TABLE 36
Summary by Age Group for MMP-7

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 19 | 21.11 | 78 | 72.22 | 9 | 10.00 | 44 | 40.74 |
| | Negative | 71 | 78.89 | 30 | 27.78 | 81 | 90.00 | 64 | 59.26 |
| | Total | 90 | 100.00 | 108 | 100.00 | 90 | 100.00 | 108 | 100.00 |
| NORMAL | Positive | 12 | 5.97 | 30 | 15.38 | 2 | 1.00 | 7 | 3.59 |
| | Negative | 189 | 94.03 | 165 | 84.62 | 199 | 99.00 | 188 | 96.41 |
| | Total | 201 | 100.00 | 195 | 100.00 | 201 | 100.00 | 195 | 100.00 |
| OvCA | Positive | 50 | 50.00 | 53 | 53.00 | 28 | 28.00 | 33 | 33.00 |
| | Negative | 50 | 50.00 | 47 | 47.00 | 72 | 72.00 | 67 | 67.00 |
| | Total | 100 | 100.00 | 100 | 100.00 | 100 | 100.00 | 100 | 100.00 |

TABLE 37
Summary by Interfering Substance/Pathology Group for MMP-7

| | | Best Cutoff from | | | |
|---|---|---|---|---|---|
| | | ROC plot | | Mean + 2 * std | |
| Group | Result | N | % | N | % |
| Tumors | Positive | 27 | 60.00 | 15 | 33.33 |
| | Negative | 18 | 40.00 | 30 | 66.67 |
| | Total | 45 | 100.00 | 45 | 100.00 |
| Metabolic Disorder | Positive | 8 | 40.00 | 5 | 25.00 |
| | Negative | 12 | 60.00 | 15 | 75.00 |
| | Total | 20 | 100.00 | 20 | 100.00 |
| Hormonal Fluctuation | Positive | 3 | 9.09 | 0 | 0.00 |
| | Negative | 30 | 90.91 | 33 | 100.00 |
| | Total | 33 | 100.00 | 33 | 100.00 |
| Gynecologic Disorder | Positive | 8 | 22.86 | 3 | 8.57 |
| | Negative | 27 | 77.14 | 32 | 91.43 |
| | Total | 35 | 100.00 | 35 | 100.00 |
| Vascular Disorder | Positive | 22 | 88.00 | 10 | 40.00 |
| | Negative | 3 | 12.00 | 15 | 60.00 |
| | Total | 25 | 100.00 | 25 | 100.00 |
| Chronic Immuno. | Positive | 29 | 69.05 | 20 | 47.62 |
| Disorder | Negative | 13 | 30.95 | 22 | 52.38 |
| | Total | 42 | 100.00 | 42 | 100.00 |

TABLE 38
Summary by Cancer Stage Group for MMP-7

| | |
|---|---|
| Best Cutoff from | |
| ROC plot | Mean + 2 * std |

| Stage | Result | N | % | N | % |
|---|---|---|---|---|---|
| 1 | Positive | 33 | 49.25 | 20 | 29.85 |
| | Negative | 34 | 50.75 | 47 | 70.15 |
| | Total | 67 | 100.00 | 67 | 100.00 |
| 2 | Positive | 28 | 42.42 | 11 | 16.67 |
| | Negative | 38 | 57.58 | 55 | 83.33 |
| | Total | 66 | 100.00 | 66 | 100.00 |
| 3 | Positive | 42 | 62.69 | 30 | 44.78 |
| | Negative | 25 | 37.31 | 37 | 55.22 |
| | Total | 67 | 100.00 | 67 | 100.00 |

### Biomarker: PLAU-R

TABLE 39
Summary by Age Group for PLAU-R

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 50 | 55.56 | 71 | 65.74 | 24 | 26.67 | 56 | 51.85 |
| | Negative | 40 | 44.44 | 37 | 34.26 | 66 | 73.33 | 52 | 48.15 |
| | Total | 90 | 100.00 | 108 | 100.00 | 90 | 100.00 | 108 | 100.00 |
| NORMAL | Positive | 35 | 17.41 | 61 | 31.28 | 16 | 7.96 | 23 | 11.79 |
| | Negative | 166 | 82.59 | 134 | 68.72 | 185 | 92.04 | 172 | 88.21 |
| | Total | 201 | 100.00 | 195 | 100.00 | 201 | 100.00 | 195 | 100.00 |
| OvCA | Positive | 71 | 71.00 | 72 | 72.00 | 44 | 44.00 | 50 | 50.00 |
| | Negative | 29 | 29.00 | 28 | 28.00 | 56 | 56.00 | 50 | 50.00 |
| | Total | 100 | 100.00 | 100 | 100.00 | 100 | 100.00 | 100 | 100.00 |

TABLE 40
Summary by Interfering Substance/Pathology Group for PLAU-R

| | | Best Cutoff from | | | |
|---|---|---|---|---|---|
| | | ROC plot | | Mean + 2 * std | |
| Group | Result | N | % | N | % |
| Tumors | Positive | 30 | 66.67 | 20 | 44.44 |
| | Negative | 15 | 33.33 | 25 | 55.56 |
| | Total | 45 | 100.00 | 45 | 100.00 |
| Metabolic Disorder | Positive | 13 | 65.00 | 11 | 55.00 |
| | Negative | 7 | 35.00 | 9 | 45.00 |
| | Total | 20 | 100.00 | 20 | 100.00 |
| Hormonal Fluctuation | Positive | 13 | 39.39 | 1 | 3.03 |
| | Negative | 20 | 60.61 | 32 | 96.97 |
| | Total | 33 | 100.00 | 33 | 100.00 |
| Gynecologic Disorder | Positive | 15 | 42.86 | 9 | 25.71 |
| | Negative | 20 | 57.14 | 26 | 74.29 |
| | Total | 35 | 100.00 | 35 | 100.00 |
| Vascular Disorder | Positive | 18 | 72.00 | 11 | 44.00 |
| | Negative | 7 | 28.00 | 14 | 56.00 |
| | Total | 25 | 100.00 | 25 | 100.00 |
| Chronic Immuno. | Positive | 32 | 76.19 | 28 | 66.67 |
| Disorder | Negative | 10 | 23.81 | 14 | 33.33 |
| | Total | 42 | 100.00 | 42 | 100.00 |

TABLE 41
Summary by Cancer Stage Group for PLAU-R

| | |
|---|---|
| Best Cutoff from | |
| ROC plot | Mean + 2 * std |

| Stage | Result | N | % | N | % |
|---|---|---|---|---|---|
| 1 | Positive | 53 | 79.10 | 30 | 44.78 |
| | Negative | 14 | 20.90 | 37 | 55.22 |
| | Total | 67 | 100.00 | 67 | 100.00 |
| 2 | Positive | 40 | 60.61 | 29 | 43.94 |
| | Negative | 26 | 39.39 | 37 | 56.06 |
| | Total | 66 | 100.00 | 66 | 100.00 |
| 3 | Positive | 50 | 74.63 | 35 | 52.24 |
| | Negative | 17 | 25.37 | 32 | 47.76 |
| | Total | 67 | 100.00 | 67 | 100.00 |

### Biomarker: Prolactin

TABLE 42
Summary by Age Group for Prolactin

| | | Best Cutoff from ROC plot | | | | Mean + 2 * std | | | |
|---|---|---|---|---|---|---|---|---|---|
| | | Age <= 55 | | Age > 55 | | Age <= 55 | | Age > 55 | |
| Group | Result | N | % | N | % | N | % | N | % |
| IP | Positive | 50 | 55.56 | 49 | 45.37 | 9 | 10.00 | 6 | 5.56 |
| | Negative | 40 | 44.44 | 59 | 54.63 | 81 | 90.00 | 102 | 94.44 |
| | Total | 90 | 100.00 | 108 | 100.00 | 90 | 100.00 | 108 | 100.00 |
| NORMAL | Positive | 92 | 45.77 | 54 | 27.69 | 6 | 2.99 | 3 | 1.54 |
| | Negative | 109 | 54.23 | 141 | 72.31 | 195 | 97.01 | 192 | 98.46 |
| | Total | 201 | 100.00 | 195 | 100.00 | 201 | 100.00 | 195 | 100.00 |
| OvCA | Positive | 64 | 64.00 | 48 | 48.00 | 5 | 5.00 | 2 | 2.00 |
| | Negative | 36 | 36.00 | 52 | 52.00 | 95 | 95.00 | 98 | 98.00 |
| | Total | 100 | 100.00 | 100 | 100.00 | 100 | 100.00 | 100 | 100.00 |

TABLE 43
Summary by Interfering Substance/Pathology Group for Prolactin

| | | Best Cutoff from | | | |
|---|---|---|---|---|---|
| | | ROC plot | | Mean + 2 * std | |
| Group | Result | N | % | N | % |
| Tumors | Positive | 17 | 37.78 | 3 | 6.67 |
| | Negative | 28 | 62.22 | 42 | 93.33 |
| | Total | 45 | 100.00 | 45 | 100.00 |
| Metabolic Disorder | Positive | 4 | 20.00 | 2 | 10.00 |
| | Negative | 16 | 80.00 | 18 | 90.00 |
| | Total | 20 | 100.00 | 20 | 100.00 |
| Hormonal Fluctuation | Positive | 23 | 69.70 | 4 | 12.12 |
| | Negative | 10 | 30.30 | 29 | 87.88 |
| | Total | 33 | 100.00 | 33 | 100.00 |
| Gynecologic Disorder | Positive | 18 | 51.43 | 3 | 8.57 |
| | Negative | 17 | 48.57 | 32 | 91.43 |
| | Total | 35 | 100.00 | 35 | 100.00 |
| Vascular Disorder | Positive | 13 | 52.00 | 2 | 8.00 |
| | Negative | 12 | 48.00 | 23 | 92.00 |
| | Total | 25 | 100.00 | 25 | 100.00 |
| Chronic Immuno. | Positive | 26 | 61.90 | 2 | 4.76 |
| Disorder | Negative | 16 | 38.10 | 40 | 95.24 |
| | Total | 42 | 100.00 | 42 | 100.00 |

TABLE 44
Summary by Cancer Stage Group for Prolactin

| | |
|---|---|
| Best Cutoff from | |
| ROC plot | Mean + 2 * std |

| Stage | Result | N | % | N | % |
|---|---|---|---|---|---|
| 1 | Positive | 42 | 62.69 | 2 | 2.99 |
| | Negative | 25 | 37.31 | 65 | 97.01 |
| | Total | 67 | 100.00 | 67 | 100.00 |
| 2 | Positive | 30 | 45.45 | 2 | 3.03 |
| | Negative | 36 | 54.55 | 64 | 96.97 |
| | Total | 66 | 100.00 | 66 | 100.00 |
| 3 | Positive | 40 | 59.70 | 3 | 4.48 |
| | Negative | 27 | 40.30 | 64 | 95.52 |
| | Total | 67 | 100.00 | 67 | 100.00 |

### Biomarker: CA125 (Lab Corp Data)

TABLE 45
Summary by Age Group for CA125 (LabCorp Data)

Cutoff at 35

| | |
|---|---|
| Age <= 55 | Age > 55 |

| Group | Result | N | % | N | % |
|---|---|---|---|---|---|
| IP | Positive | 13 | 14.94 | 6 | 5.66 |
| | Negative | 74 | 85.06 | 100 | 94.34 |
| | Total | 87 | 100.00 | 106 | 100.00 |
| NORMAL | Positive | 5 | 2.51 | 1 | 0.52 |
| | Negative | 194 | 97.49 | 193 | 99.48 |
| | Total | 199 | 100.00 | 194 | 100.00 |
| OvCA | Positive | 39 | 39.39 | 60 | 60.61 |
| | Negative | 60 | 60.61 | 39 | 39.39 |
| | Total | 99 | 100.00 | 99 | 100.00 |

TABLE 46
Summary by Interfering Substance/Pathology Group for CA125
(LabCorp data)

Cutoff at 35

| Group | Result | N | % |
|---|---|---|---|
| Tumors | Positive | 5 | 11.11 |
| | Negative | 40 | 88.89 |
| | Total | 45 | 100.00 |
| Metabolic Disorder | Positive | 1 | 5.56 |
| | Negative | 17 | 94.44 |
| | Total | 18 | 100.00 |
| Hormonal Fluctuation | Positive | 7 | 21.21 |
| | Negative | 26 | 78.79 |
| | Total | 33 | 100.00 |
| Gynecologic Disorder | Positive | 4 | 12.50 |
| | Negative | 28 | 87.50 |
| | Total | 32 | 100.00 |
| Vascular Disorder | Positive | 0 | 0.00 |
| | Negative | 25 | 100.00 |
| | Total | 25 | 100.00 |
| Chronic Immuno. | Positive | 2 | 4.76 |
| Disorder | Negative | 40 | 95.24 |
| | Total | 42 | 100.00 |

TABLE 47
Summary by Cancer Stage Group for CA 125 (LabCorp data)

Cutoff at 35

| Stage | Result | N | % |
|---|---|---|---|
| 1 | Positive | 24 | 35.82 |
| | Negative | 43 | 64.18 |
| | Total | 67 | 100.00 |
| 2 | Positive | 37 | 56.06 |
| | Negative | 29 | 43.94 |
| | Total | 66 | 100.00 |
| 3 | Positive | 38 | 58.46 |
| | Negative | 27 | 41.54 |
| | Total | 65 | 100.00 |

Summaries of the sensitivity of the thirteen biomarkers analyzed are presented in Tables 48-50, based on tumor stage, normal 2-standard deviation limits, or “best fit,” respectively.

TABLE 48
Individual marker sensitivity summary by tumor stage
(Based on best cutoff)

| | Stage 1 | Stage 2 | Stage 3 |
|---|---|---|---|
| Marker | % pos | % pos | % pos |
| HE4 | 85.1 | 80.3 | 92.5 |
| Glycodelin | 64.2 | 62.1 | 64.2 |
| MMP7 | 49.3 | 42.4 | 62.7 |
| SLPI | 50.8 | 57.6 | 71.6 |
| PAI-1 | 80.6 | 50.0 | 68.7 |
| MUC-1 | 65.7 | 40.9 | 55.2 |
| CA125 | 35.8 | 56.1 | 58.5 |
| Plau-R | 79.1 | 60.6 | 74.6 |
| Inhibin A | 37.3 | 53.0 | 34.3 |
| CTHRC1 | 23.9 | 28.8 | 35.8 |
| KLK6 | 14.9 | 10.6 | 9.0 |
| KLK10 | 55.2 | 56.1 | 62.7 |
| Prolactin | 62.7 | 45.5 | 59.7 |

TABLE 49
Individual Marker Sensitivity Summary (based on Normal 2SD limit)

| Marker | Sensitivity | Specificity |
|---|---|---|
| HE4 | 66.5 | 96 |
| Glycodelin | 50.5 | 85.3 |
| MMP7 | 30.5 | 97.7 |
| SLPI | 15.0 | 98.3 |
| Plau-R | 47.0 | 90.1 |
| MUC1 | 8.0 | 98.5 |
| Inhibin A | 34.0 | 75.8 |
| PAI-1 | 15.0 | 96.2 |
| CA125 | 50.0 | 98.5 |
| (>=35 u/ml) | | |
| CTHRC1 | 12.5 | 98.0 |
| KLK6 | 5.0 | 98.2 |
| KLK10 | 8.0 | 98.5 |
| Prolactin | 3.5 | 97.7 |

TABLE 50
Individual Marker Sensitivity Summary (based on best cutoff)

| Marker | Sensitivity | Specificity |
|---|---|---|
| HE4 | 86.0 | 86.3 |
| Glycodelin | 63.5 | 79.0 |
| MMP7 | 51.5 | 89.4 |
| SLPI | 60.0 | 71.7 |
| Plau-R | 71.5 | 77.2 |
| MUC1 | 54.0 | 73.0 |
| Inhibin A | 40.7 | 70.3 |
| PAI-1 | 66.5 | 76.0 |
| CA125 | 50.5 | 98.2 |
| (>=35 u/ml) | | |
| CTHRC1 | 29.5 | 84.8 |
| KLK6 | 11.5 | 95.7 |
| KLK10 | 58.0 | 54.3 |
| Prolactin | 56.0 | 63.1 |

### Example 3

### Ovarian Biomarker Selection Study—Multiple Biomarker Analysis

The logistic regression method was used for selection of optimal biomarkers for this study. Using logistic regression, stepwise selection starts off by finding the biomarker that produces the largest R-square value with the dependent variable. Then, given that the ‘best’ biomarker is in the model, logistic regression finds that next best biomarker in terms of adding to the R-square. This process of finding a set of biomarkers that adds to the R-square is stopped when biomarkers can no longer add to the R-square, in accordance with certain statistical criteria. These sets of biomarkers are not unique because some of the biomarkers have the same predictive performance.

Based on one selected model, a ROC plot can be produced, which provides the relationship between the sensitivity and specificity rates. Therefore, an appropriate specificity and sensitivity can be chosen in order to obtain an expected PPV with accepted sensitivity.

Tables 51 and 52 provide several examples involving selection of a set of biomarkers with a higher specificity and an appropriate sensitivity with a PPV of approximately 10% and a combined sensitivity rate around 70%-80%, under the assumption that the population is 8,000,000 and the disease prevalence is 0.25%. Different target values for sensitivity, specificity, NPV and PPV can be obtained by selecting different assay cutoffs, different biomarker combinations, and different rules for combining the biomarkers. Biomarker performance was analyzed using the biomarker values as continuous variables (Table 51) and as categorical variables (Table 52).

By way of definition, when two assay steps are performed, the first assay step may be referred to as the “screen test,” and the second step may be referred to as the “reflex test.” “Best cutoff point” refers to the value obtained from the ROC plot for a particular biomarker that produces the best sensitivity and specificity. “Mean+2*std” refers to the average expression level plus twice the standard deviation, based on analysis of normal samples from patients not afflicted with ovarian cancer.

### Biomarker Performance as Continuous Variables

TABLE 51
Estimated Sensitivity, Specificity, PPV, and NPV Adjusted by Disease
Prevalence for Different Models (Biomarker values as continuous variables)

| | Marker Selected and | Screen/Reflex | Screen/Reflex | Combined Performance* | | | |
|---|---|---|---|---|---|---|---|
| Example | rule | Test Sensitivity | Test Specificity | Sensitivity | Specificity | PPV | NPV |

Age > 55

| | | | | | | | |
|---|---|---|---|---|---|---|---|
| 1 | Screen Test: | 90.2% | 62.2% | | | | |
| | If HE4 <=1.8 then | | | | | | |
| | test is negative | | | | | | |
| | Reflex Test: | 87% | 96% | 78.474% | 98.4880% | 11.5105% | 99.9453% |
| | If HE4 >1.8 then the | | | | | | |
| | following marker | | | | | | |
| | selected: | | | | | | |
| | CA125, GLY, PAI, | | | | | | |
| | Plau-R | | | | | | |
| | (actual value used**) | | | | | | |
| 2† | Marker selected: | 81.7% | 98% | 81.7% | 98% | 9.2873% | 99.9532% |
| | HE4, CA125, GLY, | | | | | | |
| | MMP-7, PAI, Plau-R | | | | | | |
| | (actual value used) | | | | | | |

*Assume the population = 8,000,000; prevalence = 0.25%, estimated cancer cases = 20,000 in the population.
**Actual value refers to the actual biomarker expression level obtained in the test
†Although all listed biomarkers were assayed in a single step, additional algorithms were applied to obtain the values for sensitivity, specificity, PPV, and NPV indicated in the table.

### Biomarker Performance as Categorical Variables

TABLE 52
Estimated Sensitivity, Specificity, PPV, and NPV Adjusted by Disease Prevalence for
Different Models (Biomarker Values as Categorical Variables)

| | Marker Selected and | Screen/Reflex | Screen/Reflex | Combined Performance* | | | |
|---|---|---|---|---|---|---|---|
| Example | rule | Test Sensitivity | Test Specificity | Sensitivity | Specificity | PPV | NPV |

Overall Sample

| | | | | | | | |
|---|---|---|---|---|---|---|---|
| 1 | Screen Test: | 91.9% | 73% | | | | |
| | If HE4 <=1.8 then | | | | | | |
| | test is negative | | | | | | |
| | Reflex Test: | 80% | 92.5% | 73.52% | 98.704% | 12.4479% | 99.9328% |
| | If HE4 >1.8 then the | | | | | | |
| | following marker | | | | | | |
| | selected: | | | | | | |
| | CA125, MUC1, | | | | | | |
| | GLY, PAI-1, Plau-R | | | | | | |
| | (Best cutoff points | | | | | | |
| | used) | | | | | | |

Age > 55

| | | | | | | | |
|---|---|---|---|---|---|---|---|
| 1 | Screen Test: | 90.2% | 62.2% | | | | |
| | If HE4 <=1.8 then | | | | | | |
| | test is negative | | | | | | |
| | Reflex Test: | 78% | 95.9% | 70.356% | 98.4502% | 10.2154% | 99.9246% |
| | If HE4 >1.8 then | | | | | | |
| | the following | | | | | | |
| | marker selected | | | | | | |
| | (best cutoff points | | | | | | |
| | used): CA125, | | | | | | |
| | MUC-1, MMP-7, | | | | | | |
| | Plau-R, GLY | | | | | | |
| 2 | Screen Test: | 90.2% | 62.2% | | | | |
| | If HE4 <=1.8 then | | | | | | |
| | test is negative | | | | | | |
| | Reflex Test: | 82% | 95.8% | 73.964% | 98.4124% | 10.4555% | 99.9337% |
| | If HE4 >1.8 then | | | | | | |
| | the following | | | | | | |
| | marker selected | | | | | | |
| | (Mean + 2 * std cutoff | | | | | | |
| | points used): | | | | | | |
| | CA125, MUC-1, | | | | | | |
| | MMP-7, Plau-R, | | | | | | |
| | Inhibin | | | | | | |
| 3† | Marker selected: | 80.8% | 98.5% | 80.8% | 98.5% | 11.8946% | 99.9512% |
| | PLAU-R, GLY, HE4, | | | | | | |
| | CA125, MMP-7, | | | | | | |
| | PAI-1 (Best cutoff | | | | | | |
| | points used) | | | | | | |
| 4† | Marker selected: | 75% | 99% | 75% | 99% | 15.8228% | 99.9368% |
| | PLAU-R, GLY, HE4, | | | | | | |
| | CA125, MMP-7 | | | | | | |
| | (Mean + 2 * std cutoff | | | | | | |
| | points used) | | | | | | |

*CA125 cutoff >= 35 in either best cutoff or mean + 2std cases
**Assume the population = 8,000,000; prevalence = 0.25%, the estimate cancer cases = 20,000 in the population.
† Although all listed biomarkers were assayed in a single step, additional algorithms were applied to obtain the values for sensitivity, specificity, PPV, and NPV indicated in the table.

Sensitivity, Specificity, PPV, and NPV Results Obtained with Application of “Test Rules”

In the current study, the biomarkers HE4, CA125, PLAU-R, glycodelin, Muc-1, PAI-1, MMP-7, and inhibin were the most frequently selected markers based on marker performance either as continuous or categorical variables. In contrast to the statistical methods described above, Table 53 provides the estimated sensitivity, specificity, PPV, and NPV (adjusted by disease prevalence) based on two test rules: (1) if any two of the above biomarkers are positive (i.e., overexpressed), then the test is declared positive or (2) if any 3 of the above biomarkers are positive (i.e., overexpressed), the test is declared positive. Contrary to the results presented above in Tables 51 and 52, though, the application of either test rule did not produce a high specificity rate, which in turn lead to an unacceptably low PPV (<1.5%) when adjusted for the low prevalence rate of ovarian cancer. The application of “test rules,” such as those described herein, is representative of the current state of the art in the identification of patients having an increased risk of ovarian cancer.

TABLE 53
Estimated Sensitivity, Specificity, PPV, and NPV Adjusted by Disease
Prevalence Based on Test Rules

| | | Performance* | | | |
|---|---|---|---|---|---|
| Example | Marker Selected and rule | Sensitivity | Specificity | PPV | NPV |

Overall Sample

| | | | | | |
|---|---|---|---|---|---|
| 1 | Any of two following | 97.475% | 51.654% | 0.5028% | 99.9878% |
| | markers are positive: HE4, | | | | |
| | CA125, Plau-R, GLY, | | | | |
| | MUC-1, PAI-1, MMP-7, | | | | |
| | Inhibin (best cutoff points | | | | |
| | used) | | | | |
| 2 | Any of 3 following | 91.414% | 78.372% | 1.0482% | 99.9726% |
| | markers are positive: HE4, | | | | |
| | CA125, Plau-R, GLY, | | | | |
| | MUC-1, PAI-1, MMP-7, | | | | |
| | Inhibin (best cutoff points | | | | |
| | used) | | | | |

Age > 55 Year Older

| | | | | | |
|---|---|---|---|---|---|
| 1 | Any of two following | 96.970% | 56.186% | 0.5516% | 99.9865% |
| | markers are positive: HE4, | | | | |
| | CA125, Plau-R, GLY, | | | | |
| | MUC-1, PAI-1, MMP-7, | | | | |
| | Inhibin (best cutoff points | | | | |
| | used) | | | | |
| 2 | Any of 3 following | 94.949% | 78.866% | 1.1135% | 99.9840% |
| | markers are positive: HE4, | | | | |
| | CA125, Plau-R, GLY, | | | | |
| | MUC-1, PAI-1, MMP-7, | | | | |
| | Inhibin (best cutoff points | | | | |
| | used) | | | | |

*CA125 cutoff>=35 in either best cutoff or mean + 2std cases
**Assume the population = 8,000,000; prevalence = 0.25%, estimated cancer cases = 20,000 in the population.

### Example 4

### Ovarian Biomarker Selection Study—Multiple Biomarker Analysis Using One-Step Screening Method

Expression of the biomarkers HE4, CA125, and glycodelin and combinations thereof for all patients and patient age over 55 was assessed using a one-step screening method, as described herein above. In particular, Table 54 provides the results for a one-step algorithm for patients over age 55. The test algorithm was based on a linear logistic regression model, and the actual value of each marker was used in the model. Linear logistic regression models (and other algorithms) are routine and well known in the art. See, for example, Fleiss (2003) Statistical Methods for Rates and Proportions (3^{rd} Ed.), which is herein incorporated by reference in its entirety. The algorithm offered 76.8% sensitivity at 96.4% specificity. A test outcome defined as positive was based on the following linear logistic regression model: ln(P/(1−P))=−4.2391+0.0888*CA125+0.1790*HE4+0.2349*GLY, where P is the conditional probability of ovarian cancer given CA125, HE4, and glycodelin being overexpressed in the patient body sample. A similar result was obtained with CA125 determined using manufacture's recommended cutoff value of ≧35 u/mL.

TABLE 54
Sensitivity and specificity of single-step screening assay to distinguish
ovarian cancer (all stages) from controls in patients over age 55

| Marker | Variable | Algorithm | Sensitivity | Specificity |
|---|---|---|---|---|
| HE4, CA125, | Actual value | 1 Step: based | 76.8% | 96.4% |
| GLYCODELIN | | on linear | | |
| | | logistic | | |
| | | model | | |
| HE4, CA125, | Actual value, | 1 Step: based | 71.4% | 95.4% |
| GLYCODELIN | except CA125 | on linear | | |
| | | logistic | | |
| | | model | | |

### Example 5

### Bootstrap Method of Analysis

### Validation of Study Methods

A bootstrap method of analysis was also conducted to assure a robust estimate of sensitivity and specificity for the above studies given that an independent and unique set of cancer sera was not available to validate the model. The bootstrap method consisted of artificially creating 1000 random datasets with replacement selection from the study sample set. A set of the most frequently selected markers in the study based on optimal performance was selected: HE4, CA125, PLAU-R, Glycodelin, MUC1 and PAI-1. A “test” was defined as follows: Test is positive if either (1) HE4 is positive and any one of CA125, Glycodelin, MMP-7, PLAU-R is positive or (2) HE4 is negative but CA125, Glycodelin, MMP-7, PLAU-R are all positive; otherwise, the “test” is negative. From the 1000 random samples, the mean/standard deviation and 95% confidence interval of the sensitivity/specificity of this defined test were obtained.

TABLE 55
Biomarker Sequence Information

| | Amino Acid Sequence | | Nucleotide Sequence | |
|---|---|---|---|---|
| | | Sequence | | Sequence |
| Biomarker Name | Accession No. | Identifier | Accession No. | Identifier |
| HE4 (Isoform 1) | NP_006094 | SEQ ID NO: 1 | NM_006103 | SEQ ID NO: 2 |
| HE4 (Isoform 3) | NP_542771 | SEQ ID NO: 3 | NM_080733 | SEQ ID NO: 4 |
| HE4 (Isoform 4) | NP_542772 | SEQ ID NO: 5 | NM_080734 | SEQ ID NO: 6 |
| HE4 (Isoform 2) | NP_542774 | SEQ ID NO: 7 | NM_080736 | SEQ ID NO: 8 |
| HE4 (Isoform 5) | NP_542773 | SEQ ID NO: 9 | NM_080735 | SEQ ID NO: 10 |
| KLK6 (Variant A) | NP_002765 | SEQ ID NO: 11 | NM_002774 | SEQ ID NO: 12 |
| KLK6 (Variant B) | NP_001012982 | SEQ ID NO: 13 | NM_001012964 | SEQ ID NO: 14 |
| KLK6 (Variant C) | NP_001012983 | SEQ ID NO: 15 | NM_001012965 | SEQ ID NO: 16 |
| KLK (Variant D) | NP_001012984 | SEQ ID NO: 17 | NM_001012966 | SEQ ID NO: 18 |
| KLK10 (Variant 1) | NP_002767 | SEQ ID NO: 19 | NM_002776 | SEQ ID NO: 20 |
| KLK10 (Variant 2) | NP_665895 | SEQ ID NO: 21 | NM_145888 | SEQ ID NO: 22 |
| Glycodelin (Variant | NP_001018059 | SEQ ID NO: 23 | NM_001018409 | SEQ ID NO: 24 |
| 1) | | | | |
| Glycodelin (Variant | NP_002562 | SEQ ID NO: 25 | NM_002571 | SEQ ID NO: 26 |
| 2) | | | | |
| PAI-1 | NP_000593 | SEQ ID NO: 27 | NM_000602 | SEQ ID NO: 28 |
| Muc-1 (Variant 1) | NP_002447 | SEQ ID NO: 29 | NM_002456 | SEQ ID NO: 30 |
| Muc-1 (Variant 2) | NP_001018016 | SEQ ID NO: 31 | NM_001018016 | SEQ ID NO: 32 |
| Muc-1 (Variant 3) | NP_001018017 | SEQ ID NO: 33 | NM_001018017 | SEQ ID NO: 34 |
| Muc-1 (Variant 4) | NP_001018021 | SEQ ID NO: 35 | NM_001018021 | SEQ ID NO: 36 |
| Alpha-1 anti-trypsin | NP_000286 | SEQ ID NO: 37 | NM_000295 | SEQ ID NO: 38 |
| Alpha-1 anti-trypsin | NP_001002235 | SEQ ID NO: 39 | NM_001002235 | SEQ ID NO: 40 |
| Alpha-1 anti-trypsin | NP_001002236 | SEQ ID NO: 41 | NM_001002236 | SEQ ID NO: 42 |
| PLAUR (Variant 1) | NP_002650 | SEQ ID NO: 43 | NM_002659 | SEQ ID NO: 44 |
| PLAUR (Variant 2) | NP_001005376 | SEQ ID NO: 45 | NM_001005376 | SEQ ID NO: 46 |
| PLAUR (Variant 3) | NP_001005377 | SEQ ID NO: 47 | NM_001005377 | SEQ ID NO: 48 |
| CTHRC1 | NP_612464 | SEQ ID NO: 49 | NM_138455 | SEQ ID NO: 50 |
| Inhibin (INHA) | NP_002182 | SEQ ID NO: 51 | NM_002191 | SEQ ID NO: 52 |
| Inhibin (INHBB) | NP_002184 | SEQ ID NO: 53 | NM_002193 | SEQ ID NO: 54 |
| Inhibin (INHBA) | NP_002183 | SEQ ID NO: 55 | NM_002192 | SEQ ID NO: 56 |
| CA125 (Muc-16) | NP_078966 | SEQ ID NO: 57 | NM_024690 | SEQ ID NO: 58 |
| MMP-7 | NP_002414 | SEQ ID NO: 59 | NM_002423 | SEQ ID NO: 60 |
| Prolactin | NP_000939 | SEQ ID NO: 61 | NM_000948 | SEQ ID NO: 62 |
| SLPI | NP_003055 | SEQ ID NO: 63 | NM_003064 | SEQ ID NO: 64 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. A single-step screening method for identifying patients with an increased likelihood of having ovarian cancer, the method comprising detecting the expression level of two biomarker proteins in a body sample, wherein the biomarker proteins are HE4 and CA125, and wherein overexpression of at least one of the biomarker proteins is indicative of the patient having an increased likelihood of having ovarian cancer.

2. The method of claim 1, wherein the body sample is a blood or serum sample.

3. The method of claim 1, wherein the expression level of each of the two biomarker proteins is compared to the expression level of each of the two biomarker proteins in a normal patient population.

4. The method of claim 1, wherein expression of the two biomarker proteins is detected using at least one antibody that specifically binds to HE4 and at least one antibody that specifically binds to CA125.

5. The method of claim 4, wherein expression of the two biomarker proteins is detected using an ELISA format or a multiplex bead-based immunoassay.

6. The method of claim 1, wherein the screening method is performed in an automated, semi-automated, or manual fashion.

7. The method of claim 1, wherein the method is used to detect early-stage ovarian cancer.

8. The method of claim 1, wherein the patients screened in accordance with the method of claim 1 are asymptomatic and have a family history of ovarian cancer or clinical risk factors indicating a high-risk for developing ovarian cancer.

9. The method of claim 1, wherein the screening method is performed in a population of post-menopausal female patients.

10. The method of claim 1, wherein patients identified as having an increased likelihood of having ovarian cancer are further subjected to additional diagnostic testing to determine if the patient has ovarian cancer, wherein the additional diagnostic testing is selected from the group consisting of pelvic examination, transvaginal ultrasound, CT scan, MRI, laparotomy, laparoscopy, and tissue sample biopsy.

11. The method of claim 10, wherein patients identified as having an increased likelihood of having ovarian cancer that are determined not to currently have ovarian cancer are further monitored on a regular basis for the development of ovarian cancer.

12. The method of claim 1, wherein the sensitivity of the screening method for identifying patients with an increased likelihood of having ovarian cancer is at least 75%.

13. The method of claim 1, wherein the specificity of the screening method for identifying patients with an increased likelihood of having ovarian cancer is at least 75%.
